(19) United States
(12) Patent Application Publication
(10) Pub. No.: US 2022/0411516 A1
(43) Pub. Date: Dec. 29, 2022

(54) DOSAGE REGIMEN FOR ANTI-EGFRVIII AGENTS

(71) Applicants: AMGEN INC., Thousand Oaks, CA (US); AMGEN RESEARCH (MUNICH) GMBH, Munich (DE)

(72) Inventors: Johannes Kast, South San Francisco, CA (US); Sabine Karin Stienen, Holzkirchen (DE); Vijay Upreti, San Francisco, CA (US); Marc Anthony Yago, San Francisco, CA (US)

(73) Assignees: AMGEN INC., Thousand Oaks, CA (US); AMGEN RESEARCH (MUNICH) GMBH, Munich (DE)

(21) Appl. No.: 17/775,543

(22) PCT Filed: Nov. 5, 2020
(86) PCT No.: PCT/US20/59169

§ 371 Date: May 9, 2022

Related U.S. Application Data
(60) Provisional application No. 62/931,975, filed on Nov. 7, 2019.

(51) Int. Cl. C07K 16/28; A61P 35/00

(52) U.S. Cl. CPC C07K 16/2863; A61P 35/00; C07K 16/2809; A61K 2039/545

(57) ABSTRACT

The invention provides a method for the treatment of EGFRvIII-positive cancer or glioblastoma, comprising administering to a subject in need thereof an initial dose of between about 15 μg/day to about 6000 μg/day of an anti-EGFRvIII agent. Diagnostic methods for assessing EGFRvIII expression are also provided.

### RELATED APPLICATIONS

This application claims the benefit under 35 U.S.C. § 119(e) to U.S. Provisional Application No. 62/931,975, filed Nov. 7, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Nov. 5, 2020, is named A-2518-WO-PCT_SL.txt and is 129,043 bytes in size.

### FIELD OF THE INVENTION

The present invention relates to dosage and administration of anti-EGFRvIII agents for the treatment of cancer.

### BACKGROUND OF THE INVENTION

Glioblastomas (GBM) belong to the group of highly malignant brain tumors representing one of the most lethal human cancers. The age-adjusted incidence of glioblastoma ranges from 0.59 to 3.69 per 100,000 persons worldwide. Despite aggressive surgical, radiologic and chemotherapeutic intervention, tumors progress within months or even weeks leading to an overall survival of 12 to 15 months with almost no change in prognosis since the FDA's approval of temozolomide (TMZ) in 2005 (Omuro & DeAngelis, JAMA, 2013; 310:1842-1850).

Upon recurrence after primary surgery, management of glioblastoma depends on age, performance status, histology, initial therapy response, time from original diagnosis, and whether the occurrence is local or diffuse. In patients with diffuse or multiple tumor recurrences, palliative care is a common choice. In patients with localized disease, combination of surgery, nitrosourea-based therapies, and radiation (standard re-irradiation or highly conformal radiation) is used, with poor results. A response to chemotherapy is unlikely after 2 consecutive agents have failed to produce a response (Stewart et al., Lancet 2002; 359(9311): 1011-1018). Moreover, no survival benefit has since been demonstrated for any new agent in a randomized clinical study (Mehta et al, Crit Rev Oncol Hematol. 2017; 111:60-65).

Epidermal growth factor receptor (EGFR) expression and enhanced EGF pathway signaling activity accompanied by amplification of the gene encoding EGFR have been documented in glioblastoma, almost exclusively in isocitrate dehydrogenase (IDH) wildtype glioblastoma (Louis et al., Acta Neuropathol. 2016; 131:803-820). About 50% of glioblastomas are positive for EGFR amplification, half of which express the accompanying EGFR mutation, encoding a truncated and constitutively active receptor termed EGFRvIII (Epidermal Growth Factor Receptor Variant III). Like native EGFR, mutant EGFRvIII is a membrane-bound receptor; however, the deletion results in a protein lacking 267 amino acid residues encompassing the extracellular ligand binding domain and characterized by a novel glycine residue occurring at the splice junction (Wong et al., Proc Natl Acad Sci USA. 1992; 89:2965-2969). While lacking an extracellular ligand binding domain, EGFRvIII has shown ligand-independent constitutive tyrosine kinase activity that stimulates downstream signaling pathways, which promote malignant growth (Mellinghoff et al., N Engl J Med. 2005; 353:2012-2024). According to one meta-analysis (Chen et al., Acta Neurol Scand. 2015; 132:310-322), there is currently insufficient evidence that either EGFR amplification or the EGFRvIII mutation has prognostic value in patients with glioblastoma. EGFRvIII is nevertheless considered a bona-fide tumor-specific antigen found exclusively on tumor cells thereby making it an attractive antitumor treatment strategy.

Accordingly, there is an urgent medical need for the development of therapies that target EGFRvIII.

### SUMMARY OF THE INVENTION

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

E1. A method for treating glioblastoma (GBM), comprising administering to a subject in need thereof an anti-EGFRvIII agent, at an initial dose of from about 15 μg/day to about 12000 μg/day.
E2. The method of E1, wherein said glioblastoma is EGFRvIII-positive glioblastoma.
E3. A method of treating EGFRvIII-positive cancer, comprising administering to a subject in need thereof an anti-EGFRvIII agent, at an initial dose of from about 15 μg/day to about 12000 μg/day.
E4. The method of E3, wherein said cancer is a solid tumor.
E5. The method of E3 or E4, wherein said cancer is a squamous cell tumor, such as non-small cell lung cancer (NSCLC).
E6. The method of E3 or E4, wherein said cancer is glioblastoma or malignant glioma.
E7. The method of any one of E1-E6, wherein said anti-EGFRvIII agent is a bispecific antibody construct comprising: a first binding domain that binds to human and macaque EGFRvIII, and a second binding domain that binds to human CD3.
E8. The method of E7, wherein said human EGFRvIII comprises the amino acid sequence of SEQ ID NO:1, and said macaque EGFRvIII comprises an amino acid sequence of SEQ ID NO:2.
E9. The method of E7 or E8, wherein said human CD3 comprises residues 1-27 of SEQ ID NO:123.
E10. The method of any one of E7-E9, wherein said human CD3 comprises the amino acid sequence of SEQ ID NO:123.
E11. The method of any one of E7-E10, wherein said EGFRvIII-binding domain comprises: (a) a heavy chain variable region (VH) that comprises: (i) a VH complementarity determining region one (CDR-H1) comprising the amino acid sequence of SEQ ID NO:3; (ii) a CDR-H2 comprising the amino acid sequence of SEQ ID NO:4; and (iii) a CDR-H3 comprising the amino acid sequence of SEQ ID NO:5; and (b) a light chain variable region (VL) that comprises: (i) a VL complementarity determining region one (CDR-11) comprising the amino acid sequence of SEQ ID NO:6; (ii) a CDR-L2 comprising the amino acid sequence of SEQ ID NO:7; and (iii) a CDR-L3 comprising the amino acid sequence of SEQ ID NO:8.
E12. The method of any one of E7-E11, wherein said EGFRvIII-binding domain comprises: a VH that comprises the amino acid sequence of SEQ ID NO:9, and a VL that comprises the amino acid sequence of SEQ ID NO:10.
E13. The method of E11 or E12, wherein said VH and VL are joined by a linker to form a single chain Fv (scFv).
E14. The method of E13, wherein said linker is a peptide linker comprising a sequence selected from any one of SEQ ID Nos. 114-122.
E15. The method of E13 or E14, wherein said linker comprises (Gly4Ser)x (SEQ ID NO: 143), where x is an integer of 1, 2, 3, or 4.
E16. The method of any one of E7-E15, wherein said EGFRvIII-binding domain comprises the amino acid sequence of SEQ ID NO:11.
E17. The method of any one of E7-E16, wherein said CD3-binding domain comprises:
(a) a VH that comprises: a CDR-H1 comprising the amino acid sequence of SEQ ID NO:18, a CDR-H2 comprising the amino acid sequence of SEQ ID NO:19, and a CDR-H3 comprising the amino acid sequence of SEQ ID NO:20; and a VL that comprises: a CDR-L1 comprising the amino acid sequence of SEQ ID NO:15, a CDR-L2 comprising the amino acid sequence of SEQ ID NO:16, and a CDR-L3 comprising the amino acid sequence of SEQ ID NO:17;
(b) a VH that comprises: a CDR-H1 comprising the amino acid sequence of SEQ ID NO:27, a CDR-H2 comprising the amino acid sequence of SEQ ID NO:28, and a CDR-H3 comprising the amino acid sequence of SEQ ID NO:29; and a VL that comprises: a CDR-L1 comprising the amino acid sequence of SEQ ID NO:24, a CDR-L2 comprising the amino acid sequence of SEQ ID NO:25, and a CDR-L3 comprising the amino acid sequence of SEQ ID NO:26;
(c) a VH that comprises: a CDR-H1 comprising the amino acid sequence of SEQ ID NO:36, a CDR-H2 comprising the amino acid sequence of SEQ ID NO:37, and a CDR-H3 comprising the amino acid sequence of SEQ ID NO:38; and a VL that comprises: a CDR-L1 comprising the amino acid sequence of SEQ ID NO:33, a CDR-L2 comprising the amino acid sequence of SEQ ID NO:34, and a CDR-L3 comprising the amino acid sequence of SEQ ID NO:35;
(d) a VH that comprises: a CDR-H1 comprising the amino acid sequence of SEQ ID NO:45, a CDR-H2 comprising the amino acid sequence of SEQ ID NO:46, and a CDR-H3 comprising the amino acid sequence of SEQ ID NO:47; and a VL that comprises: a CDR-L1 comprising the amino acid sequence of SEQ ID NO:42, a CDR-L2 comprising the amino acid sequence of SEQ ID NO:43, and a CDR-L3 comprising the amino acid sequence of SEQ ID NO:44;
(e) a VH that comprises: a CDR-H1 comprising the amino acid sequence of SEQ ID NO:54, a CDR-H2 comprising the amino acid sequence of SEQ ID NO:55, and a CDR-H3 comprising the amino acid sequence of SEQ ID NO:56; and a VL that comprises: a CDR-L1 comprising the amino acid sequence of SEQ ID NO:51, a CDR-L2 comprising the amino acid sequence of SEQ ID NO:52, and a CDR-L3 comprising the amino acid sequence of SEQ ID NO:53;
(f) a VH that comprises: a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; and a VL that comprises: a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62;
(g) a VH that comprises: a CDR-H1 comprising the amino acid sequence of SEQ ID NO:72, a CDR-H2 comprising the amino acid sequence of SEQ ID NO:73, and a CDR-H3 comprising the amino acid sequence of SEQ ID NO:74; and a VL that comprises: a CDR-L1 comprising the amino acid sequence of SEQ ID NO:69, a CDR-L2 comprising the amino acid sequence of SEQ ID NO:70, and a CDR-L3 comprising the amino acid sequence of SEQ ID NO:71;
(h) a VH that comprises: a CDR-H1 comprising the amino acid sequence of SEQ ID NO:81, a CDR-H2 comprising the amino acid sequence of SEQ ID NO:82, and a CDR-H3 comprising the amino acid sequence of SEQ ID NO:83; and a VL that comprises: a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80;
(i) a VH that comprises: a CDR-H1 comprising the amino acid sequence of SEQ ID NO:90, a CDR-H2 comprising the amino acid sequence of SEQ ID NO:91, and a CDR-H3 comprising the amino acid sequence of SEQ ID NO:92; and a VL that comprises: a CDR-L1 comprising the amino acid sequence of SEQ ID NO:87, a CDR-L2 comprising the amino acid sequence of SEQ ID NO:88, and a CDR-L3 comprising the amino acid sequence of SEQ ID NO:89;
(j) a VH that comprises: a CDR-H1 comprising the amino acid sequence of SEQ ID NO:99, a CDR-H2 comprising the amino acid sequence of SEQ ID NO:100, and a CDR-H3 comprising the amino acid sequence of SEQ ID NO:101; and a VL that comprises: a CDR-L1 comprising the amino acid sequence of SEQ ID NO:96, a CDR-L2 comprising the amino acid sequence of SEQ ID NO:97, and a CDR-L3 comprising the amino acid sequence of SEQ ID NO:98; OR
(k) a VH that comprises: a CDR-H1 comprising the amino acid sequence of SEQ ID NO:108, a CDR-H2 comprising the amino acid sequence of SEQ ID NO:109, and a CDR-H3 comprising the amino acid sequence of SEQ ID NO: 110; and a VL that comprises: a CDR-L1 comprising the amino acid sequence of SEQ ID NO:105, a CDR-L2 comprising the amino acid sequence of SEQ ID NO:106, and a CDR-L3 comprising the amino acid sequence of SEQ ID NO:107.
   E18. The method of any one of E7-E17, wherein said CD3-binding domain comprises:
(a) a VH that comprises the amino acid sequence of SEQ ID NO:21, and a VL that comprises the amino acid sequence of SEQ ID NO:22;
(b) a VH that comprises the amino acid sequence of SEQ ID NO:30, and a VL that comprises the amino acid sequence of SEQ ID NO:31;
(c) a VH that comprises the amino acid sequence of SEQ ID NO:39, and a VL that comprises the amino acid sequence of SEQ ID NO:40;
(d) a VH that comprises the amino acid sequence of SEQ ID NO:48, and a VL that comprises the amino acid sequence of SEQ ID NO:49;
(e) a VH that comprises the amino acid sequence of SEQ ID NO:57, and a VL that comprises the amino acid sequence of SEQ ID NO:58;
(f) a VH that comprises the amino acid sequence of SEQ ID NO:66, and a VL that comprises the amino acid sequence of SEQ ID NO:67;
(g) a VH that comprises the amino acid sequence of SEQ ID NO:75, and a VL that comprises the amino acid sequence of SEQ ID NO:76;
(h) a VH that comprises the amino acid sequence of SEQ ID NO:84, and a VL that comprises the amino acid sequence of SEQ ID NO:85;
(i) a VH that comprises the amino acid sequence of SEQ ID NO:93, and a VL that comprises the amino acid sequence of SEQ ID NO:94;
(j) a VH that comprises the amino acid sequence of SEQ ID NO:102, and a VL that comprises the amino acid sequence of SEQ ID NO:103; or
(k) a VH that comprises the amino acid sequence of SEQ ID NO:111, and a VL that comprises the amino acid sequence of SEQ ID NO:112.
   E19. The method of any one of E17 or E18, wherein said, the VH and VL of the CD3-binding domain are joined by a linker to form a single chain Fv (scFv).
   E20. The method of E19, wherein said linker is a peptide linker comprising a sequence selected from any one of SEQ ID Nos. 114-122.
   E21. The method of E19 or E20, wherein said linker comprises (Gly4Ser)x (SEQ ID NO: 143), where x is an integer of 1, 2, 3, or 4.
   E22. The method of any one of E7-E21, wherein said CD3-binding domain comprises the amino acid sequence of any one of SEQ ID NOs:23, 32, 41, 50, 59, 68, 77, 86, 95, 104, and 113.
   E23. The method of any one of E7-E22, wherein:
said EGFRvIII-binding domain comprises: (a) a heavy chain variable region (VH) that comprises: (i) a VH complementarity determining region one (CDR-H1) comprising the amino acid sequence of SEQ ID NO:3; (ii) a CDR-H2 comprising the amino acid sequence of SEQ ID NO:4; and (iii) a CDR-H3 comprising the amino acid sequence of SEQ ID NO:5; and (b) a light chain variable region (VL) that comprises: (i) a VL complementarity determining region one (CDR-11) comprising the amino acid sequence of SEQ ID NO:6; (ii) a CDR-L2 comprising the amino acid sequence of SEQ ID NO:7; and (iii) a CDR-L3 comprising the amino acid sequence of SEQ ID NO:8; and
said CD3-binding domain comprises: (a) a heavy chain variable region (VH) that comprises: (i) a VH complementarity determining region one (CDR-H1) comprising the amino acid sequence of SEQ ID NO:99; (ii) a CDR-H2 comprising the amino acid sequence of SEQ ID NO:100; and (iii) a CDR-H3 comprising the amino acid sequence of SEQ ID NO:101; and (b) a light chain variable region (VL) that comprises: (i) a VL complementarity determining region one (CDR-11) comprising the amino acid sequence of SEQ ID NO:96; (ii) a CDR-L2 comprising the amino acid sequence of SEQ ID NO:97; and (iii) a CDR-L3 comprising the amino acid sequence of SEQ ID NO:98.
   E24. The method of any one of E7-E23, wherein said EGFRvIII-binding domain comprises: a VH that comprises the amino acid sequence of SEQ ID NO:9, and a VL that comprises the amino acid sequence of SEQ ID NO:10; and wherein said CD3-binding domain comprises: a VH that comprises the amino acid sequence of SEQ ID NO:102, and a VL that comprises the amino acid sequence of SEQ ID NO:103.
   E25. The method of any one of E7-E24, wherein said EGFRvIII-binding domain comprises the amino acid sequence of SEQ ID NO:11, and said CD3-binding domain comprises the amino acid sequence of SEQ ID NO:104.
   E26. The method of any one of E7-E25, wherein said EGFRvIII-binding domain and said CD3-binding domain are joined by a linker.
   E27. The method of E26, wherein said linker is a peptide linker comprising a sequence selected from any one of SEQ ID Nos. 114-122.
   E28. The method of E27 or E28, wherein said linker comprises (Gly4Ser)x (SEQ ID NO: 143), where x is an integer of 1, 2, 3, or 4.
   E29. The method of any one E1-E28, wherein said anti-EGFRvIII agent comprises the amino acid sequence of SEQ ID NO: 12.
   E30. The method of any one E1-E29, wherein said anti-EGFRvIII agent comprises the amino acid sequence of SEQ ID NO: 13.
   E31. The method of any one E1-E30, wherein said anti-EGFRvIII agent is administered at an initial dose of: from about 15 μg/day to about 12000 μg/day, from about 15 μg/day to about 11000 μg/day, from about 15 μg/day to about 10000 μg/day, from about 15 μg/day to about 9000 μg/day, from about 15 μg/day to about 8000 μg/day, from about 15 μg/day to about 7000 μg/day, from about 45 μg/day to about 12000 μg/day, from about 45 μg/day to about 11000 μg/day, from about 45 μg/day to about 10000 μg/day, from about 45 μg/day to about 9000 μg/day, from about 45 μg/day to about 8000 μg/day, from about 45 μg/day to about 7000 μg/day, from about 150 μg/day to about 12000 μg/day, from about 150 μg/day to about 11000 μg/day, from about 150 μg/day to about 10000 μg/day, from about 150 μg/day to about 9000 μg/day, from about 150 μg/day to about 8000 μg/day, or from about 150 μg/day to about 7000 μg/day.
   E32. The method of any one E1-E31, wherein said anti-EGFRvIII agent is administered at an initial dose of from about 500 μg/day to about 6000 μg/day.
   E33. The method of any one E1-E32, wherein said anti-EGFRvIII agent is administered at an initial dose of from about 1000 μg/day to about 6000 μg/day.
   E34. The method of any one E1-E33, wherein said anti-EGFRvIII agent is administered at an initial dose of from about 1500 μg/day to about 6000 μg/day.
   E35. The method of any one E1-E34, wherein said anti-EGFRvIII agent is administered at an initial dose of from about 2000 μg/day to about 6000 μg/day.
   E36. The method of any one E1-E35, wherein said anti-EGFRvIII agent is administered at an initial dose of from about 3000 μg/day to about 6000 μg/day.
   E37. The method of any one E1-E36, wherein said anti-EGFRvIII agent is administered by intravenous (IV) infusion.
   E38. The method of any one E1-E37, wherein said anti-EGFRvIII agent is administered by continuous IV (cIV) infusion.
   E39. The method of any one E1-E38, wherein said anti-EGFRvIII agent is administered for at least 7 days at the initial dose of from about 15 μg/day to about 12000 μg/day.
   E40. The method of any one E1-E39, wherein said anti-EGFRvIII agent is administered for at least 7 days at the initial dose of from about 1500 μg/day to about 6000 μg/day.
   E41. The method of any one E1-E40, wherein said anti-EGFRvIII agent is administered for at least 7 days at the initial dose of from about 3000 μg/day to about 6000 μg/day.
   E42. The method of any one E1-E41, wherein said anti-EGFRvIII agent is administered for at least 14 days at the initial dose of from about 15 μg/day to about 12000 μg/day.
   E43. The method of any one E1-E42, wherein said anti-EGFRvIII agent is administered for at least 14 days at the initial dose of from about 1500 μg/day to about 6000 μg/day.
   E44. The method of any one E1-E43, wherein said anti-EGFRvIII agent is administered for at least 14 days at the initial dose of from about 3000 μg/day to about 6000 μg/day.
   E45. The method of any one E1-E44, wherein said anti-EGFRvIII agent is administered for at least 28 days at the initial dose of from about 15 μg/day to about 12000 μg/day.
   E46. The method of any one E1-E45, wherein said anti-EGFRvIII agent is administered for at least 28 days at the initial dose of from about 1500 μg/day to about 6000 μg/day.
   E47. The method of any one E1-E46, wherein said anti-EGFRvIII agent is administered for at least 28 days at the initial dose of from about 3000 μg/day to about 6000 μg/day.
   E48. The method of any one of E1-E47, further comprising administering to the subject one or more subsequent doses of the anti-EGFRvIII agent, at a dose of from about 15 μg/day to about 12000 μg/day.
   E49. The method of any one of E1-E48, further comprising administering to the subject one or more subsequent doses of the anti-EGFRvIII agent, at a dose of from about 1000 μg/day to about 6000 μg/day.
   E50. The method of any one of E1-E49, further comprising administering to the subject one or more subsequent doses of the anti-EGFRvIII agent, at a dose of from about 1500 μg/day to about 6000 μg/day.
   E51. The method of any one of E1-E50, further comprising administering to the subject one or more subsequent doses of the anti-EGFRvIII agent, at a dose of from about 3000 μg/day to about 6000 μg/day.
   E52. The method of any one of E1-E51, further comprising administering to said subject one or more subsequent doses of the anti-EGFRvIII agent, in an amount that is approximately the same or less than the initial dose.
   E53. The method of any one of E1-E52, wherein said one or more subsequent doses are dosed at least one week after the initial dose.
   E54. The method of any one of E1-E53, wherein said anti-EGFRvIII agent is administered at a 7-day on/7-day off cycle.
   E55. The method of any one of E1-E54, wherein said one or more subsequent doses are dosed at least two weeks after the initial dose.
   E56. The method of any one of E1-E53 and E55, wherein said anti-EGFRvIII agent is administered at a 14-day on/14-day off cycle.
   E57. The method of any one of E1-E53 and E55, wherein said anti-EGFRvIII agent is administered at a 28-day on/14-day off cycle.
   E58. The method of any one of E1-E57, further comprising administered an anti-inflammatory agent to said subject.
   E59. The method of E58, said anti-inflammatory agent is a corticosteroid.
   E60. The method of E59, wherein said corticosteroid is dexamethasone.
   E61. The method of any one of E58-E60, wherein said anti-inflammatory agent is administered prior to the treatment with anti-EGFRvIII agent.
   E62. The method of any one of E58-E60, wherein said anti-inflammatory agent is administered concurrently with the anti-EGFRvIII agent.
   E63. The method of any one of E1-E62, further comprising (a) obtaining a biological sample from said subject; and (b) detecting the presence of EGFRvIII or measuring the expression level of EGFRvIII in said sample.
   E64. The method of E63, wherein the presence of EGFRvIII or expression level of EGFRvIII is assessed by Immunohistochemistry (IHC), Fluorescence in situ hybridization (FISH), PCR, RT-PCR, or next-generation sequencing (NGS).
   E65. The method of E63 or E64, wherein the presence of EGFRvIII or expression level of EGFRvIII is assessed by Immunohistochemistry (IHC).
   E66. The method of E65, wherein the presence of EGFRvIII or expression level of EGFRvIII is assessed by H-score.
   E67 The method of E66, wherein said subject has an H-score of from about 8 to about 280.
   E67a. The method of E66, wherein said subject has an H-score of from about 8 to about 300.
   E68. The method of any one of E63-E67 and E67a, wherein the presence of EGFRvIII or expression level of EGFRvIII is assessed by an antibody, or antigen binding fragment thereof, that binds to EGFRvIII.
   E69. The method of E68, wherein said antibody, or antigen-binding fragment thereof, comprises:
(a) a heavy chain variable region (VH) that comprises: (i) a VH complementarity determining region one (CDR-H1) comprising the amino acid sequence of SEQ ID NO:127; (ii) a CDR-H2 comprising the amino acid sequence of SEQ ID NO:128; and (iii) a CDR-H3 comprising the amino acid sequence of SEQ ID NO:129; and (b) a light chain variable region (VL) that comprises: (i) a VL complementarity determining region one (CDR-L1) comprising the amino acid sequence of SEQ ID NO:130; (ii) a CDR-L2 comprising the amino acid sequence of SEQ ID NO:131; and (iii) a CDR-L3 comprising the amino acid sequence of SEQ ID NO:132.
   E70. The method of E68 or E69, wherein said antibody, or antigen-binding fragment thereof, comprises: a VH that comprises an amino acid sequence that is at least 90%, at least 91%, at least 92%, at least 93%, at least 94%, at least 95%, at least 96%, at least 97%, at least 98%, at least 99%, or 100% identical to SEQ ID NO:133, and a VL that comprises an amino acid sequence that is at least 90%, at least 91%, at least 92%, at least 93%, at least 94%, at least 95%, at least 96%, at least 97%, at least 98%, at least 99%, or 100% identical to SEQ ID NO:134.
   E71. The method of any one of E68-E70, wherein said antibody, or antigen-binding fragment thereof, comprises: a VH that comprises an amino acid sequence that is at least 90% identical to SEQ ID NO:133, and a VL that comprises an amino acid sequence that is at least 90% identical to SEQ ID NO:134.
   E72. The method of any one of E68-E71, wherein said antibody, or antigen-binding fragment thereof, comprises: a VH that comprises an amino acid sequence that is at least 95% identical to SEQ ID NO:133, and a VL that comprises an amino acid sequence that is at least 95% identical to SEQ ID NO:134.
   E73. The method of any one of E68-E72, wherein said antibody, or antigen-binding fragment thereof, comprises: a VH that comprises the amino acid sequence of SEQ ID NO:133, and a VL that comprises the amino acid sequence of SEQ ID NO:134.
   E74. The method of any one of E68-E73, wherein said antibody, or antigen-binding fragment thereof, comprises a heavy constant region (CH).
   E75. The method of E75, wherein said CH comprises a murine CH.
   E76. The method of E74 or E75, wherein said CH comprises a murine IgG1 isotype heavy chain constant region.
   E77. The method of any one of E74-E76, wherein said CH comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:135.
   E78. The method of any one of E68-E77, wherein said antibody, or antigen-binding fragment thereof, comprises a light chain constant region (CL).
   E79. The method of E78, wherein said CL comprises a murine CL.
   E80. The method of E78 or E79, wherein said CL comprises a murine kappa isotype light chain constant region.
   E81. The method of any one of E78-E80, wherein said CL comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:136.
   E82. The method of any one of E68-E81, wherein said antibody, or antigen-binding fragment thereof, comprises: a heavy chain that comprises an amino acid sequence that is at least 90%, at least 91%, at least 92%, at least 93%, at least 94%, at least 95%, at least 96%, at least 97%, at least 98%, at least 99%, or 100% identical to SEQ ID NO:137, and a light chain that comprises an amino acid sequence that is at least 90%, at least 91%, at least 92%, at least 93%, at least 94%, at least 95%, at least 96%, at least 97%, at least 98%, at least 99%, or 100% identical to SEQ ID NO:138.
   E83. The method of any one of E68-E82, wherein said antibody, or antigen-binding fragment thereof, comprises: a heavy chain that comprises an amino acid sequence that is at least 90% identical to SEQ ID NO:137, and a light chain that comprises an amino acid sequence that is at least 90% identical to SEQ ID NO:138.
   E84. The method of any one of E68-E83, wherein said antibody, or antigen-binding fragment thereof, comprises: a heavy chain that comprises an amino acid sequence that is at least 95% identical to SEQ ID NO:137, and a light chain that comprises an amino acid sequence that is at least 95% identical to SEQ ID NO:138.
   E85. The method of any one of E68-E84, wherein said antibody, or antigen-binding fragment thereof, comprises: a heavy chain that comprises the amino acid sequence of SEQ ID NO:137, and a light chain that comprises the amino acid sequence of SEQ ID NO:138.
   E86. The method of any one of E1-E2, and E6-E85, wherein said glioblastoma is newly diagnosed glioblastoma (nGBM).
   E87. The method of any one of E1-E2, and E6-E85, wherein said glioblastoma is recurrent glioblastoma (rGBM).
   E88. The method of any one of E1-E87, wherein said subject is a human.
   E89. An assay kit for the detection of EGFRvIII in a mammalian tissue or cell sample, comprising:
a first monoclonal antibody that binds to EGFRvIII, and comprises: (a) a heavy chain variable region (VH) that comprises: (i) a VH complementarity determining region one (CDR-H1) comprising the amino acid sequence of SEQ ID NO:127; (ii) a CDR-H2 comprising the amino acid sequence of SEQ ID NO:128; and (iii) a CDR-H3 comprising the amino acid sequence of SEQ ID NO:129; and (b) a light chain variable region (VL) that comprises: (i) a VL complementarity determining region one (CDR-L1) comprising the amino acid sequence of SEQ ID NO:130; (ii) a CDR-L2 comprising the amino acid sequence of SEQ ID NO:131; and (iii) a CDR-L3 comprising the amino acid sequence of SEQ ID NO:132; and a second antibody that binds to said first antibody.
   E90. The assay kit of E89, wherein said first antibody comprises: a VH that comprises an amino acid sequence that is at least 90%, at least 91%, at least 92%, at least 93%, at least 94%, at least 95%, at least 96%, at least 97%, at least 98%, at least 99%, or 100% identical to SEQ ID NO:133, and a VL that comprises an amino acid sequence that is at least 90%, at least 91%, at least 92%, at least 93%, at least 94%, at least 95%, at least 96%, at least 97%, at least 98%, at least 99%, or 100% identical to SEQ ID NO:134.
   E91. The assay kit of E89 or E90, wherein said first antibody comprises: a VH that comprises an amino acid sequence that is at least 90% identical to SEQ ID NO:133, and a VL that comprises an amino acid sequence that is at least 90% identical to SEQ ID NO:134.
   E92. The assay kit of any one of E88-E91, wherein said first antibody comprises: a VH that comprises an amino acid sequence that is at least 95% identical to SEQ ID NO:133, and a VL that comprises an amino acid sequence that is at least 95% identical to SEQ ID NO:134.
   E93. The assay kit of any one of E88-E92, wherein said first antibody comprises: a VH that comprises the amino acid sequence of SEQ ID NO:133, and a VL that comprises the amino acid sequence of SEQ ID NO:134.
   E94. The assay kit of any one of E88-E93, wherein said first antibody comprises a murine heavy constant region (CH).
   E95. The assay kit of any one of E88-E94, wherein said first antibody comprises a murine IgG1 isotype heavy chain constant region.
   E96. The assay kit of any one of E88-E95, wherein said first antibody comprises a CH sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:135.
   E97. The assay kit of any one of E88-E96, wherein said first antibody comprises a murine light chain constant region (CL).
   E98. The assay kit of any one of E88-E97, wherein said first antibody comprises a murine kappa isotype light chain constant region.
   E99. The assay kit of any one of E88-E98, wherein said first antibody comprises a CL sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:136.
   E100. The assay kit of any one of E88-E99, wherein said first antibody comprises: a heavy chain that comprises an amino acid sequence that is at least 90%, at least 91%, at least 92%, at least 93%, at least 94%, at least 95%, at least 96%, at least 97%, at least 98%, at least 99%, or 100% identical to SEQ ID NO:137, and a light chain that comprises an amino acid sequence that is at least 90%, at least 91%, at least 92%, at least 93%, at least 94%, at least 95%, at least 96%, at least 97%, at least 98%, at least 99%, or 100% identical to SEQ ID NO:138.
   E101. The assay kit of any one of E88-E100, wherein said first antibody comprises: a heavy chain that comprises an amino acid sequence that is at least 90% identical to SEQ ID NO:137, and a light chain that comprises an amino acid sequence that is at least 90% identical to SEQ ID NO:138.
   E102. The assay kit of any one of E88-E101, wherein said first antibody comprises: a heavy chain that comprises an amino acid sequence that is at least 95% identical to SEQ ID NO:137, and a light chain that comprises an amino acid sequence that is at least 95% identical to SEQ ID NO:138.
   E103. The assay kit of any one of E88-E102, wherein said first antibody comprises: a heavy chain that comprises the amino acid sequence of SEQ ID NO:137, and a light chain that comprises the amino acid sequence of SEQ ID NO:138.
   E104. The assay kit of any one of E88-E103, wherein said second antibody binds to a murine immunoglobulin constant region.
   E105. The assay kit of any one of E88-E104, wherein a detectable label is attached to said second antibody.
   E106. The assay kit of any one of E88-E105, wherein said detectable label is a radioactive agent, a chemiluminescent agent, a fluorescent agent, or a phosphorescent agent.
   E107. The assay kit of any one of E88-E106, further comprising instructions to use said kit.
   E108. A method for assessing the presence of EGFRvIII, or the expression level of EGFRvIII in a subject, comprising: (i) incubating a tissue sample from said subject with a first antibody that binds to EGFRvIII, wherein said first antibody comprises a VH sequence comprising SEQ ID NO: 133 and a VL sequence comprising SEQ ID NO:134; and wherein the concentration of the first antibody is about 3 μg/ml or lower; (ii) removing the excess, unbound first antibody; and (iii) incubating the tissue sample with a second antibody, wherein the second antibody binds to a constant domain of said first antibody.
   E109. The method of E108, wherein the concentration of said first antibody is about 2.75 μg/ml during the incubation step.
   E110. The method of E108, wherein the concentration of said first antibody is about 1.375 μg/ml during the incubation step.
   E111. The method of any one of E108-110, wherein said first antibody comprises a heavy chain sequence comprising SEQ ID NO:137, and a light chain sequence comprising SEQ ID NO:138.
   E112. An anti-EGFRvIII agent for use in a method as set forth in any one of embodiments E1-E88.
   E113. Use of an anti-EGFRvIII agent as set forth in E112 in the preparation of a medicament for the treatment of glioblastoma.
   E114. Use of an anti-EGFRvIII agent as set forth in E112 in the preparation of a medicament for the treatment of an EGFRvIII-positive cancer.

### BRIEF DESCRIPTION OF THE FIGURES

FIG. 1 is a schematic illustration of dose levels and treatment scheme for the Phase 1 study. *1: 1 subject received accidental 1500 ug/day instead of 15 ug/day for 1 week in cycle 1. rGBM: relapsed glioblastoma; 1stLGBM: maintenance treatment for GBM after primary surgery, adjunct radiochemotherapy+/−maintenance temozolomide according to local standards.

FIG. 2 shows the predicted AMG 596 serum/CSF concentrations and minimally efficacious human exposures. Day 0-day 28 curves represents cohorts 1-10, from bottom to top respectively. The dash lines across the graphs represent, from bottom to top, EC20, EC50, and EC90 values that were calculated based on in vitro experiments. The EC20 is 0.07 ng/ml, EC50 is 0.41 ng/mL, and EC90 is 1.8 ng/mL.

FIG. 3A shows the mean exposures during 1st cycle for 28-day on/14-day off Civ cohorts 2-6 & expanded access protocol subject (1500 μg/day initially then 15 μg/day). FIG. 3B shows the mean exposures during 1st cycle for 28-day on/14-day off Civ cohorts 2-8 & expanded access protocol subject. Efficacious Exposure Prediction was based on observed 3.6% CSF penetration in Cynomolgus monkeys.

FIG. 4A is a waterfall plot showing changes in tumor size in patients. FIG. 4B is a spider plot showing changes in tumor size in patients overtime. FIG. 4C summaries the evaluations by External Read for subject 42001013. Corresponding MRI images for Baseline (Aug. 30, 2018), Follow-up 1 (Dec. 4, 2018), Follow-up 2 (Feb. 26, 2019) and Follow-up 3 (Apr. 17, 2019) also confirms tumor shrinkage (images not shown). *Patient 13266001006 received an overdose (1500 μg/day) and has entered expanded access study based on FDA recommendation to receive 1500 μg/day during week 1, followed by 15 μg/day during weeks 2-4, followed by a 2-week break; ^{†}Patient13242001041 had a PR per target lesions, but overall had PD at the initial scan due to clinical deterioration. The patient discontinued treatment due to an adverse event. NA=not available; PD=progressive disease; PR=partial response; SD=stable disease. FIG. 4D is an updated waterfall plot showing changes in tumor size in patients —Group 1 (Safety Analysis Set). Safety Analysis Set includes all subjects who are enrolled and received at least 1 dose of AMG 596. Minimum change from baseline is plotted per subject. Reporting period: Inception to 13 Aug. 2020.

FIG. 5 is a lesion overview chart from the tumor evaluation by External Reader for subject 66001006. The central read supports unconfirmed PR. MRI images (not shown) also confirms tumor shrinkage, from 528.3 mm² baseline (Aug. 29, 2018) to 221.6 mm² during Follow-up 1(Oct. 15, 2018), about 58.1% reduction.

FIG. 6A is a plot showing that Semi-quantitative EGFRvIII expression analysis revealed a median H-score of 115 (range, 8-280). PD=progressive disease; PR=partial response; SD=stable disease. *Unknown includes patients for whom assessment of response has not yet been performed. FIG. 6B is an updated plot (29 patients) showing that Semi-quantitative EGFRvIII expression analysis revealed a median H-score of 127 (range, 1-280).

FIG. 7A is a table summarizing Baseline Characteristics of the enrolled subjects as of cut-off date 13 Aug. 2020. ECOG performance status^{a}: Eastern Cooperative Oncology Group performance status. FIG. 7B is a table summarizing Subject Disposition as of cut-off date 13 Aug. 2020.

FIG. 8 is a table summarizing Subject Incidence of Treatment-emergent Neurological Adverse Events—Group 1 (Safety Analysis Set). Safety Analysis Set includes all subjects who were enrolled and received at least 1 dose of AMG 596. Group 1 includes subjects with recurrent disease confirmed by MRI. Subject 13266001006 was enrolled to Cohort 2b (15 mcg/day cIV 28 days on followed by 14 days off). This subject discontinued the study 20160132 as the subject received an overdose (1500 mcg/day) and subsequently entered expanded access study 20180427 based on FDA recommendation to receive 1,500 mcg/day during week 1, followed by 15 mcg/day during weeks 2-4, followed by a 2-week break. The analysis was coded using MedDRA version 23.0. Severity of each adverse event was graded using CTCAE version 4.0 criteria, for Cytokine Release Syndrome (CRS) events, revised grading system is used per protocol (Lee et al, 2014). Reporting Period: Inception to 13 Aug. 2020.

FIG. 9 is a table summarizing Best Overall Response—Group 1 (Safety Analysis Set). Safety Analysis Set includes all subjects who are enrolled and received at least 1 dose of AMG 596. Group 1 includes subjects with recurrent disease confirmed by MRI. Best overall response is the most favorable post baseline response across all the assessments. Subjects discontinued treatment due to disease progression without a follow-up scan are considered progressors. Subject 13266001006 was enrolled to Cohort 2b (15 mcg/day cIV 28 days on followed by 14 days off). This subject has discontinued the study 20160132 as the subject received an overdose (1500 mcg/day) and had entered expanded access study 20180427 based on FDA recommendation to receive 1,500 mcg/day during week 1, followed by 15 mcg/day during weeks 2-4, followed by a 2-week break. [1]: Inevaluable indicates post baseline scans were not readable. [2]: Not done indicates that either no post baseline scans were performed or no post baseline scans are available. Reporting Period: Inception to 13 Aug. 2020.

FIG. 10 shows the result of MRI scans and tumor measurements (external reader) for the patient with ongoing confirmed PR. Blue arrows indicate time points of dose escalation.

### DETAILED DESCRIPTION OF THE INVENTION

### 1. Overview

As disclosed and exemplified herein, a Phase 1 clinical study for treatment of glioblastoma was conducted, using a bispecific protein (AMG 596) that targets EGFRvIII and CD3.

Under normal circumstances, blood-brain barrier (BBB) excludes the vast majority of therapeutic molecules from penetrating the brain, and AMG 596 would be considered too big to pass through BBB. Surprisingly, it was discovered that AMG 596 is penetrating the BBB and can effectively bind to EGFRvIII-expressing intracranial tumors. Limited preliminary AMG 596 exposure data suggests that serum to CSF penetration is variable between subjects and approximately between 0.3% and 1.7%. Pharmacodynamic activity can be seen in almost all subjects with EGFRvIII positive recurrent glioblastoma and with steady state exposure above 2-5 ng/mL or AMG 596 doses as low as 15 mcg per day (see examples 5, 6, and 7). Unexpectedly, at least in some subjects, the observed efficacy and pharmacodynamic activity seem to suggest that AMG 596 penetrates BBB better than what was predicted based on computer modeling. Therefore, predicted efficacious dose for AMG 596 can be as low as 15 μg per day administered as continuous intravenous infusion for 28 days per treatment cycle to subjects with recurrent glioblastoma assumed to be positive EGFRvIII-positive.

In some circumstances, especially for patients with recurrent EGFRvIII-positive glioblastoma, it would be desirable to achieve objective antitumor responses as early as after a first treatment cycle. Based on pharmacodynamic activity, a serum exposure of at least 79 ng/mL is desirable. This correlates to a dose range of from about 1500 μg per day to about 6000 μg per day in subjects with recurrent EGFRvIII-positive glioblastoma. If prophylactic dexamethasone is given prior to start of the AMG 596 infusion, a higher AMG 596 dose may be required due to potential impact of dexamethasone on T cell proliferation. Therefore, with prophylactic dexamethasone treatment, the preferred AMG 596 dose range would be from about 3000 μg per day to about 6000 μg per day in subjects with recurrent EGFRvIII-positive glioblastoma. In addition, a dose up to 12000 μg per day can be taken into consideration.

Treatment duration in patients with recurrent EGFRvIII-positive glioblastoma can be several months until up to 2 years or longer. A long treatment duration can result in slow but continuous tumor shrinkage. If a start dose below 1500 μg per day is administered, a dose escalation to a dose of 1500 μg per day or 3000 μg per day or even higher may trigger additional tumor shrinkage. The break between treatment cycles preferably is about 2 weeks. However, with longer treatment duration breaks of 3 or 4 weeks can be acceptable, in particular, if a next cycle will start at a higher dose.

### 2. Definitions

Some of exemplary bispecific anti-EGFRvIII agents disclosed herein (such as BiTE® molecules) are recombinant protein constructs comprising two binding domains, each domain derived from an antigen-binding fragment of a full-length antibody. Such antigen-binding fragment retains the ability to specifically bind to an antigen (preferably with substantially the same binding affinity). Examples of an antigen-binding fragment includes (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, and (v) a dAb fragment (Ward et al., 1989 Nature 341:544-546), which consists of a VH domain. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423-426 (1988) and Huston et al., 1988. Proc. Natl. Acad. Sci. USA 85:5879-5883.

A “variable domain” refers to the variable region of the antibody light chain (VL) or the variable region of the antibody heavy chain (VH), either alone or in combination. As known in the art, the variable regions of the heavy and light chains each consist of four framework regions (FR) connected by three complementarity determining regions (CDRs), and contribute to the formation of the antigen-binding site of antibodies.

The “Complementarity Determining Regions” (CDRs) of exemplary EGFRvIII-binding domains and CD3-binding domains are provided in the Sequence Table. The CDRs can be defined according to Kabat, Chothia, the accumulation of both Kabat and Chothia. AbM, contact, North, and/or conformational definitions or any method of CDR determination well known in the art. See, e.g., Kabat et al., 1991, Sequences of Proteins of Immunological Interest, 5th ed. (hypervariable regions); Chothia et al., 1989, Nature 342:877-883 (structural loop structures). AbM definition of CDRs is a compromise between Kabat and Chothia and uses Oxford Molecular's AbM antibody modeling software (Accelrys®). The identity of the amino acid residues in a particular antibody that make up a CDR can be determined using methods well known in the art. [26] The term “treatment” includes prophylactic and/or therapeutic treatments. If it is administered prior to clinical manifestation of a condition, the treatment is considered prophylactic. Therapeutic treatment includes. e.g., ameliorating or reducing the severity of a disease, or shortening the length of the disease.

“About” or “approximately,” when used in connection with a measurable numerical variable, refers to the indicated value of the variable and to all values of the variable that are within the experimental error of the indicated value (e.g. within the 95% confidence interval for the mean) or ±10% of the indicated value, whichever is greater. Numeric ranges are inclusive of the numbers defining the range.

### 3. Anti-EGFRvIII Agents

The epidermal growth factor receptor (EGFR) is a pivotal regulator of normal cellular growth in tissues of epithelial origin. Dysregulated EGFR signaling (resulting from mechanisms such as cell-surface overexpression, autocrine activation and EGFR gene mutation) contributes to the formation of many epithelial malignancies in humans. Several EGFR mutations have been described. The most common extracellular mutation is EGFRvIII (also known as de2-7EGFR and ΔEGFR). EGFRvIII is a tumor-specific mutation that results from in-frame deletion of 801 base pairs spanning exons 2-7 of the coding sequence. This deletion removes 267 amino acids from the extracellular domain, creating a junction site between exons 1 and 8 and a new glycine residue. EGFRvIII has a molecular mass of approximately 145 kDa. The amino acid sequences of human and cynomolgus EGFRvIII are shown as SEQ ID Nos. 1 and 2, respectively.

An exemplary anti-EGFRvIII agent is a bispecific molecule that binds EGFRvIII and CD3, such as a BiTE® (bispecific T cell engager) molecule. BiTE® molecules are recombinant protein constructs made from two flexibly linked binding domains, each domain derived from antibodies. One binding domain of BiTE® molecule is specific for a tumor-associated surface antigen (such as EGFRvIII); the second binding domain is specific for CD3, a subunit of the T cell receptor complex on T cells. By their design, BiTE® molecules are uniquely suited to transiently connect T cells with target cells and, at the same time, potently activate the inherent cytolytic potential of T cells against target cells. See e.g., WO 99/54440, WO 2005/040220, and WO 2008/119567.

Accordingly, in some embodiments, the anti-EGFRvIII agent described comprises two binding domains: the first domain binds EGFRvIII (preferably human EGFRvIII), and the second domain binds CD3 (preferably human CD3). Exemplary CD3 sequences are provided as SEQ ID Nos. 123-126. Preferably, the second domain binds to residues 1-27 of SEQ ID NO:123. Alternatively, the second domain may bind to residues 1-27 of any one of SEQ ID NOs:124-126.

In certain embodiments, the EGFRvIII-binding domain comprises: (a) a heavy chain variable region (VH) that comprises: (i) a VH complementarity determining region one (CDR-H1) comprising the amino acid sequence of SEQ ID NO:3; (ii) a CDR-H2 comprising the amino acid sequence of SEQ ID NO:4; and (iii) a CDR-H3 comprising the amino acid sequence of SEQ ID NO:5; and (b) a light chain variable region (VL) that comprises: (i) a VL complementarity determining region one (CDR-L1) comprising the amino acid sequence of SEQ ID NO:6; (ii) a CDR-L2 comprising the amino acid sequence of SEQ ID NO:7; and (iii) a CDR-L3 comprising the amino acid sequence of SEQ ID NO:8.

In certain embodiments, the EGFRvIII-binding domain comprises: a VH that comprises the amino acid sequence of SEQ ID NO:9, and a VL that comprises the amino acid sequence of SEQ ID NO:10. In some embodiments, the VH and VL are joined by a linker to form a single chain Fv (scFv). In some embodiments, the linker is a peptide linker comprising a sequence selected from any one of SEQ ID Nos. 114-122. In some embodiments, the linker is a GS liker, such as Gly-Gly-Gly-Gly-Ser (G4S, SEQ ID NO: 115), or polymers thereof, i.e. (Gly4Ser)x (SEQ ID NO: 144), where x is an integer of 1 or greater (e.g. 2 or 3) (e.g., SEQ ID Nos. 121, 122).

In certain embodiments, the EGFRvIII-binding domain comprises the amino acid sequence of SEQ ID NO:11.

In certain embodiments, the CD3-binding domain comprises:
(a) a VH that comprises: a CDR-H1 comprising the amino acid sequence of SEQ ID NO:18, a CDR-H2 comprising the amino acid sequence of SEQ ID NO:19, and a CDR-H3 comprising the amino acid sequence of SEQ ID NO:20; and a VL that comprises: a CDR-L1 comprising the amino acid sequence of SEQ ID NO:15, a CDR-L2 comprising the amino acid sequence of SEQ ID NO:16, and a CDR-L3 comprising the amino acid sequence of SEQ ID NO:17;
(b) a VH that comprises: a CDR-H1 comprising the amino acid sequence of SEQ ID NO:27, a CDR-H2 comprising the amino acid sequence of SEQ ID NO:28, and a CDR-H3 comprising the amino acid sequence of SEQ ID NO:29; and a VL that comprises: a CDR-L1 comprising the amino acid sequence of SEQ ID NO:24, a CDR-L2 comprising the amino acid sequence of SEQ ID NO:25, and a CDR-L3 comprising the amino acid sequence of SEQ ID NO:26;
(c) a VH that comprises: a CDR-H1 comprising the amino acid sequence of SEQ ID NO:36, a CDR-H2 comprising the amino acid sequence of SEQ ID NO:37, and a CDR-H3 comprising the amino acid sequence of SEQ ID NO:38; and a VL that comprises: a CDR-L1 comprising the amino acid sequence of SEQ ID NO:33, a CDR-L2 comprising the amino acid sequence of SEQ ID NO:34, and a CDR-L3 comprising the amino acid sequence of SEQ ID NO:35;
(d) a VH that comprises: a CDR-H1 comprising the amino acid sequence of SEQ ID NO:45, a CDR-H2 comprising the amino acid sequence of SEQ ID NO:46, and a CDR-H3 comprising the amino acid sequence of SEQ ID NO:47; and a VL that comprises: a CDR-L1 comprising the amino acid sequence of SEQ ID NO:42, a CDR-L2 comprising the amino acid sequence of SEQ ID NO:43, and a CDR-L3 comprising the amino acid sequence of SEQ ID NO:44;
(e) a VH that comprises: a CDR-H1 comprising the amino acid sequence of SEQ ID NO:54, a CDR-H2 comprising the amino acid sequence of SEQ ID NO:55, and a CDR-H3 comprising the amino acid sequence of SEQ ID NO:56; and a VL that comprises: a CDR-L1 comprising the amino acid sequence of SEQ ID NO:51, a CDR-L2 comprising the amino acid sequence of SEQ ID NO:52, and a CDR-L3 comprising the amino acid sequence of SEQ ID NO:53;
(f) a VH that comprises: a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; and a VL that comprises: a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62;
(g) a VH that comprises: a CDR-H1 comprising the amino acid sequence of SEQ ID NO:72, a CDR-H2 comprising the amino acid sequence of SEQ ID NO:73, and a CDR-H3 comprising the amino acid sequence of SEQ ID NO:74; and a VL that comprises: a CDR-L1 comprising the amino acid sequence of SEQ ID NO:69, a CDR-L2 comprising the amino acid sequence of SEQ ID NO:70, and a CDR-L3 comprising the amino acid sequence of SEQ ID NO:71;
(h) a VH that comprises: a CDR-H1 comprising the amino acid sequence of SEQ ID NO:81, a CDR-H2 comprising the amino acid sequence of SEQ ID NO:82, and a CDR-H3 comprising the amino acid sequence of SEQ ID NO:83; and a VL that comprises: a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80;
(i) a VH that comprises: a CDR-H1 comprising the amino acid sequence of SEQ ID NO:90, a CDR-H2 comprising the amino acid sequence of SEQ ID NO:91, and a CDR-H3 comprising the amino acid sequence of SEQ ID NO:92; and a VL that comprises: a CDR-L1 comprising the amino acid sequence of SEQ ID NO:87, a CDR-L2 comprising the amino acid sequence of SEQ ID NO:88, and a CDR-L3 comprising the amino acid sequence of SEQ ID NO:89;
(j) a VH that comprises: a CDR-H1 comprising the amino acid sequence of SEQ ID NO:99, a CDR-H2 comprising the amino acid sequence of SEQ ID NO:100, and a CDR-H3 comprising the amino acid sequence of SEQ ID NO:101; and a VL that comprises: a CDR-L1 comprising the amino acid sequence of SEQ ID NO:96, a CDR-L2 comprising the amino acid sequence of SEQ ID NO:97, and a CDR-L3 comprising the amino acid sequence of SEQ ID NO:98; OR
(k) a VH that comprises: a CDR-H1 comprising the amino acid sequence of SEQ ID NO:108, a CDR-H2 comprising the amino acid sequence of SEQ ID NO:109, and a CDR-H3 comprising the amino acid sequence of SEQ ID NO: 110; and a VL that comprises: a CDR-L1 comprising the amino acid sequence of SEQ ID NO:105, a CDR-L2 comprising the amino acid sequence of SEQ ID NO:106, and a CDR-L3 comprising the amino acid sequence of SEQ ID NO:107.

In certain embodiments, the CD3-binding domain comprises:
(a) a VH that comprises the amino acid sequence of SEQ ID NO:21, and a VL that comprises the amino acid sequence of SEQ ID NO:22;
(b) a VH that comprises the amino acid sequence of SEQ ID NO:30, and a VL that comprises the amino acid sequence of SEQ ID NO:31;
(c) a VH that comprises the amino acid sequence of SEQ ID NO:39, and a VL that comprises the amino acid sequence of SEQ ID NO:40;
(d) a VH that comprises the amino acid sequence of SEQ ID NO:48, and a VL that comprises the amino acid sequence of SEQ ID NO:49;
(e) a VH that comprises the amino acid sequence of SEQ ID NO:57, and a VL that comprises the amino acid sequence of SEQ ID NO:58;
(f) a VH that comprises the amino acid sequence of SEQ ID NO:66, and a VL that comprises the amino acid sequence of SEQ ID NO:67;
(g) a VH that comprises the amino acid sequence of SEQ ID NO:75, and a VL that comprises the amino acid sequence of SEQ ID NO:76;
(h) a VH that comprises the amino acid sequence of SEQ ID NO:84, and a VL that comprises the amino acid sequence of SEQ ID NO:85;
(i) a VH that comprises the amino acid sequence of SEQ ID NO:93, and a VL that comprises the amino acid sequence of SEQ ID NO:94;
(j) a VH that comprises the amino acid sequence of SEQ ID NO:102, and a VL that comprises the amino acid sequence of SEQ ID NO:103; or
(k) a VH that comprises the amino acid sequence of SEQ ID NO: 111, and a VL that comprises the amino acid sequence of SEQ ID NO:112.

In some embodiments, the VH and VL of the CD3-binding domain are joined by a linker to form a single chain Fv (scFv). In some embodiments, the linker is a peptide linker comprising a sequence selected from any one of SEQ ID Nos. 114-122. In some embodiments, the linker is a GS liker, such as Gly-Gly-Gly-Gly-Ser (G4S, SEQ ID NO: 115), or polymers thereof, i.e. (Gly4Ser)x (SEQ ID NO: 144), where x is an integer of 1 or greater (e.g. 2 or 3) (e.g., SEQ ID Nos. 121, 122).

In certain embodiments, the CD3-binding domain comprises the amino acid sequence of any one of SEQ ID NOs:23, 32, 41, 50, 59, 68, 77, 86, 95, 104, and 113.

In certain embodiments, the EGFRvIII-binding domain and the CD3-binding domain are joined by a linker. In some embodiments, the linker is a peptide linker comprising a sequence selected from any one of SEQ ID Nos. 114-122. In some embodiments, the linker is a GS liker, such as Gly-Gly-Gly-Gly-Ser (G4S, SEQ ID NO: 115), or polymers thereof, i.e. (Gly4Ser)x (SEQ ID NO: 144), where x is an integer of 1 or greater (e.g. 2 or 3) (e.g., SEQ ID Nos. 121, 122).

In certain embodiments, the anti-EGFRvIII agent described herein comprises two domains. The first domain binds EGFRvIII (preferably human EGFRvIII) and comprises: (a) a heavy chain variable region (VH) that comprises: (i) a VH complementarity determining region one (CDR-H1) comprising the amino acid sequence of SEQ ID NO:3; (ii) a CDR-H2 comprising the amino acid sequence of SEQ ID NO:4; and (iii) a CDR-H3 comprising the amino acid sequence of SEQ ID NO:5; and (b) a light chain variable region (VL) that comprises: (i) a VL complementarity determining region one (CDR-11) comprising the amino acid sequence of SEQ ID NO:6; (ii) a CDR-L2 comprising the amino acid sequence of SEQ ID NO:7; and (iii) a CDR-L3 comprising the amino acid sequence of SEQ ID NO:8. The second domain binds CD3 (preferably human CD3) and comprises: (a) a heavy chain variable region (VH) that comprises: (i) a VH complementarity determining region one (CDR-H1) comprising the amino acid sequence of SEQ ID NO:99; (ii) a CDR-H2 comprising the amino acid sequence of SEQ ID NO:100; and (iii) a CDR-H3 comprising the amino acid sequence of SEQ ID NO:101; and (b) a light chain variable region (VL) that comprises: (i) a VL complementarity determining region one (CDR-L1) comprising the amino acid sequence of SEQ ID NO:96; (ii) a CDR-L2 comprising the amino acid sequence of SEQ ID NO:97; and (iii) a CDR-L3 comprising the amino acid sequence of SEQ ID NO:98.

In certain embodiments, the anti-EGFRvIII agent described herein comprises two domains: (a) the first domain binds EGFRvIII (preferably human EGFRvIII) and comprises: a VH that comprises the amino acid sequence of SEQ ID NO:9, and a VL that comprises the amino acid sequence of SEQ ID NO:10; and (b) the second domain binds CD3 (preferably human CD3) and comprises: a VH that comprises the amino acid sequence of SEQ ID NO:102, and a VL that comprises the amino acid sequence of SEQ ID NO:103.

In certain embodiments, the anti-EGFRvIII agent described herein comprises two domains: (a) the first domain binds EGFRvIII (preferably human EGFRvIII) and comprises the amino acid sequence of SEQ ID NO:11; and (b) the second domain binds CD3 (preferably human CD3) and comprises the amino acid sequence of SEQ ID NO:104.

In certain embodiments, the anti-EGFRvIII agent described herein comprises the amino acid sequence of SEQ ID NO: 12. In certain embodiments, the anti-EGFRvIII agent described herein comprises the amino acid sequence of SEQ ID NO: 13.

Preferably, the anti-EGFRvIII agent is administered parenterally (e.g., intravenously) and then can cross the blood brain barrier (BBB). Without wishing to be bound by a particular theory, it is believed that the binding of CD3 contributes the penetration of BBB by the exemplary anti-EGFRvIII agents described herein. Activated T lymphocytes are known to have the ability to penetrate the BBB under normal physiological conditions. By binding to CD3 on the surface of T cell, it is believed that the exemplary anti-EGFRvIII agents can activate peripheral circulating T cells, thereby passing through the BBB via these T cells.

### 4. Dosing of Anti-EGFRvIII Agents

Disclosed herein are methods of treating glioblastoma (GBM), comprising administering to a subject in need thereof an anti-EGFRvIII agent, at an initial dose of from about 15 μg/day to about 12000 μg/day. Also disclosed herein are methods of treating EGFRvIII-positive cancer, comprising administering to a subject in need thereof an anti-EGFRvIII agent, at an initial dose of from about 15 μg/day to about 12000 μg/day.

The expression of EGFRvIII has been associated with glioblastoma in particular but is also described in a number of cancers, especially solid tumors, such as prostate cancer, head and neck cancer (e.g., HNSCC), lung cancer (e.g., non-small cell lung cancer), brain cancer (e.g., glioma, oligodendroglioma), breast cancer, colorectal cancer, esophageal cancer, adenocarcinoma, squamous cell cancer (SCC), large-cell carcinomas, melanoma, ovarian cancer, peripheral nerve sheath tumor (PNST), sarcoma (e.g., synovial sarcoma), malignant fibro histiocytoma (MFH), osteosarcoma, testicular seminoma, thyroid cancer (e.g., papillary thyroid cancer, follicular thyroid cancer), and other EGFRvIII-positive cancers. See, Gan et al., FEBS Journal, 280(2013) 5350-5370.

In some embodiments, the anti-EGFRvIII agent is administered at an initial dose of: from about 15 μg/day to about 12000 μg/day, from about 15 μg/day to about 11000 μg/day, from about 15 μg/day to about 10000 μg/day, from about 15 μg/day to about 9000 μg/day, from about 15 μg/day to about 8000 μg/day, from about 15 μg/day to about 7000 μg/day, from about 15 μg/day to about 6000 μg/day, from about 15 μg/day to about 5000 μg/day, from about 45 μg/day to about 12000 μg/day, from about 45 μg/day to about 11000 μg/day, from about 45 μg/day to about 10000 μg/day, from about 45 μg/day to about 9000 μg/day, from about 45 μg/day to about 8000 μg/day, from about 45 μg/day to about 7000 μg/day, from about 45 μg/day to about 6000 μg/day, from about 45 μg/day to about 5000 μg/day, from about 150 μg/day to about 12000 μg/day, from about 150 μg/day to about 11000 μg/day, from about 150 μg/day to about 10000 μg/day, from about 150 μg/day to about 9000 μg/day, from about 150 μg/day to about 8000 μg/day, or from about 150 μg/day to about 7000 μg/day, from about 150 μg/day to about 6000 μg/day, from about 150 μg/day to about 5000 μg/day, from about 500 μg/day to about 12000 μg/day, from about 500 μg/day to about 11000 μg/day, from about 500 μg/day to about 10000 μg/day, from about 500 μg/day to about 9000 μg/day, from about 500 μg/day to about 8000 μg/day, from about 500 μg/day to about 7000 μg/day, from about 500 μg/day to about 6000 μg/day, from about 500 μg/day to about 5000 μg/day, from about 1000 μg/day to about 12000 μg/day, from about 1000 μg/day to about 11000 μg/day, from about 1000 μg/day to about 10000 μg/day, from about 1000 μg/day to about 9000 μg/day, from about 1000 μg/day to about 8000 μg/day, from about 1000 μg/day to about 7000 μg/day, from about 1000 μg/day to about 6000 μg/day, from about 1000 μg/day to about 5000 μg/day, from about 1500 μg/day to about 12000 μg/day, from about 1500 μg/day to about 11000 μg/day, from about 1500 μg/day to about 10000 μg/day, from about 1500 μg/day to about 9000 μg/day, from about 1500 μg/day to about 8000 μg/day, from about 1500 μg/day to about 7000 μg/day, from about 1500 μg/day to about 6000 μg/day, from about 1500 μg/day to about 5000 μg/day, from about 2000 μg/day to about 12000 μg/day, from about 2000 μg/day to about 11000 μg/day, from about 2000 μg/day to about 10000 μg/day, from about 2000 μg/day to about 9000 μg/day, from about 2000 μg/day to about 8000 μg/day, from about 2000 μg/day to about 7000 μg/day, from about 2000 μg/day to about 6000 μg/day, from about 2000 μg/day to about 5000 μg/day, from about 3000 μg/day to about 12000 μg/day, from about 3000 μg/day to about 11000 μg/day, from about 3000 μg/day to about 10000 μg/day, from about 3000 μg/day to about 9000 μg/day, from about 3000 μg/day to about 8000 μg/day, from about 3000 μg/day to about 7000 μg/day, from about 3000 μg/day to about 6000 μg/day, or from about 3000 μg/day to about 5000 μg/day.

As shown in the Examples, AMG 596 doses as low as 15 μg/day (see examples 5, 6, and 7) provide steady state exposure above 2-5 ng/mL. Therefore, it is believed that efficacious dose range can be from about 15 μg/day to about 6000 μg/day. To achieve objective antitumor responses as early as after a first treatment cycle, efficacious dose can range from about 1000 μg/day to about 6000 μg/day, or from about 1500 μg/day to about 6000 μg/day.

In some embodiments, the anti-EGFRvIII agent is administered at an initial dose, and one or more subsequent doses of from about 15 μg/day to about 12000 μg/day. For example, the one or more subsequent doses can be as follows: from about 15 μg/day to about 12000 μg/day, from about 15 μg/day to about 11000 μg/day, from about 15 μg/day to about 10000 μg/day, from about 15 μg/day to about 9000 μg/day, from about 15 μg/day to about 8000 μg/day, from about 15 μg/day to about 7000 μg/day, from about 15 μg/day to about 6000 μg/day, from about 15 μg/day to about 5000 μg/day, from about 45 μg/day to about 12000 μg/day, from about 45 μg/day to about 11000 μg/day, from about 45 μg/day to about 10000 μg/day, from about 45 μg/day to about 9000 μg/day, from about 45 μg/day to about 8000 μg/day, from about 45 μg/day to about 7000 μg/day, from about 45 μg/day to about 6000 μg/day, from about 45 μg/day to about 5000 μg/day, from about 150 μg/day to about 12000 μg/day, from about 150 μg/day to about 11000 μg/day, from about 150 μg/day to about 10000 μg/day, from about 150 μg/day to about 9000 μg/day, from about 150 μg/day to about 8000 μg/day, or from about 150 μg/day to about 7000 μg/day, from about 150 μg/day to about 6000 μg/day, from about 150 μg/day to about 5000 μg/day, from about 500 μg/day to about 12000 μg/day, from about 500 μg/day to about 11000 μg/day, from about 500 μg/day to about 10000 μg/day, from about 500 μg/day to about 9000 μg/day, from about 500 μg/day to about 8000 μg/day, from about 500 μg/day to about 7000 μg/day, from about 500 μg/day to about 6000 μg/day, from about 500 μg/day to about 5000 μg/day, from about 1000 μg/day to about 12000 μg/day, from about 1000 μg/day to about 11000 μg/day, from about 1000 μg/day to about 10000 μg/day, from about 1000 μg/day to about 9000 μg/day, from about 1000 μg/day to about 8000 μg/day, from about 1000 μg/day to about 7000 μg/day, from about 1000 μg/day to about 6000 μg/day, from about 1000 μg/day to about 5000 μg/day, from about 1500 μg/day to about 12000 μg/day, from about 1500 μg/day to about 11000 μg/day, from about 1500 μg/day to about 10000 μg/day, from about 1500 μg/day to about 9000 μg/day, from about 1500 μg/day to about 8000 μg/day, from about 1500 μg/day to about 7000 μg/day, from about 1500 μg/day to about 6000 μg/day, from about 1500 μg/day to about 5000 μg/day, from about 2000 μg/day to about 12000 μg/day, from about 2000 μg/day to about 11000 μg/day, from about 2000 μg/day to about 10000 μg/day, from about 2000 μg/day to about 9000 μg/day, from about 2000 μg/day to about 8000 μg/day, from about 2000 μg/day to about 7000 μg/day, from about 2000 μg/day to about 6000 μg/day, from about 2000 μg/day to about 5000 μg/day, from about 3000 μg/day to about 12000 μg/day, from about 3000 μg/day to about 11000 μg/day, from about 3000 μg/day to about 10000 μg/day, from about 3000 μg/day to about 9000 μg/day, from about 3000 μg/day to about 8000 μg/day, from about 3000 μg/day to about 7000 μg/day, from about 3000 μg/day to about 6000 μg/day, or from about 3000 μg/day to about 5000 μg/day.

The anti-EGFRvIII agent can be administered by any suitable means, including parenteral, topical, subcutaneous, intraperitoneal, intrapulmonary, intranasal, and/or intralesional administration. Parenteral administration includes intramuscular, intravenous, intraarterial, intraperitoneal, or subcutaneous administration. Intrathecal administration is also contemplated. In addition, the anti-EGFRvIII agent may be administered by pulse infusion, e.g., with declining doses of the anti-EGFRvIII agent. In some embodiments, the dosing is given intravenously, subcutaneously or intrathecally. In some embodiments, the anti-EGFRvIII agent is administered by intravenous (IV) infusion, such as continuous IV fusion.

In some embodiments, the one or more subsequent doses are administered at least one week after the initial dose, or at least two weeks after the initial dose.

In some embodiments, the initial dose, or one more subsequence doses, are administered for at least 7 days, at least 14 days, at least 21 days, or at least 28 days.

In some embodiments, the subsequent dose is provided between about 1 and about 12 weeks after the previous dose. In some embodiments, the subsequent doses are given between about 2 and about 12 weeks apart. In some embodiments, the subsequent doses are given between about 2 and about 8 weeks apart. In some embodiments, the subsequent doses are given between about 2 and about 6 weeks apart. In some embodiments, the subsequent doses are given between about 2 and about 4 weeks apart. In some embodiments, the subsequent doses are given about 2 weeks apart. In some embodiments, the subsequent doses are given between about 1 and about 3 months apart. In some embodiments, the subsequent doses are given about 1 month apart. In some embodiments, the subsequent doses are given about 2 months apart.

In some embodiments, the invention further provides for the administration of a subsequent dose of the anti-EGFRvIII agent in an amount that is approximately the same or less than the initial dose.

In some embodiments, the anti-EGFRvIII agent is administered at a 7-day on/7-day off cycle, 14-day on/7-day off cycle, or 14-day on/14-day off cycle, 21-day on/7-day off cycle, 21-day on/14-day off cycle, 28-day on/7-day off cycle, or 28-day on/14-day off cycle.

In some embodiments, the compositions and methods of the invention provide for the use of an anti-EGFRvIII agent in combination with one or more additional therapeutic agents.

In some embodiments, the one or more additional therapeutic agent may be an anti-inflammatory agent (for example, to prophylactically treat cerebral edema). The anti-inflammatory agent may be administered prior to, concurrently, or after the administration of the anti-EGFRvIII agent. Exemplary anti-inflammatory agent includes acetaminophen, naproxen sodium, ibuprofen, tramadol, aspirin, celecoxib, valdecoxib, indomethacin, or other NSAIDs. Other anti-inflammatory agent includes, e.g., beclomethasone, hydroxycortisone, betamethasone, methylprednisolone, budesonide, prednisolone, cortisone, prednisone, dexamethasone, and triamcinolone, or other glucocorticoids.

In some embodiments, the one or more additional therapeutic agent may be 6-mercaptopurine, tacrolimus, azathioprine, thalidomide, cyclosporine, tofacitinib, methotrexate, and other immunosuppressants/immunomodulators.

In some embodiments, the one or more additional therapeutic agent is a VEGFR targeting agent, such as bevacizumab, sunitinib, sorafenib, or fluoro-sorafenib (regorafanib).

In some embodiments, the one or more additional therapeutic agent is a steroid. Steroids decrease the permeability of capillaries and the hemato-encephalic barrier, promoting the movement of Na(+)/K(+) ions and water through the main endothelial membrane, and therefore they are used in the treatment of vasogenic cerebral edema as well as edema caused by a cerebral tumor. In an exemplary embodiment, the one or more agent is a corticoid. In an exemplary embodiment, the one or more agent is dexamethasone.

When a steroid (such as dexamethasone) is used, higher doses of anti-EGFRvIII agent may be needed. Accordingly, in some aspect, the invention provides a method of treating glioblastoma, or an EGFRvIII-positive cancer, comprising administering to a subject in need thereof a steroid (such as a corticosteroid, e.g., dexamethasone), and an anti-EGFRvIII agent, wherein said anti-EGFRvIII agent is administered at an initial dose of from about 150 μg/day to about 12000 μg/day (such as from about 1000 μg/day to about 12000 μg/day, from about 1500 μg/day to about 12000 μg/day, from about 2000 μg/day to about 12000 μg/day, from about 3000 μg/day to about 12000 μg/day, or any other dose ranges disclosed above). One or more subsequent dose of anti-EGFRvIII agent may be administered at a dose of from about 150 μg/day to about 12000 μg/day (such as from about 1000 μg/day to about 12000 μg/day, from about 1500 μg/day to about 12000 μg/day, from about 2000 μg/day to about 12000 μg/day, from about 3000 μg/day to about 12000 μg/day, or any other dose ranges disclosed above).

### 5. EGFRvIII Expression and Diagnostics

Also disclosed herein are methods and diagnostic kits for assessing the expression of EGFRvIII. The presence of EGFRvIII, or the expression level of EGFRvIII can assessed by presence of the mutated DNA or mRNA sequence, presence of EGFRvIII protein, cfDNA level, mRNA expression level, protein expression level, activity level, or other quantity reflected in or derivable from the gene or protein expression data. Commonly used methods include, e.g., Immunohistochemistry (IHC), Fluorescence in situ hybridization (FISH), PCR, RT-PCR, and next-generation sequencing (NGS), to detect the presence of EGFRvIII DNA, EGFRvIII RNA or EGFRvIII protein.

In certain embodiments, immunohistochemistry (IHC) is used to assess the presence of EGFRvIII or the expression level of EGFRvIII. Other antibody-based techniques, such as immunoblotting (western blotting), immunohistological assay, enzyme linked immunosorbent assay (ELISA), radioimmunoassay (RIA), Fluorescence in situ hybridization (FISH), or protein chips, may also be used.

The presence of EGFRvIII or the expression level of EGFRvIII may be assessed in a quantitative form (e.g., a number, ratio, percentage, graph, etc.) or a qualitative form (e.g. positive staining or blot, etc.). Quantitatively, a scoring system may be used to assist in determining the EGFR expression levels in tumor samples. For example, the H-score method assigns a score of 0-300 to each sample, based on the percentage of tumor cells stained at different intensities viewed at various magnifications. Previously, the FLEX study assessed EGFR expression using an IHC scoring system according to the intensity of cell membrane staining (scale of 0-3) (Pirker et al., Lancet Oncol. 2012; 13:33-42). The EGFR expression data were used to generate IHC scores on a continuous scale of 0-300. In Examples, H-scores are assigned according to four categories: 0 for ‘no staining’, 1+ for ‘light staining visible only at high magnification’, 2+ for ‘intermediate staining’ and 3+ for ‘dark staining of linear membrane. The percentage of cells at different staining intensities was determined by visual assessment, with the score calculated using the formula: 1×(% of 1+ cells)+2×(% of 2+ cells)+3×(% of 3 cells).

In some embodiments, the H-score of the biological sample of the subject in need of treatment is from about 5 to about 300, from about 8 to about 300, from about 8 to about 295, from about 8 to about 290, from about 8 to about 285, or from about 8 to about 280.

Also included is an assay kit for the detection of EGFRvIII in mammalian tissues or cells in order to screen for EGFRvIII-positive cancers. The kit may comprise a first antibody (“primary antibody”) that binds EGFRvIII, and means for detecting the binding of the primary antibody to EGFRvIII. The first antibody can be a labeled monoclonal antibody, with a detectable label attached. Or the first antibody can be an unlabeled primary antibody, and the means for detecting the binding of the primary antibody to EGFRvIII may be a labeled secondary antibody that binds to an immunoglobulin (such as secondary antibodies that bind to the constant region of immunoglobulin).

The detectable label can be a chemical moiety that can be detected (e.g., imaged) by a standard procedure known to a skilled artisan (such as enzymatic, biochemical, spectroscopic, photochemical, immunochemical, isotopic, electrical, optical, chemical or other means).

Exemplary detectable labels include contrast agents (e.g., gadolinium; manganese; barium sulfate; an iodinated or noniodinated agent; a zirconium-labelled agent, an ionic agent or nonionic agent); electron-dense, magnetic and paramagnetic reagents, labels or agents (e.g., iron-oxide chelate); nanoparticles; an enzyme (horseradish peroxidase (HRP), urease, catalase, alkaline phosphatase, 3-galactosidase, chloramphenicol transferase or acetylcholinesterase); a prosthetic group or ligand (e.g., biotin, streptavidin/biotin and avidin/biotin); a colorimetric label such as colloidal gold or colored glass or plastic (e.g., polystyrene, polypropylene, latex, etc.) beads; a fluorescent material, dye and fluorophore (e.g., allophycocyanin, umbelliferone, fluorescein, fluorescein isothiocyanate, fluorscamine, rhodamine, dichlorotriazinylamine fluorescein, dansyl chloride, texas red, phycoerythrin phycocyanin); a chemiluminescent or a bioluminescent material (e.g., imidazole, acridinium, oxalate, luminol, luciferase, luciferin, aequorin). A detectable label can also be any imaging agent that can be employed for detection, measurement, analysis, monitoring, and/or quantitation (e.g., for computed axial tomography (CAT or CT), fluoroscopy, single photon emission computed tomography (SPECT) imaging, optical imaging, positron emission tomography (PET), magnetic resonance imaging (MRI), gamma imaging).

Further exemplary detectable labels include a radioactive material, such as a radioisotope, a metal or a metal oxide. A tag can also be linked or attached to an antibody, such as His-tag or FLAG-tag.

An exemplary antibody, or antigen-binding fragment thereof, for assessing the presence of EGFRvIII, or the expression level of EGFRvIII (“EGFRvIII antibody 1”) is provided as SEQ ID Nos: 127-138. The six CDRs are shown as SEQ ID Nos: 127-132. The VH and VL portion of this antibody is derived from human, and are shown as SEQ ID NO: 133 and 134. It is known in the art that amino acid residues often can be modified in the framework region without significantly impact the binding of the antibody. Therefore, VH and VL sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO:133 or SEQ ID NO:134 may be used.

The constant regions of this exemplary antibody are derived from murine IgG1/kappa. While constant regions of the primary antibody can be either human or murine, when examine human tissue samples, murine constant regions are preferred because secondary antibodies used for detection will then only recognize murine immunoglobulin constant regions. This can reduce the cross-reaction or false positives. Endogenous antibodies within the tissue sample would not interfere with appropriate detection of the primary antibody.

In-house data have shown that certain anti-EGFRvIII antibodies may cross-react with human skin tissues. For example, a second anti-EGFRvIII antibody (EGFRvIII antibody 2, see, SEQ ID Nos. 139-142) showed some possible rare to occasional cross reactivity with sweat ducts/glands in some skin sample (staining photographs not shown). This second antibody has the same CDR sequences as the CDRs of the EGFRvIII-binding domain of AMG 596. The VH region of this second antibody shares about 98% sequence identity with the EGFRvIII VH domain of AMG 596, and the VL region of this second antibody shares about 99% sequence identity with the EGFRvIII VL domain of AMG 596 (compare, SEQ ID NO. 9 vs. SEQ ID NO: 139, and SEQ ID NO. 10 vs. SEQ ID NO: 140). The constant regions of this second antibody are derived from murine for reasons stated above.

A third anti-EGFRvIII antibody tested in-house showed strongly positive staining on normal human skin cells (staining photographs not shown), even though RT-PCR testing of these skin sections confirm that they were negative for EGFRvIII. This third antibody also showed strong cross reactivity with human SCC tumor samples that are believed to be negative for EGFRvIII. Cross-reactivity with human skin was not observed for EGFRvIII antibody 1 (SEQ ID Nos: 127-138). Therefore, testing on EGFRvIII-negative cells might be needed to select for EGFRvIII antibodies that shows low cross-reactivity.

It was further discovered that when the concentration of primary antibody is high, nonspecific staining on human skin and negative cell-line controls may also occur. Therefore, for best results, the concentration of the primary antibody may need to be titrated. In one particular example, it was found that for EGFRvIII antibody 1, incubation at 5.5 μg/ml concentration produced mild nonspecific staining. Two lower concentrations, 2.75 μg/ml & 1.375 μg/ml, resulted in greater specificity on negative controls without significant loss of staining intensity on positive controls. In particular, incubation at 1.375 μg/ml demonstrated a robust signal to noise ratio and superior reproducibility between different experiments. For EGFRvIII antibody 2, reducing the antibody concentration from 0.69 μg/ml to 0.345 μg/ml or 0.1725 μg/ml also reduced some nonspecific staining.

Accordingly, in some embodiment, the invention provides a method for assessing the presence of EGFRvIII, or the expression level of EGFRvIII, comprising: (i) incubating a tissue sample from a subject with a first (primary) antibody that binds to EGFRvIII, wherein said first antibody comprises a VH sequence comprising SEQ ID NO: 133 and a VL sequence comprising SEQ ID NO:134; and wherein the concentration of the first antibody is 3 μg/ml or lower (such as 2.75 μg/ml or 1.375 μg/ml); (ii) removing the excess, unbound first antibody; and (iii) incubating the sample with a second antibody, wherein the second antibody binds to a constant domain of the first antibody. In certain embodiments, the constant domain of the first antibody comprises murine CH (such as murine IgG1 isotype, SEQ ID NO:135). In certain embodiments, the constant domain of the first antibody comprises murine CL (such as murine kappa isotype, SEQ ID NO:136). In certain embodiments, the constant domain of the first antibody comprises a murine CH and a CL (such as murine IgG1 CH and kappa CL, SEQ ID Nos. 135 and 136). In certain embodiments, the first antibody comprises a heavy chain sequence comprising SEQ ID NO:137, and a light chain sequence comprising SEQ ID NO:138. The method may further comprise additional steps to remove the excess, unbound second antibody, and detect the presence of EGFRvIII, or the expression level of EGFRvIII, using a detectable label. The detectable label can be HRP (horseradish peroxidase).

### 6. Articles of Manufacture

Disclosed herein are articles of manufacture comprising: (a) a container comprising an anti-EGFRvIII agent; and (b) a package insert with instructions for treating EGFRvIII-positive cancer (or treating glioblastoma) in a subject, wherein the instructions specifies that an initial dose of from about 15 μg/day to about 12000 μg/day (or any of the dose ranges disclosed herein) of the anti-EGFRvIII be administered to the subject. The instructions may also specify that one or more subsequent doses of from about 15 μg/day to about 12000 μg/day (or any of the dose ranges disclosed herein) of the anti-EGFRvIII agent be administered to the subject. The instructions may also specify that the first, and one more subsequent doses be administered for at least 14 days, at least 21 days, or at least 28 days.

Also disclosed herein are diagnostic kits comprising: (a) a container comprising a first antibody that binds to EGFRvIII; (b) a second antibody that binds to a constant domain of the first antibody; (c) a detectable label; and (d) a package insert with instructions for assessing the presence of EGFRvIII, or the expression level of EGFRvIII.

### EXAMPLES

### Introduction

The urgent medical need for glioblastoma treatment has driven the development of new immunotherapy concepts despite the classic dogma that the central nervous system is immune-privileged and hence inaccessible to potent antitumor immunity. Novel immunotherapeutic concepts have shown success in advanced melanoma, in non-small cell lung cancer and renal cell cancer. Such success also brings new insights into relevant tumor antigens and expression of markers of immune regulation, such as PD-1/PD-L1. Moreover, trafficking of functionally-active T-cells to the central nervous system (CNS) has been demonstrated (Gedeon et al., Expert Rev. Clin. Pharmacol., 2013; 6:375-386). The regression of all intracranial and spinal glioblastoma lesions after multiple intracranial infusions of interleukin 13 receptor alpha 2 targeting CAR T-cell therapy has been reported recently (Brown et al., N Engl J Med., 2016; 375:2561-2569).

AMG 596 is a BiTE® molecule targeting EGFRvIII receptor as a tumor-specific antigen and T-cell receptor-associated complex cluster of differentiation 3 (CD3) on T-cells. AMG 596 is a potent molecule acting by formation of an immunological synapse between CD3+ T-cells and cancer cells expressing the targeted transmembrane protein. T-cell-induced cytotoxicity ensues upon binding to both targets and formation of an immunological synapse. AMG 596 showed high activity in recruiting T-cells against EGFRvIII expressing GBM cell lines in vitro and significantly prolonged survival of systemically treated mice versus control animals (Kischel et al., Eur J Cancer, 2016, 69(Suppl 1):S96; Abstract P117). Furthermore, no direct AMG 596-related adverse changes were observed in a preclinical safety study in cynomolgus monkeys at doses of up to 6.6 mg/kg/day with a large exposure at serum concentrations of up to approximately 21 μg/mL. Therefore, AMG 596 is being investigated for the safety and antitumor activity of T cell mediated immunotherapy in subjects with glioblastoma.

### Example 1 Study Design

Study 20160132 is a phase 1 study to explore escalating doses of AMG 596 in subjects with EGFRvIII-positive glioblastoma or other malignant glioma. The study has 2 parts, dose escalation (Part 1) and dose expansion (Part 2). It enrolls 2 groups of subjects with EGFRvIII-positive glioblastoma according to disease stage: recurrent disease (Group 1), and maintenance treatment after standard of care in newly diagnosed disease (Group 2). The primary objective is to evaluate the safety and tolerability of AMG 596 administered by continuous intravenous (cIV) infusion in subjects with EGFRvIII-positive glioblastoma in the recurrent (Group 1) and maintenance (Group 2) settings. The secondary objectives of this study are to evaluate the PK profile of AMG 596 in serum when administered by cIV infusion, the clinical benefit of AMG 596 as determined by objective response rate (ORR) per modified Response Assessment in Neuro-Oncology Criteria (RANO), the progression free survival rate at 6 and 12 months after initiation of treatment, and the formation and incidence of anti-AMG 596 antibodies.

AMG 596 is delivered through infusion lines using preprogrammed infusion pumps approved for use in the country in which the subject is undergoing treatment. The drug is administered as a cIV infusion at a constant flow rate for 28 days in 28-day on/14-day off cycles, until confirmed disease progression. Pre-specified nominal doses for use in the dose escalation are 15, 45, 150, 500, 1000, 1500, 3000, 6000, 12000 μg/day. Intensive PK samples for cIV 7-day infusion (predose to 192 hours) and cIV 28-day infusion (predose to 696 hours) are collected in this study.

Study 20180427 was created for Subject 13266001006 that was previously enrolled in Study 20160132. In the 20160132 study, the subject was enrolled to Cohort 2b (15 μg/day cIV 28 days on followed by 14 days off). During Cycle 1, days 1-7, the subject received an overdose (1500 μg/day) due to a mixing error by the pharmacy. During Cycle 2, a dose of 15 μg/day was administered to the subject. Because of the significant tumor shrinkage after the first Cycle of therapy on study 20160132, approval was received from the FDA to allow the subject to receive the 1,500 μg/day dose during week one of a treatment Cycle again. Following the approval, subject 13266001006 discontinued from Study 20160132 and continued treatment in study 20180427 as subject 42766001001 to receive the agreed-on treatment (1,500 μg/day during week 1, followed by 15 μg/day during weeks 2-4, followed by a 2-week break). Intensive PK samples for cIV 28-day infusion (predose to 696 hours) were collected in this study.

Table 1 summarizes the Eligibility Criteria for Study 20160132.

TABLE 1
Key Eligibility Criteria

| Key inclusion criteria | Key exclusion criteria: |
|---|---|
| Male orfemale ≥ 18 years of age with EGFRvIII- | Central nervous system (CNS) bleeding: stroke |
| positive Grade IV GBM or lower grade malignant | or intraocular bleed (including embolic stroke) not |
| glioma | associated with antitumor surgery ≤ 6 mos prior |
| | to enrollment |
| Group 1 (rGBM): recurrent disease confirmed by | Unresolved toxicities from prior anti-tumor |
| MRI | therapy: |
| | Lack of resolution to CTCAE v4.0 grade 1 |
| | with exceptions as defined in the study |
| | protocol |
| Group 2 (nGBM): completed SOC therapy, such | Group 1 (rGBM): Antitumor therapy |
| as surgery with adjuvant radiochemotherapy with | (chemotherapy, antibody therapy, molecular |
| or without maintenance TMZ | targeted therapy, or investigational agent) ≤ 14 |
| | days |
| ≤2 mg/day dexamethasone | Group 2 (nGMB): Antitumor therapy |
| | (chemotherapy, antibody therapy, molecular |
| | targeted therapy, or investigational agent) ≤ 14 |
| | days or 5 half-lives of enrollment |
| Eastern Cooperative Oncology Group (ECOG) | |
| performance status ≤ 1 | |
| Life expectancy ≥ 3 mos per the opinion of the | |
| study investigator | |
| Acceptable renal, hematological, and hepatic | |
| function | |

CTCAE = Common Terminology Criteria for Adverse Events;
SOC = standard of care;
TMZ = temozolomide

### Example 2: Study Endpoints

There are two clinical hypotheses for this Phase 1 study. First, AMG 596 is safe and well tolerated in at least one dose level when administered in subjects with EGFRvIII-positive glioblastoma or malignant glioma in the recurrent (Group 1) and thereafter in the maintenance setting (Group 2). Two, AMG 596 can induce objective tumor shrinkage and/or overcome lack of response to standard of care (SoC) in subjects with EGFRvIII-positive glioblastoma or malignant glioma in either recurrent or in the maintenance setting at a tolerable dose.

Primary Endpoint (Safety Endpoint): evaluation of safety and tolerability of AMG 596, with the frequency of the following parameters being assessed: dose limiting toxicities (DLT), treatment-emergent adverse events, treatment-related adverse events and clinically significant changes in vital signs, physical examinations, and clinical laboratory tests.

Secondary Endpoints (Efficacy Endpoints): (1) proportion of subjects with Objective response (OR) as per modified RANO (see below), assessment of time to response, response duration and time to progression (TTP); and (2) proportion of subjects with Progression free survival (PFS) at 6 and 12 months after treatment initiation.

Secondary Endpoint (PK Endpoint): PK parameters for AMG 596 including, but not limited to, average steady-state concentration (Cₛₛ), area under the concentration-time curve (AUC), clearance, volume of distribution and half-life (t_{1/2}) for serum AMG 596.

Exploratory Endpoints (Pharmacodynamic Endpoints): (i) concentration-time profiles of AMG 596 in cerebral spinal fluid (CSF); (ii) immune cell counts and immunological marker expression in blood, CSF and tissue; (iii) levels of EGFRvIII expression at protein, RNA, and DNA levels; (iv) genetic mutations relevant to EGFRvIII signaling; (v) anti-AMG 596 antibody formation.

Modified Response Assessment In Neuro-Oncology (RANO). The RANO criteria are extensions to the Macdonald criteria that incorporate T2/FLAIR images to better capture lesion response (Wen et al, J Clin Oncol. 2010, 28(11):1963-72). Here, the RANO criteria are further modified to capture pseudo-progression and delayed responses which may be observed in response to immunotherapies (Okada et al, Lancet Oncol 2015; 16(15): e534-542). Definitions are: (1) measurable lesions contrast-enhancing lesions that can accurately be measured bidimensionally with ≥10 mm longest diameter and ≥10 mm perpendicular diameter and noted on more than one imaging slice; (2) non-measurable lesions—all other lesions, including small lesions, i.e., bone lesions, leptomeningeal disease and cystic lesions that are not confirmed and followed by imaging techniques.

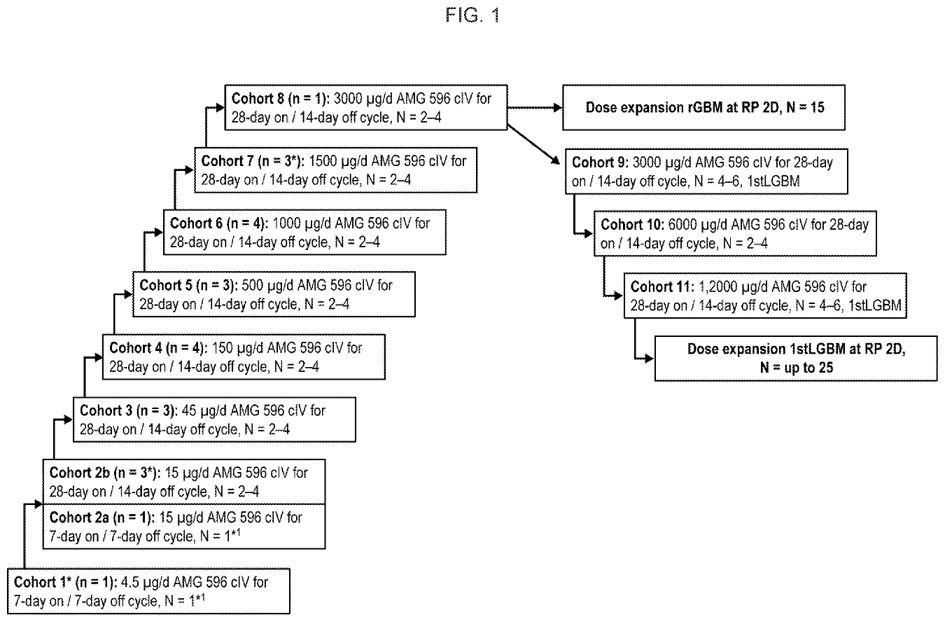

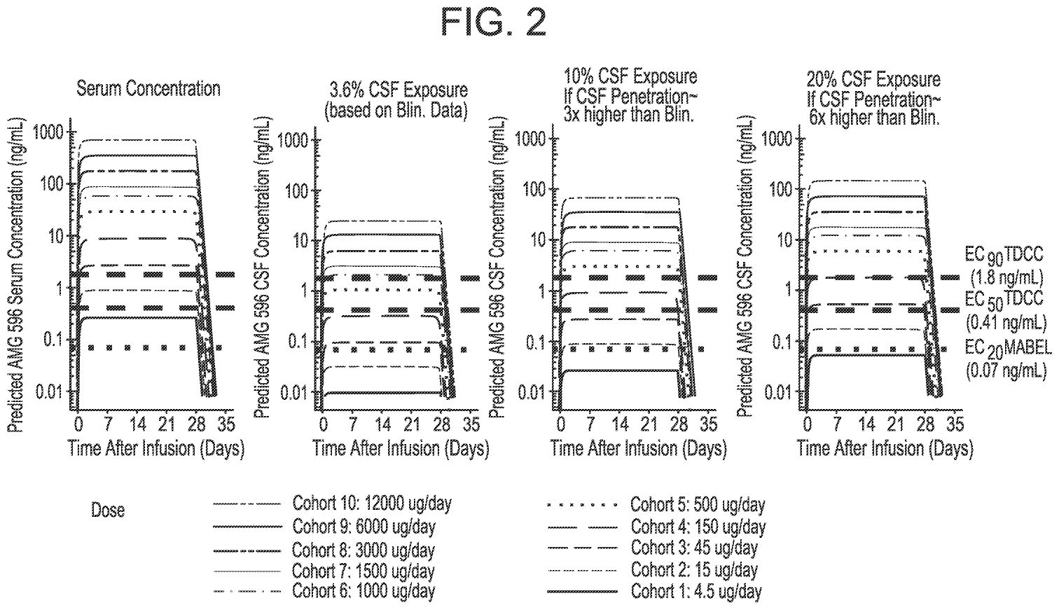

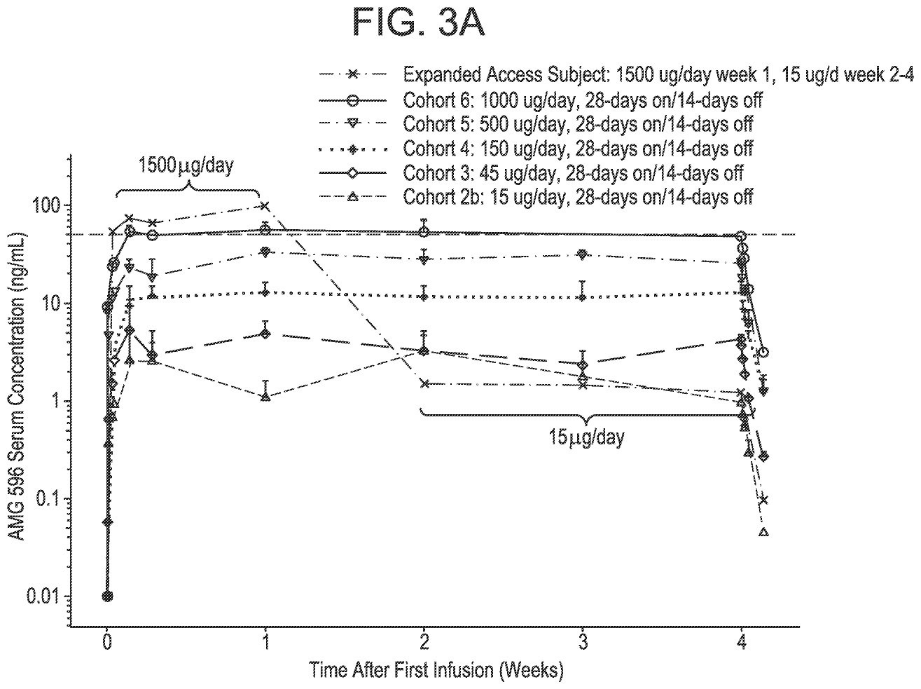

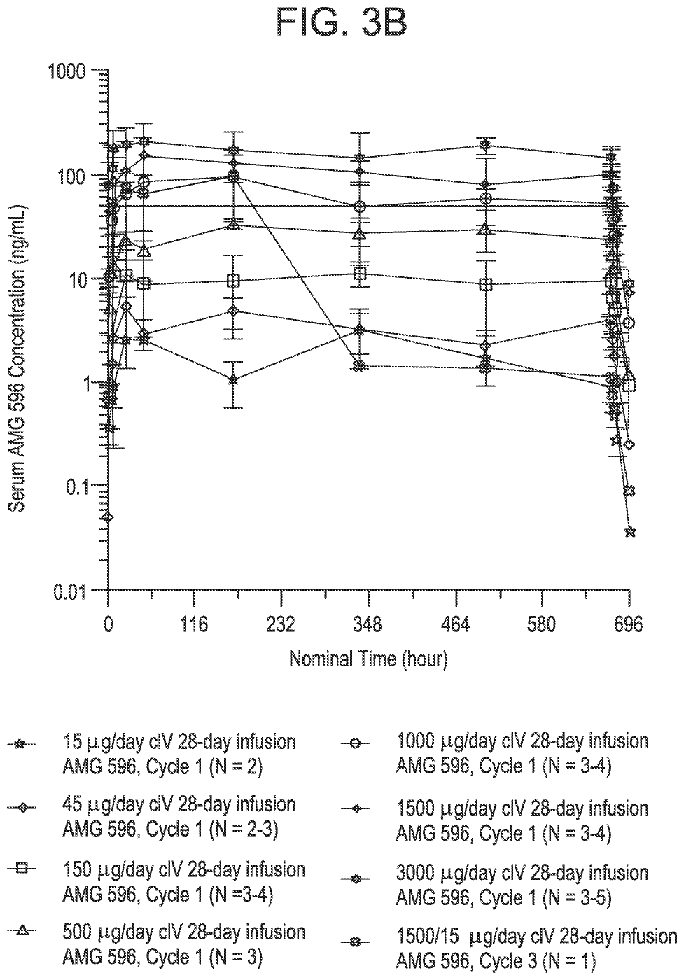

In general, dose-escalation proceeds according to the pre-planned nominal doses, though intermediate dose levels may be used if required after reviewing all available safety data. When a first DLT is observed, the Bayesian logistic regression model (BLRM) will be used to guide dose level selection (Neuenschwander et al., Stat Med 2008; 27(13): 2420-2439). The cohort size is N=2-4 subjects. On a limited basis, one additional subject may be allowed to be enrolled. After each cohort, the model's recommended Maximum Tolerated Dose (MTD) dose level for evaluation will be the dose level with the highest probability of the target toxicity probability interval (TPI), but with a less than 0.25 probability of an excessive or unacceptable TPI. The target TPI is (0.20, 0.33], and TPIs of (0.33, 0.60] and (0.60, 1.00] are defined as excessive and unacceptable, respectively. The actual dose selected at each dose decision may be at or below the model's recommended dose. The study scheme is shown in FIG. 1.

### Example 3: Endpoint Analysis

Safety data after each cohort are reviewed in order to make a decision on the next dose level to be explored, and on the estimate of Recommended Phase 2 Dose (RP2D)/MTD based on a BLRM design. The RP2D and MTD are established separately for Group 1 (recurrent disease) and Group 2 (maintenance setting) subjects. The interim analysis includes the establishment of RP2D/MTD and the estimate of ORR first for subjects with recurrent disease.

The first interim of safety data analysis in Part 1 dose escalation happens at the earlier of: (1) when 15 subjects enrolled and completed DLT observation, or (2) completion of dose escalation of Group 1. Efficacy data are also analyzed for subjects who have had at least one imaging evaluation after start of treatment or have dropped out before that.

Since dose escalation was not completed at the time point of the first interim analysis, safety and efficacy information derived from subjects treated during or after the interim analysis are assessed as well.

### Example 4: Demographic Data and Subject Disposition

4a. Demographic Data and Subject Disposition as of 1 Jul. 2019

Sixteen subjects were analyzed in the interim analysis. One subject never received therapy, 8 subjects were female, and 7 subjects were male, the median age was 55 years (range, 44 to 69) with 2 subjects being older than 65 years. A performance status of Eastern Cooperative Oncology Group (ECOG) 0 was reported for 4 (26.7%) subjects and 11 (73.3%) subjects presented with ECOG 1 at study entry. Glioblastoma was diagnosed in 14 subjects at initial diagnosis. The tumor was shown to be positive for EGFRvIII in at least one test. All subjects underwent prior surgery and had received radiotherapy and other anti-cancer therapies. At time point of the interim analysis, 10 (62.5%) subjects had stopped AMG 596 treatment due to disease progression and 5 (31.3%) subjects were ongoing in AMG 596 treatment. AMG 596 treatment included one subject at a dose of 4.5 ug per day, 4 subjects at 15 ug per day, 3 subjects at 45 ug per day, 4 subjects at 150 ug per day and 4 subjects at 500 ug per day. One subject of the 15 ug per day cohort received an AMG 596 dose of 1500 ug per day during week one of cycle 1. One subject of the 15 ug per day cohort was escalated to 500 ug per day starting with cycle 9. One subject of the 45 ug per day cohort was escalated to 150 ug per day starting cycle 5 and further escalated to 1000 ug per day starting cycle 7.

Moreover, 4 subjects (m/f is 3/1; age 49, 55, 55 and 61 years) started treatment at 1000 ug and 2 subjects at 1500 ug (m/f is 1/1 and age is 34 and 62 years). Table 2 summarizes Key baseline characteristics.

TABLE 2
Key baseline characteristics were generally similar across cohorts

| | 4.5 μg/d | | 15 μg/d | | 45 μg/d | | 150 μg/d | | 500 μg/d | | 1000 μg/d | | 1500/15 μg/d | | All | |
|---|---|---|---|---|---|---|---|---|---|---|---|---|---|---|---|---|
| | (n = 1) | | (n = 3) | | (n = 3) | | (n = 4) | | (n = 3) | | (N = 4) | | (n = 1) | | (N = 19) | |
| Sex, n (%) | | | | | | | | | | | | | | | | |
| Male | — | | 1 | (33.3) | 3 | (100.0) | 2 | (50.0) | 2 | (66.7) | 3 | (75.0) | — | | 11 | (57.9) |
| Female | 1 | (100.0) | 2 | (66.7) | — | | 2 | (50.0) | 1 | (33.3) | 1 | (25.0) | 1 | (100.0) | 8 | (42.1) |
| Median | 62.0 | (62-62) | 54.0 | (44-55) | 59.0 | (47-64) | 57.0 | (50-68) | 54.0 | (46-69) | 55.0 | (49-61) | 49.0 | (49-49) | 55.0 | (44-69) |
| age,yr | | | | | | | | | | | | | | | | |
| (range) | | | | | | | | | | | | | | | | |
| ECOG | | | | | | | | | | | | | | | | |
| per- | | | | | | | | | | | | | | | | |
| formance | | | | | | | | | | | | | | | | |
| status,* | | | | | | | | | | | | | | | | |
| n (%) | | | | | | | | | | | | | | | | |
| 0 | — | | 2 | (66.7) | 2 | (66.7) | — | | — | | 2 | (50.0) | — | | 6 | (31.6) |
| 1 | 1 | (100.0) | 1 | (33.3) | 1 | (33.3) | 4 | (100.0) | 3 | (100.0) | 2 | (50.0) | 1 | (100.0) | 13 | (68.4) |
| Primary | | | | | | | | | | | | | | | | |
| tumor | | | | | | | | | | | | | | | | |
| type at | | | | | | | | | | | | | | | | |
| diagnosis, | | | | | | | | | | | | | | | | |
| n (%) | | | | | | | | | | | | | | | | |
| Glio- | 1 | (100.0) | 2 | (66.7) | 3 | (100.0) | 4 | (100.0) | 3 | (100.0) | 3 | (75.0) | 1 | (100.0) | 17 | (89.5) |
| blastoma | | | | | | | | | | | | | | | | |
| multiforme | | | | | | | | | | | | | | | | |
| Anaplastic | — | | 1 | (33.3) | — | | — | | — | | — | | — | | 1 | (5.3) |
| astro- | | | | | | | | | | | | | | | | |
| cytoma | | | | | | | | | | | | | | | | |
| Malignant | — | | — | | — | | — | | — | | 1 | (25.0) | — | | 1 | (5.3) |
| glioma | | | | | | | | | | | | | | | | |
| grade | | | | | | | | | | | | | | | | |
| Disease | | | | | | | | | | | | | | | | |
| grade at | | | | | | | | | | | | | | | | |
| diagnosis, | | | | | | | | | | | | | | | | |
| n (%) | | | | | | | | | | | | | | | | |
| Grade II | — | | — | | — | | — | | — | | 1 | (25.0) | — | | 1 | (5.3) |
| Grade III | — | | 1 | (33.3) | — | | — | | — | | — | | — | | 1 | (5.3) |
| Grade IV | 1 | (100.0) | 2 | (66.7) | 3 | (100.0) | 4 | (100.0) | 3 | (100.0) | 3 | (75.0) | 1 | (100.0) | 17 | (89.5) |

| | | | | | | | | | | | | | | | |
|---|---|---|---|---|---|---|---|---|---|---|---|---|---|---|---|
| Tumor | — | 910.0 | (702.0- | 260.0 | (216.0- | 702.0 | (240.0- | 2788.0 | (675.0- | 1077.5 | (336.0- | 418.0 | (418.0- | 806.0 | (216.0- |
| burden, | | | 2035.0) | | 936.0) | | 3600.0) | | 5589.0) | | 2413.0) | | 418.0) | | 5589.0) |
| mm² | | | | | | | | | | | | | | | |
| (range) | | | | | | | | | | | | | | | |
| Re- | | | | | | | | | | | | | | | |

| currences | | | | | | | | | | | | | | |
|---|---|---|---|---|---|---|---|---|---|---|---|---|---|---|
| 1 | — | 3 | (100.0) | — | 1 | (25.0) | 1 | (33.3) | 4 | (100.0) | 1 | (100.0) | 10 | (52.63) |

| 2 | — | | — | 2 | (66.7) | 2 | (50.0) | — | | 4 | (21.1) |
|---|---|---|---|---|---|---|---|---|---|---|---|
| 3 | 1 | (100.0) | — | 1 | (33.3) | 1 | (25.0) | 2 | (66.7) | 5 | (26.3) |

| | | | | | | | | | | |
|---|---|---|---|---|---|---|---|---|---|---|
| Dexa- | 1 | (100.0) | — | — | — | — | — | — | 1 | (5.3) |
| methasone | | | | | | | | | | |

treatment

*0 = fully active; 1 = restricted in physically strenuous activity; 2 = ambulatory and capable of all self-care.
†Patients who received concurrent dexamethasone treatment up to 2 mg/d.
d, day; ECOG PS, Eastern Cooperative Oncology Group performance status; yr, years.

4b. Updated Demographic Data and Patient Disposition as of 13 Aug. 2020

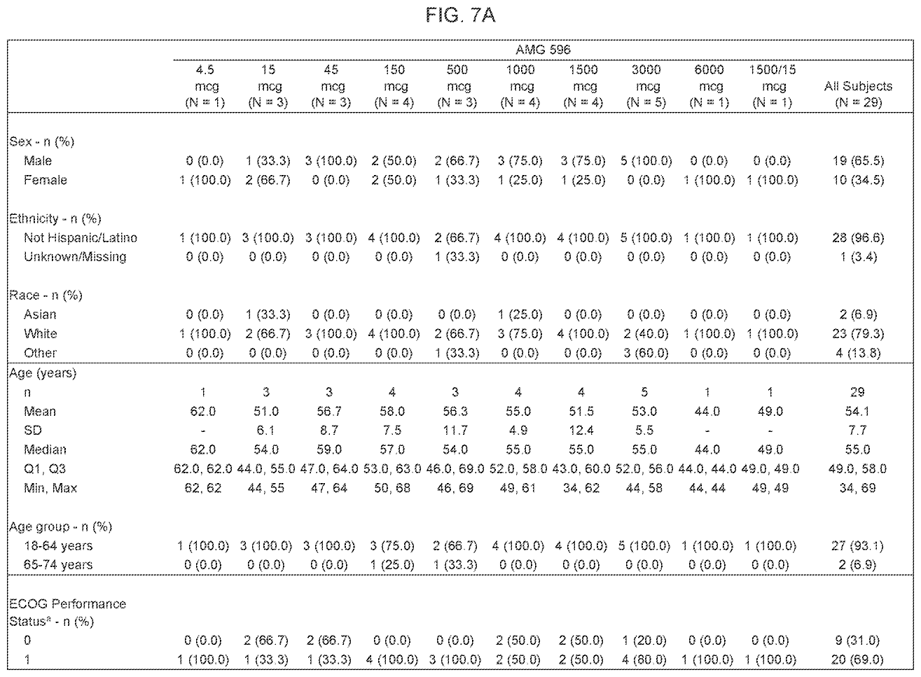

As of cut-off date 13 Aug. 2020, 29 subjects received AMG 596. Of the 29 subjects, 19 subjects (65.5%) were men and 10 subjects (34.5%) were women. Subjects were predominately white (23 subjects [79.3%]), with a median (range) age of 55.0 (34 to 69) years. Most subjects (20 subjects, 69.0%) had an Eastern Cooperative Oncology Group performance status of 1 at baseline. The predominate primary tumor type at initial diagnosis was glioblastoma multiforme in 26 subjects (89.7%), 2 subjects (6.9%) had an initial diagnosis of anaplastic astrocytoma, and 1 subject (3.4%) had other type of tumor at initial diagnosis. The predominate disease grade at initial diagnosis was grade 4 (22 subjects [75.9%]). Before starting this study, all 29 subjects who received treatment had prior surgeries; 28 subjects (96.6%) each had prior antitumor therapy and radiation. FIGS. 7A-7B.

### Example 5: Pharmacokinetic Predictions and Clinical Results

5a. Exposure Predictions

Predictions for AMG 596 concentrations in serum and CSF were done to predict potential efficacious dose ranges. Due to the expected variability in penetration of AMG 596 to the brain tumor the various assumptions included 3.6% CSF exposure, 10% and 20% CSF exposure versus serum concentrations.

Based on in vitro experiments the concentration found to produce 90% cytotoxic activity was selected to mark the lower threshold of the potentially efficacious dose range (1.8 ng/mL).

5b(1). Pharmacokinetic Analyses as of 1 Jul. 2019

PK data analyses were performed on individual serum AMG 596 concentrations to estimate the following PK parameters: (1) the apparent clearance (CL) after continuous IV infusion; (2) the concentration at steady state (Cₛₛ); (3) the terminal half-life (t %, Z) associated with λ_{z}; (4) the apparent volume of distribution (V_{z}) after continuous IV infusion. For study 20160132, Cₛₛ was calculated as 24 to 168 hours for cIV 7-day infusion and 24 to 672 hours for cIV 28-day infusion. For study 20180427, Cₛₛ was calculated for week 1 (24 to 168 hours) and for weeks 2 to 4 (336 to 672 hours).

Concentrations below the lower limit of quantification, LLOQ (0.05 ng/mL) were set to zero before data analysis. All individual PK parameters and descriptive statistics are presented to 3 significant figures except for % CV, which was reported to 1 decimal place.

PK analysis set for study 20160132 on Cycle 1 was comprised of a total of 239 AMG 596 samples from 18 subjects. Of these, 15 samples from subject 13266001006 in the 15 μg/day cIV 28-day infusion group in Cycle 1 were excluded from the PK analysis due to the subject having received the incorrect dose of 1500 μg/day. One sample from subject 13242001041 in the 500 μg/day cIV 28-day infusion group in Cycle 1 day 1, 2 hours timepoint was excluded from the PK analysis due to a duplicate nominal timepoint and according to the PK collection date and time, this sample was most likely an unscheduled sample. Unscheduled samples (n=18) were also not included in the PK analysis.

The PK analysis set for study 20180427 on Cycle 3 was comprised of a total of 12 AMG 596 samples from one subject. There were no exclusions of samples from the PK analysis for study 20180427.

5b(2). Updated PK Analysis as of 13 Aug. 2020

Updated PK analysis set for study 20160132 on Cycle 1 was comprised of a total of 379 AMG 596 samples from 28 subjects. Of these, 15 samples from subject 13266001006 in the 15 μg/day cIV 28-day infusion group in Cycle 1 were excluded from the PK analysis due to the subject having received the incorrect dose of 1500 μg/day. One sample from subject 13242001041 in the 500 μg/day cIV 28-day infusion group in Cycle 1 day 1, 2 hours timepoint was excluded from the PK analysis due to a duplicate nominal timepoint and according to the PK collection date and time, this sample was most likely an unscheduled sample. Unscheduled samples (n=47) were also not included in the PK analysis.

In addition, preliminary PK data analyses were performed on individual CSF AMG 596 concentrations to estimate the serum to CSF penetration. 6 CSF samples from 3 different subjects were available for analysis. The lower limit of quantification, LLOQ for quantifying AMG 596 in the CSF was 0.05 ng/mL. Out of the 6 CSF samples, 1 was taken pre-dose (subject 13226001002, Cohort 3: 45 μg/d), 2 were taken during AMG 596 infusion (subject 13226001002, Cohort 3: 45 μg/d and subject 13266003002 Cohort 7:1500 μg/d), 1 was taken ˜16 hours (subject 13226001002 Cohort 3: 45 μg/d), 1 was taken 2 days (subject 13226001002, Cohort 3: 45 μg/d) and 1 was taken 18 days after the end of AMG 596 infusion (subject 13242001041, Cohort 5: 500 μg/d).

5c(1): Observed Clinical PK Results as of 1 Jul. 2019

Preliminary PK results became available for 16 subjects with recurrent glioblastoma expressing EGFRvIII that received AMG 596 in study 20160132 via continuous infusion.

Following cIV infusion of AMG 596, steady-state was quickly reached after approximately 24 hours. Serum AMG 596 concentrations increased with increasing dose across all cohorts, exposures increased in an approximate dose proportional manner and a rapid elimination was observed for AMG 596.

Preliminary analysis suggests that AMG 596 exposures increased approximately dose proportionally. Observed average steady state concentration (Cₛₛ) in cohorts 1 (4.5 μg/day) to 6 (1000 μg/day) ranged from 0.95 to 52 ng/mL. Steady state was reached within 24 to 48 hours after the start of infusion, which was within the predicted range. The currently available preliminary PK data for dose cohorts 3 to 6 are within ˜2.5-4 fold of predictions. A rapid elimination was observed for AMG 596 with an observed preliminary terminal half-life (t_{1/2}) between ˜6 to 8 hours, which is as expected for a canonical BiTE® molecule.

The potentially efficacious dose range was reached by one subject treated with 1500 mcg per day during week 1 cycle 1. The observed steady state concentration in serum was 79 ng/mL. Assuming 3.6% or 10% or 20% exposure in CSF, a theoretical concentration of 2.84 ng/mL or 7.9 ng/mL or 15.8 ng/mL could be revealed, all being above the assumed lower threshold of 1.8 ng/mL. Table 3A summarizes the observed AMG 596 PK. Cohort 6 (1000 μg/day) exposure levels are within range of projected minimum efficacious exposures and within 2-fold of expanded access subject that showed signs of efficacy after receiving 1500 μg/day overdose. Table 4A shows that nineteen patients received AMG 596 for a median duration of 9 weeks (range 4-52 weeks).

TABLE 3A
Observed average steady state concentration (C_{SS})

| Cohort | Dose (ug/day) | Observed C_{SS} (ng/mL) | N |
|---|---|---|---|
| 1 | 4.5 | 0.95 | 1 |
| 2a/2b | 15 | 2.0 | 3 |
| 3 | 45 | 3.6 | 3 |
| 4 | 150 | 12 | 4 |
| 5 | 500 | 26 | 3 |
| 6 | 1000 | 52 | 2† |
| Overdose | 1500 | 79 | 1‡ |
| 7 | 1500 | | |

TABLE 4A
AMG 596 Exposure: nineteen patients received AMG 596 for a median duration of 9 weeks (range 4-52 weeks)

| | 4.5 μg/d | | 15 μg/d | | 45 μg/d | | 150 μg/d | | 500 μg/d | | 1000 μg/d | | 1500/15 μg/d | | All | |
|---|---|---|---|---|---|---|---|---|---|---|---|---|---|---|---|---|
| | (N = 1) | | (N = 3) | | (N = 3) | | (N = 4) | | (N = 3) | | (N = 4) | | (N = 1) | | (N = 19) | |
| Median | 9.0 | (9-9) | 10.0 | (9-52) | 11.0 | (4-34) | 11.0 | (10-16) | 8.0 | (4-9) | 4.0 | (4-8) | 10.0 | (10-10) | 9.0 | (4-52) |
| duration, | | | | | | | | | | | | | | | | |
| wk (range)^{a} | | | | | | | | | | | | | | | | |
| Median | 5.0 | (5-5) | 4.0 | (2-9) | 2.0 | (1-6) | 2.0 | (2-3) | 2.0 | (1-2) | 1.0 | (1-2) | 2.0 | (2-2) | 2.0 | (1-9) |
| number of | | | | | | | | | | | | | | | | |
| cycles, n | | | | | | | | | | | | | | | | |
| (range) | | | | | | | | | | | | | | | | |
| Median | 15.0 | (15-15) | 16.0 | (13-68) | 14.0 | (8-50) | 18.5 | (17-24) | 11.0 | (10-24) | 8.0 | (7-22) | 36.0 | (36-36) | 16.0 | (7-68) |
| number of | | | | | | | | | | | | | | | | |

doses per
patient, n
(range)

^{a}Treatment duration (weeks) is the (last dosing date-first dosing date + 1) divided by 7

In study 20160132, mean Cₛₛ values during Cycle 1 for the 7-day infusion group (single subjects) were 0.952 and 1.13 ng/mL at doses of 4.5 and 15 μg/day, respectively. Clearance in Cycle 1 was 227 and 580 mL/hr, V_{z} was 1880 and 6070 mL and terminal half-life was estimated to be 5.75 and 7.26 hours at doses of 4.5 and 15 μg/day (Table 5A).

Cₛₛ values (mean±SD) during Cycle 1 for the 28-day infusion group in study 20160132 were 2.02±NR, 3.72±0.61, 9.48±5.43, 26.1±3.99, and 52.8±NR ng/mL at doses of 15, 45, 150, 500, and 1000 μg/day. The inter-subject variability (CV %) in Cₛₛ in Cycle 1 for the 28-day infusion group ranged from 15.3% to 57.3% The mean clearance of AMG 596 in Cycle 1 was estimated to be between 325 and 1450 mL/hr, V_{z} between 2700 to 15900 mL and the mean terminal elimination half-life in Cycle 1 ranged from 5.61 to 8.16 hours (Table 5A).

For the subject enrolled in Study 20180427, mean Cₛₛ in Cycle 3 (corresponds to treatment Cycle 1 of Study 20180427) during week 1 of treatment (1,500 μg/day) was 78.5 ng/mL and 1.32 ng/mL during weeks 2-4 of treatment (15 μg/day) respectively (Table 6). C concentrations during the 15 μg/day treatment period were comparable to Cₛₛ concentrations in subjects receiving 15 μg/day AMG 596 for 7-day on/7-day off cycles and 28-day/14-day off cycles (Table 5A and Table 6). Clearance in Cycle 3 was estimated to be 506 mL/hr, V_{Z} was 5410 mL and the mean terminal elimination half-life was 7.41 hours (Table 6).

Based on an unconfirmed partial response for one patient who received 15/1500 μg/day AMG 596, observed PK data, in vitro assessments of AMG 596 activity and predicted exposures in cerebral spinal fluid (CSF), efficacious exposures of AMG 596 are predicted to be achieved with doses of 1000 to 1500 μg/day.

In summary, serum AMG 596 concentrations increased with increasing dose across all cohorts, exposures increased in an approximate dose proportional manner and a rapid elimination was observed for AMG 596 indicated by the short-observed terminal t_{1/2}.

TABLE 5A
Descriptive Statistics of AMG 596 Pharmacokinetic Parameter Estimates Following
Administration of 4.5 or 15 μg/day AMG 596 cIV 7-day and 15, 45, 150, 500 or 1000
μg/day AMG 596 cIV 28-day Infusion in Subjects with EGFRvIII (Study 20160132)

| | | Summary | t_{1/2,z} | V_{z} | CL | Cₛₛ |
|---|---|---|---|---|---|---|
| Cohort | Cycle | Statistics | (hr) | (mL) | (mL/hr) | (ng/mL) |
| 4.5 μg/day cIV 7-day infusion | 1 | N | 1 | 1 | 1 | 1 |
| | | Mean | 5.75 | 1880 | 227 | 0.952 |
| | | SD | NR | NR | NR | NR |
| | | Min | 5.75 | 1880 | 227 | 0.952 |
| | | Median | 5.75 | 1880 | 227 | 0.952 |
| | | Max | 5.75 | 1880 | 227 | 0.952 |
| | | CV % | NR | NR | NR | NR |
| 15 μg/day cIV 7-day infusion | 1 | N | 1 | 1 | 1 | 1 |
| | | Mean | 7.26 | 6070 | 580 | 1.13 |
| | | SD | NR | NR | NR | NR |
| | | Min | 7.26 | 6070 | 580 | 1.13 |
| | | Median | 7.26 | 6070 | 580 | 1.13 |
| | | Max | 7.26 | 6070 | 580 | 1.13 |
| | | CV % | NR | NR | NR | NR |
| 15 μg/day cIV 28-day infusion | 1 | N | 2 | 2 | 2 | 2 |
| | | Mean | 5.61 | 2700 | 325 | 2.02 |
| | | SD | NR | NR | NR | NR |
| | | Min | 4.37 | 1800 | 285 | 1.73 |
| | | Median | 5.61 | 2700 | 325 | 2.02 |
| | | Max | 6.85 | 3600 | 365 | 2.30 |
| | | CV % | NR | NR | NR | NR |
| 45 μg/day cIV 28-day infusion | 1 | N | 3 | 3 | 3 | 3 |
| | | Mean | 7.09 | 5560 | 544 | 3.72 |
| | | SD | 0.27 | 350 | 14.9 | 0.61 |
| | | Min | 6.87 | 5350 | 530 | 3.30 |
| | | Median | 7.00 | 5370 | 541 | 3.46 |
| | | Max | 7.39 | 5970 | 560 | 4.42 |
| | | CV % | 3.8 | 6.3 | 2.7 | 16.3 |
| 150 μg/day cIV 28-day infusion | 1 | N | 4 | 4 | 4 | 4 |
| | | Mean | 8.16 | 15900 | 1450 | 9.48 |
| | | SD | 0.823 | 18000 | 1760 | 5.43 |
| | | Min | 7.29 | 5550 | 450 | 2.64 |
| | | Median | 8.12 | 7620 | 645 | 10.5 |
| | | Max | 9.10 | 42900 | 4070 | 14.3 |
| | | CV % | 10.1 | 113.3 | 120.8 | 57.3 |
| 500 μg/day cIV 28-day infusion | 1 | N | 3 | 3 | 3 | 3 |
| | | Mean | 5.64 | 6210 | 761 | 26.1 |
| | | SD | 1.10 | 1600 | 93.2 | 3.99 |
| | | Min | 4.37 | 4650 | 681 | 21.7 |
| | | Median | 6.25 | 6140 | 738 | 27.5 |
| | | Max | 6.29 | 7840 | 864 | 29.3 |
| | | CV % | 19.5 | 25.7 | 12.2 | 15.3 |
| 1000 μg/day cIV 28-day infusion | 1 | N | 1 | 1 | 1 | 2 |
| | | Mean | 6.05 | 8160 | 935 | 52.8 |
| | | SD | NR | NR | NR | NR |
| | | Min | 6.05 | 8160 | 935 | 45.9 |
| | | Median | 6.05 | 8160 | 935 | 52.8 |
| | | Max | 6.05 | 8160 | 935 | 59.7 |
| | | CV % | NR | NR | NR | NR |

CL = Apparent clearance after continuous IV infusion; Cₛₛ = Concentration at steady state (24 to 672 hours for cIV 28-day infusion); Cₛₛ = Concentration at steady state (24 to 672 hours for cIV 28-day infusion); NR = Not reported; t_{1/2,z} = Terminal half-life associated with λ_{z}; V_{z} = Apparent volume of distribution after continuous IV infusion
Values are reported to 3 significant figures except for CV %, which was reported to 1 decimal place.

TABLE 6
Descriptive Statistics of AMG 596 Pharmacokinetic Parameter Estimates Following Administration
of 1500/15 μg/day AMG 596 cIV 28-day Infusion in Subjects with EGFRvIII (Study 20180427)

| | | | | | | | Cₛₛ |
|---|---|---|---|---|---|---|---|
| | | | | | | Cₛₛ | Weeks |
| | | Summary | t_{1/2,z} | V_{z} | CL | Week 1 | 2 to 4 |
| Cohort | Cycle | Statistics | (hr) | (mL) | (mL/hr) | (ng/mL) | (ng/mL) |
| 1500/15 μg/day cIV | 3 | N | 1 | 1 | 1 | 1 | 1 |
| 28-day infusion | | Mean | 7.41 | 5410 | 506 | 78.5 | 1.32 |
| | | SD | NR | NR | NR | NR | NR |
| | | Min | 7.41 | 5410 | 506 | 78.5 | 1.32 |
| | | Median | 7.41 | 5410 | 506 | 78.5 | 1.32 |
| | | Max | 7.41 | 5410 | 506 | 78.5 | 1.32 |
| | | CV% | NR | NR | NR | NR | NR |

CL = Apparent clearance after continuous IV infusion; Css = Concentration at steady state (calculated for week 1 and for weeks 2 to 4); NR = Not reported; t_{1/2,z} = Terminal half-life associated with λ_{z}; V_{z} = Apparent volume of distribution after continuous IV infusion
^{a} There were not enough datapoints to calculate t_{1/2,z}, V_{z}, and CL.
Values are reported to 3 significant figures except for CV %, which was reported to 1 decimal place.
Note:
Subject received dose administrations of AMG 596 on Cycle 1 and 2 in the other study (study 20160132). In this study (study 20180427), subject received dose administrations of AMG 596 on Cycle 1 which when counting the two cycles from the previous study (study 20160132) would make this treatment refer to Cycle 3.

Conclusion. Following cIV infusion of AMG 596, steady-state was quickly reached after approximately 24 hours. Serum AMG 596 concentrations increased with increasing dose across all cohorts, exposures increased in an approximate dose proportional manner and AMG 596 exhibited rapid elimination. Efficacious exposures of AMG 596 are predicted to be achieved with doses of 1000 to 1500 μg/day.

5c(2): Updated Observed Clinical PK Results as of 13 Aug. 2020

Updated PK results became available for 28 subjects with recurrent glioblastoma expressing EGFRvIII that received AMG 596 in study 20160132 via continuous infusion.

PK analysis suggests that AMG 596 exposures increased approximately dose proportionally. Observed average steady state concentration (Cₛₛ) in cohorts 1 (4.5 μg/day) to 8 (3000 μg/day) ranged from 0.95 to 178 ng/mL. Steady state was reached within 24 to 48 hours after the start of infusion, which was within the predicted range. A rapid elimination was observed for AMG 596 with an observed preliminary terminal half-life (t_{1/2}) between ˜6 to 8 hours, which is as expected for AMG 596.

The observed steady state concentration of the overdosed patient (1500 mcg per day during week 1 cycle 1) that exhibited an initial 54.6% tumor shrinkage after cycle 1 was 79 ng/mL in serum. Assuming 3.6% or 10% or 20% exposure in CSF, a theoretical concentration of 2.84 ng/mL or 7.9 ng/mL or 15.8 ng/mL could be revealed, all being above the assumed lower threshold of the efficacious concentration range of 1.8 ng/mL. Table 3B summarizes the observed AMG 596 PK. From Cohort 6 (1000 μg/day) and onwards, exposure levels are within or above the range of projected minimum efficacious exposures. Table 4B shows that nineteen patients received AMG 596 for a median duration of 9 weeks (range 4-52 weeks).

TABLE 3B
Observed average steady state concentration (C_{SS})

| Cohort | Dose (ug/day) | Observed C_{SS} (ng/mL) | N |
|---|---|---|---|
| 1 | 4.5 | 0.95 | 1 |
| 2a/2b | 15 | 2.0 | 3 |
| 3 | 45 | 3.7 | 3 |
| 4 | 150 | 9.5 | 4 |
| 5 | 500 | 26 | 3 |
| 6 | 1000 | 67 | 4 |
| Overdose | 1500 | 79 | 1 |
| 7 | 1500 | 112 | 4 |
| 8 | 3000 | 178 | 5 |

TABLE 4B
AMG 596 Exposure

| | 4.5 | 15 | 45 | 150 | 500 | 1000 | 1500 | 3000 | 6000 | 1500/15 | |
|---|---|---|---|---|---|---|---|---|---|---|---|
| | μg/d | μg/d | μg/d | μg/d | μg/d | μg/d | μg/d | μg/d | μg/d | μg | All |
| | (N = 1) | (N = 3) | (N = 3) | (N = 4) | (N = 3) | (N = 4) | (N = 4) | (N = 4) | (N = 1) | (N = 1) | (N = 29) |
| Median | 9.0 | 10.1 | 11.1 | 11.4 | 8.1 | 10.4 | 10.2 | 13.6 | 8.1 | 10.1 | 9.0 |
| duration, wk | (9-9) | (9-98) | (4-40) | (10-16) | (4-9) | (4-52) | (4-35) | (2-25) | (8-8) | (10-10) | (4-52) |
| (range)³ | | | | | | | | | | | |
| Median | 5.0 | 4.0 | 2.0 | 2.0 | 2.0 | 2.0 | 2.0 | 3.0 | 2.0 | 2.0 | 2.0 |
| number of | (5-5) | (2-16) | (1-7) | (2-3) | (1-2) | (1-8) | (1-6) | (1-5) | (2-2) | (2-2) | (1-16) |
| cycles, n | | | | | | | | | | | |
| (range) | | | | | | | | | | | |
| Median | 15.0 | 16.0 | 14.0 | 18.5 | 11.0 | 20.0 | 24 | 22 | 13 | 36.0 | 18.0 |
| number of | (15-15) | (13-126) | (8-59) | (17-24) | (10-28) | (8-58) | (17-50) | (1-35) | (13-13) | (36-36) | (1-126) |
| days on | | | | | | | | | | | |
| infusion, | | | | | | | | | | | |
| days (range) | | | | | | | | | | | |

^{a}Treatment duration (weeks) is the (last dosing date-first dosing date + 1) divided by 7

Cₛₛ values (mean±SD) during Cycle 1 for the 28-day infusion group in study 20160132 were 2.02±NR, 3.72±0.61, 9.48±5.43, 26.1±3.99, 67.8±17.4, 112±25.6 and 178±55.1 ng/mL at doses of 15, 45, 150, 500, 1000, 1500 and 3000 μg/day. The inter-subject variability (CV %) in Cₛₛ in Cycle 1 for the 28-day infusion group ranged from 15% to 57%. The mean clearance of AMG 596 in Cycle 1 was estimated to be between 227 and 1450 mL/hr, V_{z} between 1880 to 15900 mL and the mean terminal elimination half-life in Cycle 1 ranged from 5.61 to 8.16 hours (Table 5B).

Based on these studies, efficacious exposures of AMG 596 are predicted to be achieved with doses of 1000 μg/day or higher (preferably 1500 μg/day or higher, such as from 1500 to 3000 μg/day). Although efficacious exposure can be achieved with doses as low as 15 μg/day (initial tumor shrinkage indeed overserved), better results were observed after dose escalation to 1000 μg/day or higher.

In addition, preliminary AMG 596 CSF PK results became available for 3 subjects. Based on limited available data, AMG 596 serum to CSF penetration is variable between patients (up to ˜6-fold difference between quantifiable samples) and between ˜ 0.3% and 1.7%. Out of 6 available CSF samples, only 2 samples taken during AMG 596 infusion had quantifiable AMG 596 levels which is in line with the relatively short observed half-life of AMG 596 in serum.

TABLE 5B
Descriptive Statistics of AMG 596 Pharmacokinetic Parameter Estimates Following Administration
of 4.5 or 15 μg/day AMG 596 cIV 7-day and 15, 45, 150, 500, 1000, 1500 or 3000 μg/day AMG
596 cIV 28-day Infusion in Subjects with EGFRvIII (Study 20160132)

| | | Summary | t_{1/2,z} | V_{z} | CL | Cₛₛ |
|---|---|---|---|---|---|---|
| Cohort | Cycle | Statistics | (hr) | (mL) | (mL/hr) | (ng/mL) |
| 4.5 μg/day cIV 7-day infusion | 1 | N | 1 | 1 | 1 | 1 |
| | | Mean | 5.75 | 1880 | 227 | 0.952 |
| | | SD | NR | NR | NR | NR |
| | | Min | 5.75 | 1880 | 227 | 0.952 |
| | | Median | 5.75 | 1880 | 227 | 0.952 |
| | | Max | 5.75 | 1880 | 227 | 0.952 |
| | | CV % | NR | NR | NR | NR |
| 15 μg/day cIV 7-day infusion | 1 | N | 1 | 1 | 1 | 1 |
| | | Mean | 7.26 | 6070 | 580 | 1.13 |
| | | SD | NR | NR | NR | NR |
| | | Min | 7.26 | 6070 | 580 | 1.13 |
| | | Median | 7.26 | 6070 | 580 | 1.13 |
| | | Max | 7.26 | 6070 | 580 | 1.13 |
| | | CV % | NR | NR | NR | NR |
| 15 μg/day cIV 28-day infusion | 1 | N | 2 | 2 | 2 | 2 |
| | | Mean | 5.61 | 2700 | 325 | 2.02 |
| | | SD | NR | NR | NR | NR |
| | | Min | 4.37 | 1800 | 285 | 1.73 |
| | | Median | 5.61 | 2700 | 325 | 2.02 |
| | | Max | 6.85 | 3600 | 365 | 2.30 |
| | | CV % | NR | NR | NR | NR |
| 45 μg/day cIV 28-day infusion | 1 | N | 3 | 3 | 3 | 3 |
| | | Mean | 7.09 | 5560 | 544 | 3.72 |
| | | SD | 0.27 | 350 | 14.9 | 0.61 |
| | | Min | 6.87 | 5350 | 530 | 3.30 |
| | | Median | 7.00 | 5370 | 541 | 3.46 |
| | | Max | 7.39 | 5970 | 560 | 4.42 |
| | | CV % | 4 | 6 | 3 | 16 |
| 150 μg/day cIV 28-day infusion | 1 | N | 4 | 4 | 4 | 4 |
| | | Mean | 8.16 | 15900 | 1450 | 9.48 |
| | | SD | 0.823 | 18000 | 1760 | 5.43 |
| | | Min | 7.29 | 5550 | 450 | 2.64 |
| | | Median | 8.12 | 7620 | 645 | 10.5 |
| | | Max | 9.10 | 42900 | 4070 | 14.3 |
| | | CV % | 10 | 113 | 121 | 57 |
| 500 μg/day cIV 28-day infusion | 1 | N | 3 | 3 | 3 | 3 |
| | | Mean | 5.64 | 6210 | 761 | 26.1 |
| | | SD | 1.10 | 1600 | 93.2 | 3.99 |
| | | Min | 4.37 | 4650 | 681 | 21.7 |
| | | Median | 6.25 | 6140 | 738 | 27.5 |
| | | Max | 6.29 | 7840 | 864 | 29.3 |
| | | CV % | 19 | 26 | 12 | 15 |
| 1000 μg/day cIV 28-day infusion | 1 | N | 4 | 4 | 4 | 4 |
| | | Mean | 6.24 | 6010 | 663 | 67.0 |
| | | SD | 1.63 | 2180 | 177 | 17.4 |
| | | Min | 4.01 | 3090 | 525 | 46.4 |
| | | Median | 6.73 | 6530 | 612 | 68.3 |
| | | Max | 7.49 | 7870 | 902 | 85.0 |
| | | CV % | 26 | 36 | 27 | 26 |
| 1500 μg/day cIV 28-day infusion | 1 | N | 4 | 4 | 4 | 4 |
| | | Mean | 6.61 | 5810 | 623 | 112 |
| | | SD | 1.37 | 1270 | 166 | 25.6 |
| | | Min | 5.32 | 4460 | 478 | 75.2 |
| | | Median | 6.31 | 5650 | 576 | 119 |
| | | Max | 8.52 | 7500 | 862 | 134 |
| | | CV % | 21 | 22 | 27 | 23 |
| 3000 μg/day cIV 28-day infusion | 1 | N | 4 | 4 | 4 | 5 |
| | | Mean | 6.43 | 7590 | 835 | 178 |
| | | SD | 0.621 | 2750 | 366 | 55.1 |
| | | Min | 5.82 | 5230 | 552 | 92.1 |
| | | Median | 6.34 | 6800 | 708 | 182 |
| | | Max | 7.24 | 11500 | 1370 | 243 |
| | | CV % | 10 | 36 | 44 | 31 |

Conclusion. Efficacious exposures of AMG 596 are predicted to be achieved with doses of 1000 μg/day or higher (preferably 1500 μg/day or higher). While it is believed that 1500 to 3000 μg/day is preferable, dosing higher than 3000 μg/day is expected to efficacious as well, as observed in the 6000 μg/day cohort. Therefore, in some circumstances, it may be desirable to administer 3000 μg/day to 12000 μg/day.

### Example 6: Efficacy Results

6a. Efficacy Results from Interim Analysis

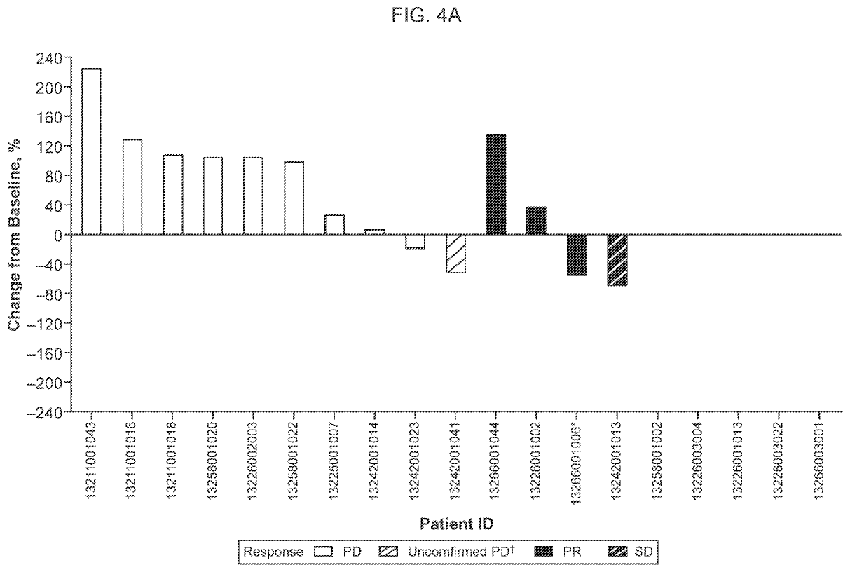

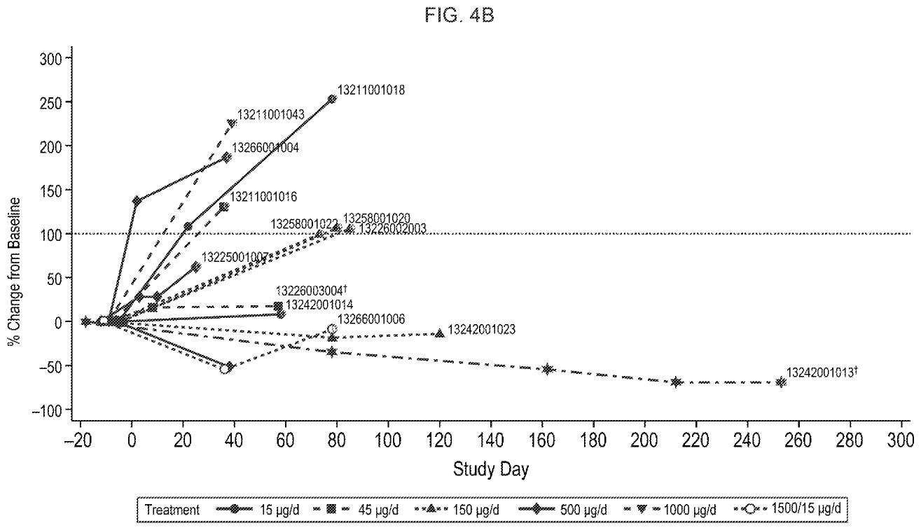

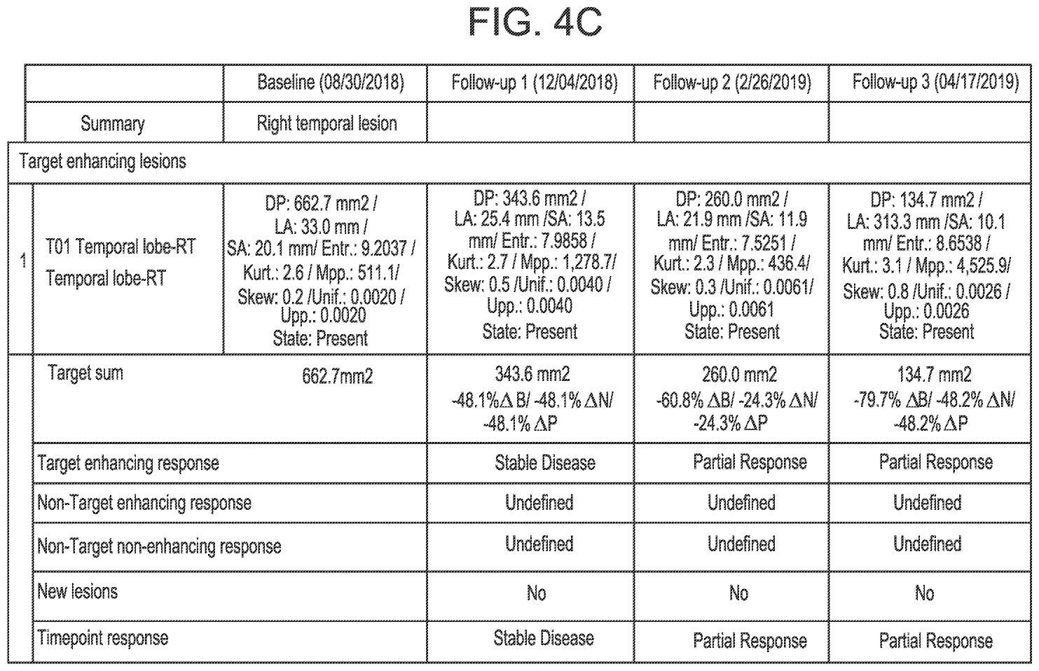

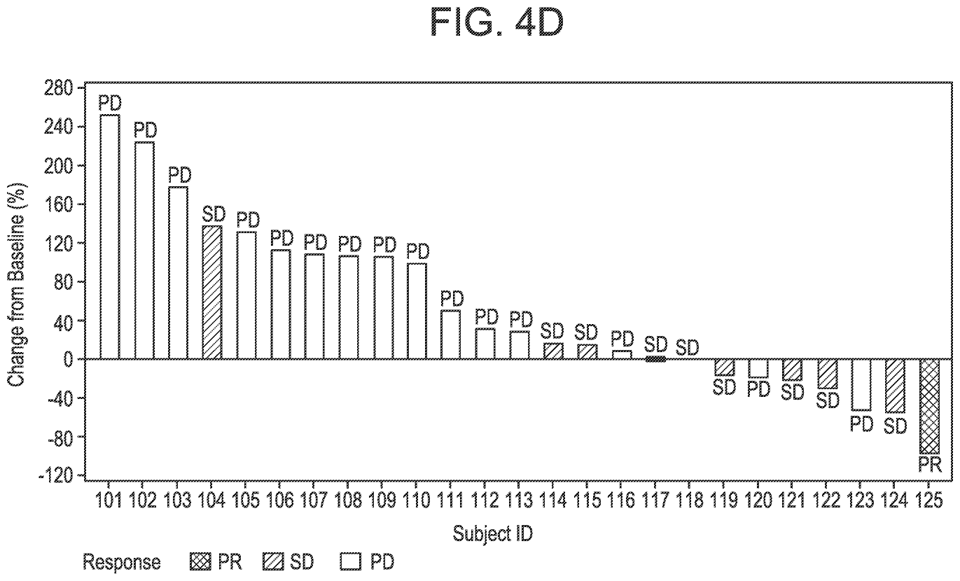

As of the data cutoff date (1 Jul. 2019), safety and efficacy data were available for 15 subjects. 1 subject (42001013) treated at the dose of 15 μg had a partial response (PR), which was ongoing at the data cutoff date, 2 subjects had stable disease (1 subject at 45 μg and 1 subject who received 1500 μg AMG 596); 5 subjects had progressive disease. FIGS. 4A-4C show the change in tumor size.

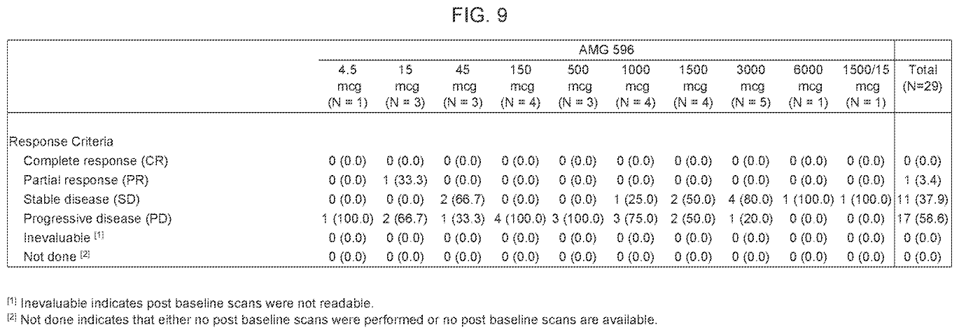

As of the cutoff date 13 Aug. 2020, safety and efficacy data were available for 29 subjects who were enrolled and received at least one dose with AMG 596. In this Safety Analysis Set (n=29), clinical benefit with best response of PR or SD was observed in 12 (41.3%) subjects: 1 (3.4%) PR and 11 (37.9%) SD, and PD was observed in 17 (58.6%) subjects (FIG. 9). The subject with PR was treated at a start dose of 15 μg and is ongoing in partial remission at the cut-off date 8 Sep. 2020 with 98.6% tumor shrinkage seen after dose escalation to 1500 μg. Eleven subjects presented with stable disease at first imaging evaluation after start of treatment (FIG. 9) according to investigators' evaluation but one of these subjects did not complete a first treatment cycle and was therefore removed from further analyses. Six out of 10 subjects had durable stable disease with a time to progression of >90 days. One additional subject has been ongoing in stable disease with a follow-up of 77 days at time of the cut-off date 8 Sep. 2020. All subjects with durable stable disease had a dose escalation to 1000 μg or 1500 μg or treatment started at a dose of 1500 μg or higher. The two subjects with dose escalation during treatment had not received prophylactic dexamethasone at treatment start with a lower dose (Table 10).

TABLE 10
Progression free survival versus dose for all subjects who
completed one AMG 596 cycle and had SD or PR in tumor

| | Number of | Maximum | Time to | | | |
|---|---|---|---|---|---|---|
| | treatment | Tumor | Progression | Best | Start Dose | Highest Dose |
| Subject | cycles | shrinkage | (days) | Response | (μg per day) | (μg per day) |
| 26001002 | 2 | (+15.9%) | 57 | SD | 45* | 45 |
| 26003004 | 7 | n.a. | 161 | SD | 45* | 1000 |
| 66001006 | 2 | −54.60% | 78 | SD | 1500* | 1500 |
| | | | | | (15) | |
| 42001013 | 16+ | −98.60% | 628+ | PR | 15* | 1500 |
| 26003022 | 8 | n.a. | 254 | SD | 1000 | 3000 |
| 26001007 | 2 | (+14.6%) | 78 | SD | 1500 | 1500 |
| 26003027 | 7+ | n.a. | 160 | SD | 1500 | 3000 |
| 25001043 | 3+ | 0 | 148+ | SD | 3000 | 3000 |
| 42001067 | 3 | −29.50% | 108 | SD | 3000 | 3000 |
| 42001072 | 5+ | −21.24% | 104 | SD | 3000 | 3000 |
| 42001074 | 3+ | −4.10% | 77+ | SD | 6000 | 6000 |

*No prophylactic dexamethasone at start of treatment
+ongoing at cut-off Sep. 8, 2020

Treatment with higher dose and longer treatment duration resulted in better disease control independent of EGFRvIII expression level. The median (range) number of cycles received for all 29 subjects was 2.0 (1 to 16). The median (range) treatment duration was 10.1 (2 to 98) weeks. Subjects with a best response of PR or SD received at least 2 treatment cycles and 7 out of 11 subjects received 3 or more treatment cycles. As of the cut-off date 8 Sep. 2020, median time to progression was numerically longer for subjects with SD or PR versus subjects with PD (108 days, range 57-628 versus 58 days, range 3-85), and for subjects with a highest dose of 1000 μg or higher versus subjects with highest dose of 500 μg or lower (78 days, range 30-628, versus 59 days, range 3-85). No numerical difference was seen in median time to progression and EGFRvIII expression presented by H-score (72.5 days, range 3-254, for H-score 100 to 285 and 73 days, range 22-628, for H-score 1 to 75). See Table 11.

TABLE 11

| Parameters | Median Time to progression (days) |
|---|---|
| All Subjects | 70,5 (range 3-628) |
| Best Response PD | 58 (range 3-85) |
| Best Response PR/SD | 108 (range 57-628) |
| Highest Dose | 59 (range 3-85) |
| (4.5-500 ug/d) | |
| Highest Dose | 78 (range 30-628) |
| (1000-3000 ug/d) | |
| EGFRvlll Expression | 72,5 (range 3-254) |
| (H-Score 100-285) | |
| EGFRvlll Expression | 73 (range 22-628) |
| (H-Score 1-75) | |

6b. Case Report on Observation of a Confirmed PR

The subject with PR is a 44 y/o female with initial diagnosis of glioblastoma in October 2017. The subject underwent tumor resection on 26 Oct. 2017 with residual disease remaining. The tumor was found to be EGFRvIII positive (70% of cells with positive staining). Between 28 Nov. 2017 and 9 Jan. 2018, the subject received external beam radiation followed by temozolomide maintenance therapy until 9 Jun. 2018. On 18 Jul. 2018 tumor evaluation by imaging revealed tumor progression. The subject was screened for this study and started treatment on 18 Sep. 2018 at an AMG 596 dose of 15 ug per day. Tumor evaluations were done after every 2nd cycle revealing tumor shrinkage starting after cycle 2. Maximum shrinkage was observed after cycle 6 with 79.7% tumor shrinkage versus baseline confirmed by external read evaluation. Although the subject was treated at a dose of 15 ug per day that is assumed to be below the potentially efficacious dose range, PK measurements showed higher than expected exposures in cycles 4 (Cmax of 9.51 ng/mL) and 6 (Cmax 6.7 ng/mL) and antitumor efficacy is hinting to a 20% or higher CSF exposure. No other antitumor treatment was given to the subject. Also, recovery from pseudoprogression after radiotherapy can be ruled out due to the long-time interval between radiation and start of AMG 596 therapy. The tumor shrinkage is clearly seen as effect of AMG 596 therapy (FIG. 4C).

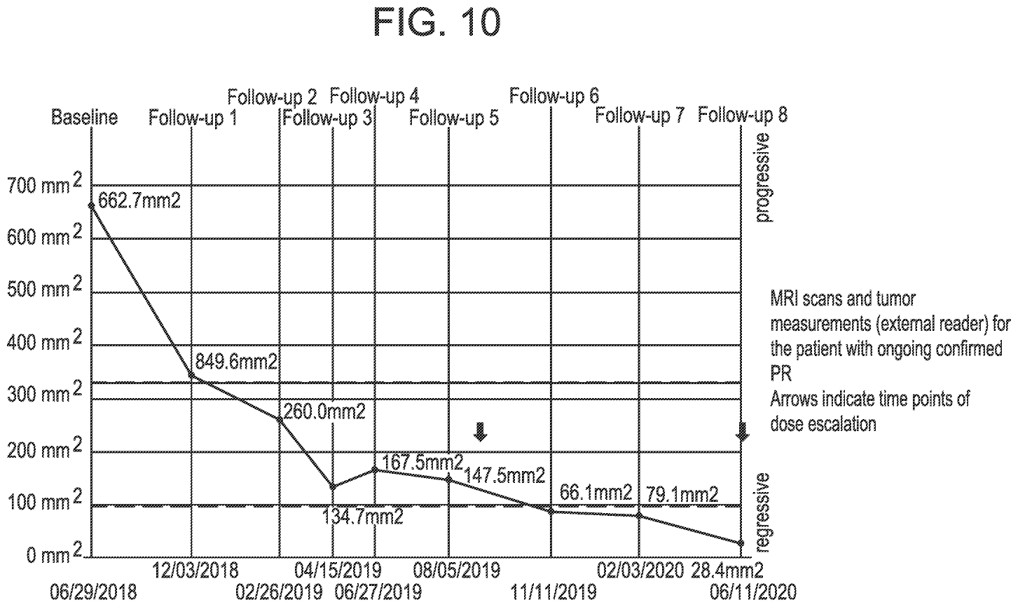

Subsequently, the PR was maintained and 2 dose escalation steps after treatment breaks of 4 weeks were performed until the cut-off date 8 Sep. 2020. A dose escalation to 500 μg with treatment cycle 9 resulted in 86.7% tumor shrinkage (FIG. 10) and further dose escalation to 1500 μg in cycle 16 resulted in 98.9% tumor shrinkage, both observed in the following tumor evaluations after dose escalation.

### Example 7: Safety Results of Interim Analysis and Associated with AMG 596 Pharmacodynamic Activity

7a. Safety Up to a Dose of 1500 μg/Day

As described earlier, EGFRvIII is considered a bona-fide tumor-specific antigen found exclusively on tumor cells and the EGFRvIII mutation can rarely be found on normal tissue cells in human. In consequence, considering that the mechanism of action of a BiTE® molecule requires the availability of all three components, BiTE® molecule plus T cell plus target expressed on cells, any clinical observations attributed to AMG 596 is indicating AMG 596 has engaged T cells for killing of EGFRvIII positive tumor cells.

Although no dose limiting toxicities (DLTs) were observed in any of the 15 subjects who have received AMG 596 doses up to 1500 ug per day, treatment-related adverse events have been observed in 13/15 subjects covered in the interim analysis. The most frequently reported adverse events (occurring in more than 2 subjects) were headache (n=11; 73.3%), nausea and vomiting (n=6 for each; 40.0%), seizure and fatigue (n=5 for each; 33.3%), diarrhea and asthenia (n=4 for each; 26.7%), and aphasia, somnolence, gait disturbance, pyrexia, and lymphopenia (n=3 for each; 20.0%). Eight subjects (53.3%) had adverse events that were grade>3 in severity (depressed level of consciousness and headache for 2 subjects each, and aphasia, hypersomnia, pyramidal tract syndrome, seizure, syncope, glioblastoma, glioblastoma multiforme, device related thrombosis, lung infection, pneumonia, shunt malfunction, blood creatine phosphokinase increased, and hypertension for 1 subject each). Neurologic adverse events were seen in subjects treated at AMG 596 doses of 15 ug per day or higher specifically.

TABLE 7
Treatment-Related AEs by System Organ Class

| | 4.5 μg | 15 μg | 45 μg | 150 μg | 500 μg | 1500/15 μg | All |
|---|---|---|---|---|---|---|---|
| | (N = 1) | (N = 3) | (N = 3) | (N = 4) | (N = 2) | (N = 1) | (N = 14) |
| Treatment-related | 1 (100.0) | 3 (100.0) | 3 (100.0) | 3 (75.0) | 2 (100.0) | 1 (100.0) | 13 (92.9) |
| TEAE, n (%) | | | | | | | |
| General disorders | 1 (100.0) | 3 (100.0) | 2 (66.7) | 1 (25.0) | 1 (50.0) | 1 (100.0) | 9 (64.3) |
| and administration | | | | | | | |
| site conditions | | | | | | | |
| Nervous system | 0 | 2 (66.7) | 2 (66.7) | 2 (50.0) | 2 (100.0) | 1 (100.0) | 9 (64.3) |
| disorders | | | | | | | |
| Gastrointestinal | 1 (100.0) | 1 (33.3) | 1 (33.3) | 1 (25.0) | 1 (50.0) | 1 (100.0) | 6 (42.9) |
| disorders | | | | | | | |
| Lab results | 1 (100.0) | 1 (33.3) | 1 (33.3) | 0 | 0 | 1 (100.0) | 4 (28.6) |
| Blood and | 1 (100.0) | 1 (33.3) | 1 (33.3) | 0 | 0 | 0 | 3 (21.4) |
| lymphatic system | | | | | | | |
| disorders | | | | | | | |
| Immune system | 0 | 0 | 0 | 1 (25.0) | 0 | 1 (100.0) | 2 (14.3) |
| disorders | | | | | | | |
| Injury, poisoning, | 1 (100.0) | 0 | 0 | 0 | 0 | 1 (100.0) | 2 (14.3) |
| and procedural | | | | | | | |
| complications | | | | | | | |
| Skin and | 1 (100.0) | 0 | 0 | 1 (25.0) | 0 | 0 | 2 (14.3) |
| subcutaneous | | | | | | | |
| tissue disorders | | | | | | | |
| (alopecia) | | | | | | | |
| Eye disorders | 0 | 0 | 0 | 0 | 0 | 1 (100.0) | 1 (7.1) |
| (visual impairment) | | | | | | | |
| Metabolism and | 1 (100.0) | 0 | 0 | 0 | 0 | 0 | 1 (7.1) |
| nutrition disorders | | | | | | | |
| Musculoskeletal | 0 | 1 (33.3) | 0 | 0 | 0 | 0 | 1 (7.1) |
| and connective | | | | | | | |
| tissue disorders | | | | | | | |
| Psychiatric | 0 | 0 | 0 | 1 (25.0) | 0 | 0 | 1 (7.1) |
| disorders | | | | | | | |
| (bradyphrenia) | | | | | | | |
| Renal/urinary | 0 | 0 | 1 (33.3) | 0 | 0 | 0 | 1 (7.1) |
| disorders | | | | | | | |
| (hematuria) | | | | | | | |
| Respiratory, | 0 | 1 (33.3) | 0 | 0 | 0 | 0 | 1 (7.1) |
| thoracic, and | | | | | | | |
| mediastinal | | | | | | | |
| disorders (hiccups) | | | | | | | |

TABLE 8
Patient Disposition: five patients (25%) ongoing on AMG
596 for more than 4 weeks (range: 4-52 weeks)

| | | | | | | | 1500/ | |
|---|---|---|---|---|---|---|---|---|
| | 4.5 μg/d | 15 μg/d | 45 μg/d | 150 μg/d | 500 μg/d | 1000 μg/d | 15 μg/d | All |
| Patients, n (%) | (n = 1) | (n = 3) | (n = 3) | (n = 4) | (n = 4) | (n = 4) | (n = 1)* | (N = 19) |
| Enrolled | 1 | 3 | 3 | 4 | 4 | 4 | 1 | 20 |
| | (100.0) | (100.0) | (100.0) | (100.0) | (100.0) | (100.0) | (100.0) | (100.0) |
| Patients who | 1 | 3 | 3 | 4 | 3 | 4 | 1 | 19 |
| received | (100.0) | (100.0) | (100.0) | (100.0) | (75.0) | (100.0) | (100.0) | (95.0) |
| AMG 596 | | | | | | | | |
| Patients | — | 1 | 1 | — | — | 3 | — | 5 |
| continuing | | (33.3) | (33.3) | | | (75.0) | | (25.0) |
| AMG 596 | | | | | | | | |
| Patients who | 1 | 2 | 2 | 4 | 3 | 1 | 1 | 14 |
| discontinued | (100.0) | (66.7) | (66.7) | (100.0) | (75.0) | (25.0) | (100.0) | (70.0) |
| investigational | | | | | | | | |
| product | | | | | | | | |
| Adverse | — | — | — | — | 1 | — | — | 1 |
| event | | | | | (25.0) | | | (5.0) |
| Disease | 1 | 2 | 2 | 4 | 2 | 1 | 1 | 13 |
| progression | (100.0) | (66.7) | (66.7) | (100.0) | (50.0) | (25.0) | (100.0) | (65.0) |
| Patients | — | 1 | 1 | 1 | — | 4 | — | 7 |
| continuing | | (33.3) | (33.3) | (25.0) | | (100.0) | | (35.0) |
| study | | | | | | | | |
| Patients who | 1 | 2 | 2 | 3 | 4 | — | 1 | 13 |
| discontinued | (100.0) | (66.7) | (66.7) | (75.0) | (100.0) | | (100.0) | (65.0) |
| study | | | | | | | | |
| Withdrawal | — | — | — | — | 1 | — | 1 | 2 |
| of consent | | | | | (25.0) | | (100.0) | (10.0) |
| from study | | | | | | | | |
| Decision by | — | — | — | — | — | — | — | — |
| sponsor | | | | | | | | |
| Lost to | — | — | — | — | — | — | — | — |
| follow-up | | | | | | | | |
| Death | — | 1 | 1 | 1 | 1 | — | — | 4 |
| | | (33.3) | (33.3) | (25.0) | (25.0) | | | (20.0) |
| Start of any | 1 | 1 | 1 | 2 | 2 | — | — | 7 |
| other | (100.0) | (33.3) | (33.3) | (50.0) | (50.0) | | | (35.0) |
| antitumor | | | | | | | | |
| therapy | | | | | | | | |

*Data are summarized by planned treatment. Pt 13266001006 (enrolled in Cohort 2b) received an overdose (1500 μg/day) and has entered expanded access study based on FDA recommendation to receive 1500 μg/day during week 1, followed by 15 μg/day during weeks 2-4, followed by a 2-week break. As summarized in Table 8, 19 (95.0%) patients received AMG 596, of which 5 patients (25.0%) continued on AMG 596. 13 (65.0%) patients discontinued AMG 596 due to disease progression and 1 (5.0%) discontinued due to AE.

TABLE 9
Disease control was reported in five patients treated with
AMG 596 (PR in one patient, SD in four patients)
Best overall response in patients with sufficient
follow-up to assess response to treatment

| | | | AMG 596 | | | | | | |
|---|---|---|---|---|---|---|---|---|---|
| | | | dose | | | | | Response | Tumor |
| | n (%) | | received | | Treatment duration | | | duration | shrinkage |
| Partial | 1 | (5.6%) | 15 | μg/day | 52 | weeks | 9 cycles | 92 days | 70% |
| response | | | | | | | | | |
| (PR) | | | | | | | | | |
| Stable | 4 | (22.2%) | 45 | μg/day | 11 | weeks | 2 cycles | n/a | n/a |
| disease | | | 45 | μg/day | 34 | weeks | 6 cycles | | |

| | | | | | |
|---|---|---|---|---|---|
| (SD) | 500 | μg/day | 8 | weeks | 2 cycles |
| | 1500 | μg/day | 10 | weeks | 2 cycles |

Unconfirmed PR that converted to SD was reported in one patient who received 1500/15 μg/day AMG 596
PD was present in nine patients at first RANO assessment scan
RANO response was not available at time of analysis in four patients
The clinical observation of treatment-related adverse events at various AMG 596 doses supports the pharmacodynamic activity of AMG 596.

On 30 Apr. 2019, a new safety signal was identified in patients administered AMG 596 in this Study. Of the 15 subjects who have been exposed to AMG 596, 3 subjects experienced events of depressed level of consciousness with the following preferred terms: (i) depressed level of consciousness (n=2 serious events), Cohort 4 (150 μg/day); (ii) depressed level of consciousness (n=3 events: 2 serious, 1 nonserious), Cohort 3 (45 μg/day); (iii) syncope (n=1 serious event), Cohort 2 (15 μg/day); (iv) somnolence (n=2 events: 1 serious, 1 nonserious), Cohort 2 (15 μg/day).

Patients with glioblastoma multiforme (GBM) are at an increased risk for events of decreased level of consciousness, usually due to cerebral edema. AMG 596 administration in GBM subjects may cause a dose dependent increase of peritumoral edema around the GBM leading to the development or worsening of depressed level of consciousness.

The prophylactic use of corticosteroids during treatment with AMG 596 is now mandated and has been initiated with Cohort 6 and AMG 596 treatment at 1000 ug per day.

7b. Safety Update with AMG 596 Treatment Up to 6000 μg

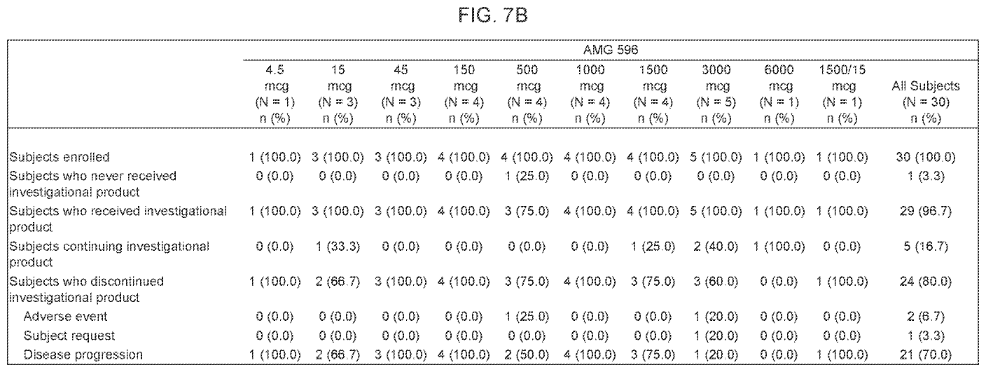

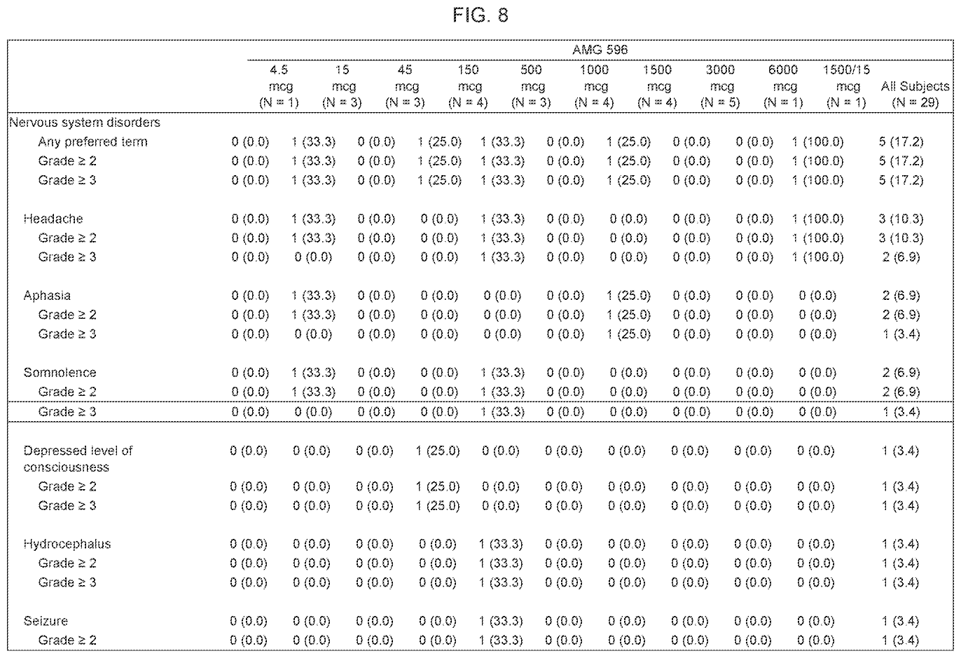

Safety was maintained with prophylactic use of corticosteroids and no new safety signal up to a dose of 6000 μg per day was observed. Of 29 subjects who received at least 1 dose of AMG 596, 28 subjects (96.6%) had at least 1 treatment emergent adverse event (hereafter referred to as adverse events). Twenty-five subjects (86.2%) had at least 1 adverse event that was considered by the investigator as related to AMG 596 treatment. The most frequently reported adverse events (occurring in >2 subjects) were headache (n=20, 69.0%); fatigue (n=12, 41.4%); aphasia, nausea, and seizure (n=9, 31.0% each); pyrexia and vomiting (n=7, 24.1% each); asthenia and diarrhea (n=6, 20.7% each); aspartate aminotransferase increased, dizziness, and gait disturbance (n=5, 17.2% each); alanine aminotransferase increased, anemia, C reactive protein increased, constipation, hemiparesis, lymphopenia, rash, and somnolence (n=4, 13.8% each); and abdominal pain, alopecia, decreased appetite, device related infection, dyspepsia, glioblastoma, hypokalemia, leukopenia, syncope, and white blood cell count decreased (n=3, 10.3% each). Depressed level of consciousness was reported for 2 subjects up to the data cutoff date of 13 Aug. 2020. Seventeen subjects (58.6%) had a serious adverse event(s) after receiving AMG 596. Of these, 12 subjects (41.4%) had events that were grade ≥3 in severity; these events (reported in ≥2 subjects) included glioblastoma (n=3, 10.3%) and depressed level of consciousness, device-related infection, headache, and syncope (n=2, 6.9% each). Five subjects (17.2%) had fatal adverse events caused by progression of underlying disease (4 subjects had glioblastoma and 1 subject had general physical health deterioration due to disease progression). Three (10.3%) subjects had adverse events that led to discontinuation of AMG 596 treatment (anxiety, cerebrovascular accident, cognitive disorder, dizziness, fatigue, psychiatric disorders, and somnolence). No adverse events led to a dose reduction of AMG 596. Ten (34.5%) subjects had adverse events that led to treatment interruption; these events (reported in ≥2 subjects) included aphasia reported for 3 subjects; and depressed level of consciousness, device-related infection, and pyrexia reported for 2 subjects each. No DLTs were observed in any of the 29 subjects who received AMG 596 6000 μg 6000 μg once daily. See, FIG. 8.

### Example 8: Case Report on Observations with AMG 596 Treatment at 1500 ug Per Day

Subject 13266001006 enrolled to this Study had glioblastoma with recurrent disease after standard of care treatment, with a poor prognosis. The subject was enrolled to Cohort 2b (15 ug/day cIV 28 days on followed by 14 days off). During cycle 1, days 1-7, the subject received an overdose (1500 ug/day) due to a mixing error by the pharmacy. The subject had an unscheduled tumor evaluation following infusion in cycle 1. The MRI showed a 58% decrease in tumor burden. The result was confirmed by an external read evaluation according to the study procedures. The Principal Investigator (PI) attributed the observed response to the high dose of AMG 596 the subject received during the first week of Cycle 1. The observed drug exposure in the subject's serum during the treatment at 1,500 μg/day matched the predicted potential efficacious exposure level. Despite a short (2 hours) episode of Grade 3 headache that responded to pain medication and dexamethasone, the subject tolerated the 1,500 μg/day dose and adverse events resolved with ongoing high dose treatment.

Medical History. Subject 13266001006 is a 49 Y/o female with initial diagnosis of glioblastoma in November 2017. After surgery in November 2017, radiotherapy between December 2017 and February 2018 and chemotherapy (temozolomide until May 2018 and CCNU in June 2018) the subject presented with disease progression on 17 Aug. 2018 and was screened for the study.

AMG 596 Treatment and Results Details. AMG 596 therapy was initiated on 10 Sep. 2018. The initial dose during week one was 1500 ug per day due to a dosing error. The subject continued treatment at the regularly planned dose of 15 mcg starting week 2 in cycle 1. Tumor evaluation at end of cycle 1 revealed 57.9% shrinkage in tumor load versus baseline (unconfirmed PR). The treatment at 15 ug per day in cycle 2 led to tumor growth and the response could not be maintained. The PK evaluation showed a steady state exposure of 79 ng/mL during week 1 that decreased to below 1 ng/mL in cycle 2 with treatment at 15 ug per day.

### Example 9: EGFRvIII Expression

Since AMG 596 specifically targets EGFRvIII, and is expected to have clinical effect in tumors expressing EGFRvIII, prospective selection of patients with EGFRvIII positive tumors is desirable for clinical development. An immunohistochemical (IHC) assay with an exemplary EGFRvIII antibody is used for patient selection for the AMG 596 phase I study in recurrent GBM.

Methods. Described herein is an EGFRvIII IHC assay containing optimized reagents and protocols to complete an IHC staining procedure of FFPE specimens (5-um-thick sections). Following incubation with the primary monoclonal antibody to EGFRvIII, specimens were incubated with a linker antibody specific to the host species of the primary antibody, and then were incubated with ready-to-use visualization reagent comprising secondary antibody molecules and horseradish peroxidase. The enzymatic conversion of the subsequently added chromogen resulted in precipitation of a visible reaction product at the site of antigen. The specimen was counter-stained and cover slipped. Results were interpreted using a light microscope.

A semi-quantitative H-score method assigns an IHC H-score to each patient on a continuous scale of 0-300, based on the percentage of tumor cells at different staining intensities visualized at different magnifications. Membrane staining was scored according to four categories: 0 for ‘no staining’, 1+ for ‘light staining visible only at high magnification’, 2+ for ‘intermediate staining’ and 3+ for ‘dark staining of linear membrane. The percentage of cells at different staining intensities was determined by visual assessment, with the score calculated using the formula: 1×(% of 1+ cells)+2×(% of 2+ cells)+3×(% of 3 cells). The IHC scoring assessment was performed by a trained pathologist in a commercial lab.

Patients underwent radiologic assessment using MRI; subsequent tumor evaluations by MRI occurred at later time point. Tumor response was assessed per RANO criteria. Patients with an objective response (defined as a complete or partial response) received a second MRI scan for confirmation after the criteria for response were met.

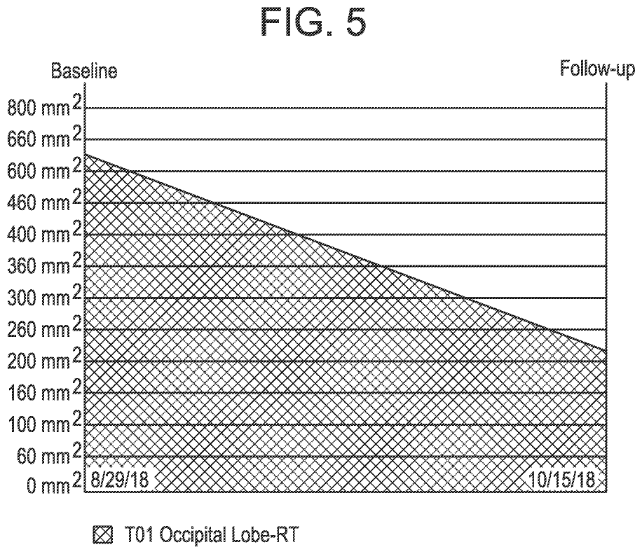

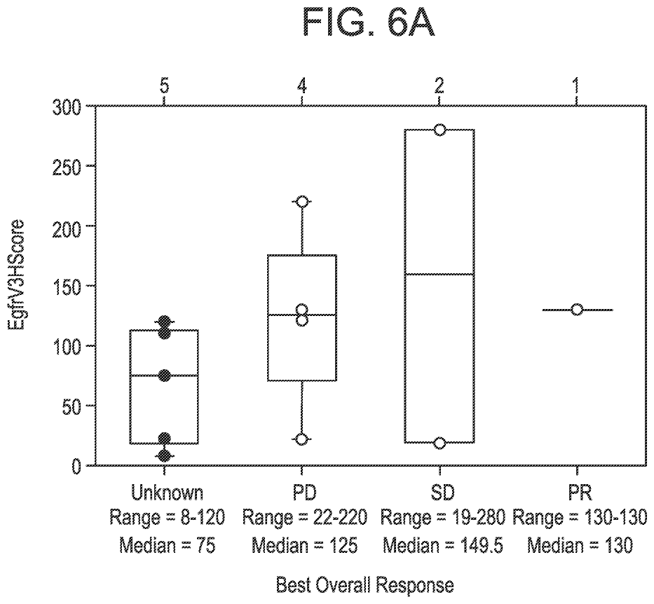

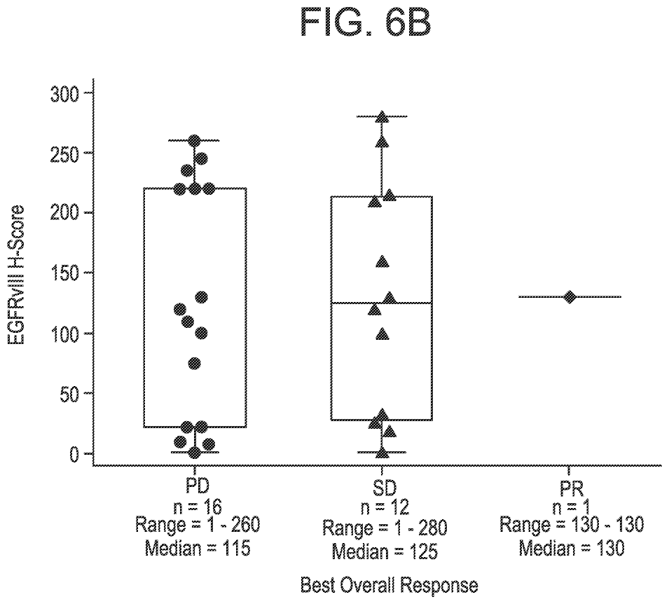

Results. FIG. 6A summarizes patient's EGFRvIII H score versus overall response as of the cutoff date of 1 Jul. 2019 (15 patients; PD: partial response, SD: stable disease, PR: partial response, Unknown: Not evaluable). A range of EGFRvIII protein expression was observed across tumors with a median H-score of 115. Tumors from 7 patients had H-scores≥100, and 5 tumors had H-scores<100. According to the IHC results, EGFRvIII localized primarily to the cytosolic and membranous compartments of tumor cells, with stromal and immune cells negative for staining. FIG. 6B provides updated results from 29 subjects as of the cutoff date of 13 Aug. 2020.

SEQUENCES

| SEQ | | |
|---|---|---|
| ID | Description | sequence |
| 1 | Human EFGRvIII | LEEKKGNYVVTDHGSCVRACGADSYEMEEDGVRKCKKCEGPCRKVCNGIGIGE |
| | | FKDSLSINATNIKHFKNCTSISGDLHILPVAFRGDSFTHTPPLDPQELDILKT |
| | | VKEITGFLLIQAWPENRTDLHAFENLEIIRGRTKQHGQFSLAVVSLNITSLGL |
| | | RSLKEISDGDVIISGNKNLCYANTINWKKLFGTSGQKTKIISNRGENSCKATG |
| | | QVCHALCSPEGCWGPEPRDCVSCRNVSRGRECVDKCNLLEGEPREFVENSECI |
| | | QCHPECLPQAMNITCTGRGPDNCIQCAHYIDGPHCVKTCPAGVMGENNTLVWK |
| | | YADAGHVCHLCHPNCTYGCTGPGLEGCPTNGPKIPSIATGMVGALLLLLVVAL |
| | | GIGLFMRRRHIVRKRTLRRLLQERELVEPLTPSGEAPNQALLRILKETEFKKI |
| | | KVLGSGAFGTVYKGLWIPEGEKVKIPVAIKELREATSPKANKEILDEAYVMAS |
| | | VDNPHVCRLLGICLTSTVQLITQLMPFGCLLDYVREHKDNIGSQYLLNWCVQI |
| | | AKGMNYLEDRRLVHRDLAARNVLVKTPQHVKITDFGLAKLLGAEEKEYHAEGG |
| | | KVPIKWMALESILHRIYTHQSDVWSYGVTVWELMTFGSKPYDGIPASEISSIL |
| | | EKGERLPQPPICTIDVYMIMVKCWMIDADSRPKFRELIIEFSKMARDPQRYLV |
| | | IQGDERMHLPSPTDSNFYRALMDEEDMDDVVDADEYLIPQQGFFSSPSTSRTP |
| | | LLSSLSATSNNSTVACIDRNGLQSCPIKEDSFLQRYSSDPTGALTEDSIDDTF |
| | | LPVPEYINQSVPKRPAGSVQNPVYHNQPLNPAPSRDPHYQDPHSTAVGNPEYL |
| | | NTVQPTCVNSTFDSPAHWAQKGSHQISLDNPDYQQDFFPKEAKPNGIFKGSTA |
| | | ENAEYLRVAPQSSEFIGA |
| 2 | Cynomolgus | LEEKKGNYVVTDHGSCVRACGADSYEMEEDGVRKCKKCEGPCRKVCNGIGIGE |
| | EGFRvIII | FKDTLSINATNIKHFKNCTSISGDLHILPVAFRGDSFTHTPPLDPQELDILKT |
| | | VKEITGFLLIQAWPENRTDLHAFENLEIIRGRTKQHGQFSLAVVSLNITSLGL |
| | | RSLKEISDGDVIISGNKNLCYANTINWKKLFGTSSQKTKIISNRGENSCKATG |
| | | QVCHALCSPEGCWGPEPRDCVSCQNVSRGRECVDKCNILEGEPREFVENSECI |
| | | QCHPECLPQVMNITCTGRGPDNCIQCAHYIDGPHCVKTCPAGVMGENNTLVWK |
| | | YADAGHVCHLCHPNCTYGCTGPGLEGCARNGPKIPSIATGMLGALLLLLVVAL |
| | | GIGLFMRRRHIVRKRTLRRLLQERELVEPLTPSGEAPNQALLRILKETEFKKI |
| | | KVLGSGAFGTVYKGLWIPEGEKVKIPVAIKELREATSPKANKEILDEAYVMAS |
| | | VDNPHVCRLLGICLTSTVQLITQLMPFGCLLDYVREHKDNIGSQYLLNWCVQI |
| | | AKGMNYLEDRRLVHRDLAARNVLVKTPQHVKITDFGLAKLLGAEEKEYHAEGG |
| | | KVPIKWMALESILHRIYTHQSDVWSYGVTVWELMTFGSKPYDGIPASEISSIL |
| | | EKGERLPQPPICTIDVYMIMVKCWMIDADSRPKFRELIIEFSKMARDPQRYLV |
| | | IQGDERMHLPSPTDSNFYRALMDEEDMDDVVDADEYLIPQQGFFSSPSTSRTP |
| | | LLSSLSATSNNSTVACIDRNGLQSCPIKEDSFLQRYSSDPTGALTEDSIDDTF |
| | | LPVPEYINQSVPKRPAGSVQNPVYHNQPLNPAPSRDPHYQDPHSTAVGNPEYL |
| | | NTVQPTCVNSTFDSPAHWAQKGSHQISLDNPDYQQDFFPKEAKPNGIFKGSTA |
| | | ENAEYLRVAPQSSEFIGA |
| 3 | EGFRvIII- | NYGMH |
| | binding domain | |
| | CDR-H1 | |
| 4 | EGFRVIII- | VIWYDGSDKYYADSVRG |
| | binding domain | |
| | CDR-H2 | |
| 5 | EGFRVIII- | DGYDILTGNPRDFDY |
| | binding domain | |
| | CDR-H3 | |
| 6 | EGFRvIII- | RSSQSLVHSDGNTYLS |
| | binding domain | |
| | CDR-L1 | |
| 7 | EGFRVIII- | RISRRFS |
| | binding domain | |
| | CDR-L2 | |
| 8 | EGFRVIII- | MQSTHVPRT |
| | binding domain | |
| | CDR-L3 | |
| 9 | EGFRVIII- | QVQLVESGGGVVQSGRSLRLSCAASGFTFRNYGMHWVRQAPGKCLEWVAVIWY |
| | binding domain | DGSDKYYADSVRGRFTISRDNSKNTLYLQMNSLRAEDTAVYYCARDGYDILTG |
| | VH | NPRDFDYWGQGTLVTVSS |
| 10 | EGFRVIII- | DTVMTQTPLSSHVTLGQPASISCRSSQSLVHSDGNTYLSWLQQRPGQPPRLLI |
| | binding domain | YRISRRFSGVPDRFSGSGAGTDFTLEISRVEAEDVGVYYCMQSTHVPRTFGCG |
| | VL | TKVEIK |
| 11 | EGFRVIII- | QVQLVESGGGVVQSGRSLRLSCAASGFTFRNYGMHWVRQAPGKCLEWVAVIWY |
| | binding domain | DGSDKYYADSVRGRFTISRDNSKNTLYLQMNSLRAEDTAVYYCARDGYDILTG |
| | scFv | NPRDFDYWGQGTLVTVSSGGGGSGGGGSGGGGSDTVMTQTPLSSHVTLGQPAS |
| | | ISCRSSQSLVHSDGNTYLSWLQQRPGQPPRLLIYRISRRFSGVPDRFSGSGAG |
| | | TDFTLEISRVEAEDVGVYYCMQSTHVPRTFGCGTKVEIK |
| 12 | EGFRvIII-CD3 | QVQLVESGGGVVQSGRSLRLSCAASGFTFRNYGMHWVRQAPGKCLEWVAVIWY |
| | bispecific | DGSDKYYADSVRGRFTISRDNSKNTLYLQMNSLRAEDTAVYYCARDGYDILTG |
| | molecule | NPRDFDYWGQGTLVTVSSGGGGSGGGGSGGGGSDTVMTQTPLSSHVTLGQPAS |
| | | ISCRSSQSLVHSDGNTYLSWLQQRPGQPPRLLIYRISRRFSGVPDRFSGSGAG |
| | | TDFTLEISRVEAEDVGVYYCMQSTHVPRTFGCGTKVEIKSGGGGSEVQLVESG |
| | | GGLVQPGGSLKLSCAASGFTFNKYAMNWVRQAPGKGLEWVARIRSKYNNYATY |
| | | YADSVKDRFTISRDDSKNTAYLQMNNLKTEDTAVYYCVRHGNFGNSYISYWAY |
| | | WGQGTLVTVSSGGGGSGGGGSGGGGSQTVVTQEPSLTVSPGGTVTLTCGSSTG |
| | | AVTSGNYPNWVQQKPGQAPRGLIGGTKFLAPGTPARFSGSLLGGKAALTLSGV |
| | | QPEDEAEYYCVLWYSNRWVFGGGTKLTVL |
| 13 | EGFRvIII-CD3 | QVQLVESGGGVVQSGRSLRLSCAASGFTFRNYGMHWVRQAPGKCLEWVAVIWY |
| | bispecific | DGSDKYYADSVRGRFTISRDNSKNTLYLQMNSLRAEDTAVYYCARDGYDILTG |
| | molecule with | NPRDFDYWGQGTLVTVSSGGGGSGGGGSGGGGSDTVMTQTPLSSHVTLGQPAS |
| | his tag | ISCRSSQSLVHSDGNTYLSWLQQRPGQPPRLLIYRISRRFSGVPDRFSGSGAG |
| | | TDFTLEISRVEAEDVGVYYCMQSTHVPRTFGCGTKVEIKSGGGGSEVQLVESG |
| | | GGLVQPGGSLKLSCAASGFTFNKYAMNWVRQAPGKGLEWVARIRSKYNNYATY |
| | | YADSVKDRFTISRDDSKNTAYLQMNNLKTEDTAVYYCVRHGNFGNSYISYWAY |
| | | WGQGTLVTVSSGGGGSGGGGSGGGGSQTVVTQEPSLTVSPGGTVTLTCGSSTG |
| | | AVTSGNYPNWVQQKPGQAPRGLIGGTKFLAPGTPARFSGSLLGGKAALTLSGV |
| | | QPEDEAEYYCVLWYSNRWVFGGGTKLTVLHHHHHH |
| 14 | Hexa-histidine | HHHHHH |
| 15 | CD3-binding | GSSTGAVTSGYYPN |
| | domain | |
| | CDR-L1 of F6A | |
| 16 | CD3-binding | GTKFLAP |
| | domain | |
| | CDR-L2 of F6A | |
| 17 | CD3-binding | ALWYSNRWV |
| | domain | |
| | CDR-L3 of F6A | |
| 18 | CD3-binding | IYAMN |
| | domain | |
| | CDR-H1 of F6A | |
| 19 | CD3-binding | RIRSKYNNYATYYADSVKS |
| | domain | |
| | CDR-H2 of F6A | |
| 20 | CD3-binding | HGNFGNSYVSFFAY |
| | domain | |
| | CDR-H3 of F6A | |
| 21 | CD3-binding | EVQLVESGGGLVQPGGSLKLSCAASGFTFNIYAMNWVRQAPGKGLEWVARIRS |
| | domain | KYNNYATYYADSVKSRFTISRDDSKNTAYLQMNNLKTEDTAVYYCVRHGNFGN |
| | VH of F6A | SYVSFFAYWGQGTLVTVSS |
| 22 | CD3-binding | QTVVTQEPSLTVSPGGTVTLTCGSSTGAVTSGYYPNWVQQKPGQAPRGLIGGT |
| | domain | KFLAPGTPARFSGSLLGGKAALTLSGVQPEDEAEYYCALWYSNRWVFGGGTKL |
| | VL of F6A | TVL |
| 23 | CD3-binding | EVQLVESGGGLVQPGGSLKLSCAASGFTFNIYAMNWVRQAPGKGLEWVARIRS |
| | domain | KYNNYATYYADSVKSRFTISRDDSKNTAYLQMNNLKTEDTAVYYCVRHGNFGN |
| | scFv of F6A | SYVSFFAYWGQGTLVTVSSGGGGSGGGGSGGGGSQTVVTQEPSLTVSPGGTVT |
| | | LTCGSSTGAVTSGYYPNWVQQKPGQAPRGLIGGTKFLAPGTPARFSGSLLGGK |
| | | AALTLSGVQPEDEAEYYCALWYSNRWVFGGGTKLTVL |
| 24 | CD3-binding | GSSTGAVTSGYYPN |
| | domain | |
| | CDR-L1 of H2C | |
| 25 | CD3-binding | GTKFLAP |
| | domain | |
| | CDR-L2 of H2C | |
| 26 | CD3-binding | ALWYSNRWV |
| | domain | |
| | CDR-L3 of H2C | |
| 27 | CD3-binding | KYAMN |
| | domain | |
| | CDR-H1 of H2C | |
| 28 | CD3-binding | RIRSKYNNYATYYADSVKD |
| | domain | |
| | CDR-H2 of H2C | |
| 29 | CD3-binding | HGNFGNSYISYWAY |
| | domain | |
| | CDR-H3 of H2C | |
| 30 | CD3-binding | EVQLVESGGGLVQPGGSLKLSCAASGFTFNKYAMNWVRQAPGKGLEWVARIRS |
| | domain | KYNNYATYYADSVKDRFTISRDDSKNTAYLQMNNLKTEDTAVYYCVRHGNFGN |
| | VH of H2C | SYISYWAYWGQGTLVTVSS |
| 31 | CD3-binding | QTVVTQEPSLTVSPGGTVTLTCGSSTGAVTSGYYPNWVQQKPGQAPRGLIGGT |
| | domain | KFLAPGTPARFSGSLLGGKAALTLSGVQPEDEAEYYCALWYSNRWVFGGGTKL |
| | VL of H2C | TVL |
| 32 | CD3-binding | EVQLVESGGGLVQPGGSLKLSCAASGFTFNKYAMNWVRQAPGKGLEWVARIRS |
| | domain | KYNNYATYYADSVKDRFTISRDDSKNTAYLQMNNLKTEDTAVYYCVRHGNFGN |
| | scFv of H2C | SYISYWAYWGQGTLVTVSSGGGGSGGGGSGGGGSQTVVTQEPSLTVSPGGTVT |
| | | LTCGSSTGAVTSGYYPNWVQQKPGQAPRGLIGGTKFLAPGTPARFSGSLLGGK |
| | | AALTLSGVQPEDEAEYYCALWYSNRWVFGGGTKLTVL |
| 33 | CD3-binding | GSSTGAVTSGYYPN |
| | domain | |
| | CDR-L1 of H1E | |
| 34 | CD3-binding | GTKFLAP |
| | domain | |
| | CDR-L2 of H1E | |
| 35 | CD3-binding | ALWYSNRWV |
| | domain | |
| | CDR-L3 of H1E | |
| 36 | CD3-binding | SYAMN |
| | domain | |
| | CDR-H1 of H1E | |
| 37 | CD3-binding | RIRSKYNNYATYYADSVKG |
| | domain | |
| | CDR-H2 of H1E | |
| 38 | CD3-binding | HGNFGNSYLSFWAY |
| | domain | |
| | CDR-H3 of H1E | |
| 39 | CD3-binding | EVQLVESGGGLEQPGGSLKLSCAASGFTFNSYAMNWVRQAPGKGLEWVARIRS |
| | domain | KYNNYATYYADSVKGRFTISRDDSKNTAYLQMNNLKTEDTAVYYCVRHGNFGN |
| | VH of H1E | SYLSFWAYWGQGTLVTVSS |
| 40 | CD3-binding | QTVVTQEPSLTVSPGGTVTLTCGSSTGAVTSGYYPNWVQQKPGQAPRGLIGGT |
| | domain | KFLAPGTPARFSGSLLGGKAALTLSGVQPEDEAEYYCALWYSNRWVFGGGTKL |
| | VL of H1E | TVL |
| 41 | CD3-binding | EVQLVESGGGLEQPGGSLKLSCAASGFTFNSYAMNWVRQAPGKGLEWVARIRS |
| | domain | KYNNYATYYADSVKGRFTISRDDSKNTAYLQMNNLKTEDTAVYYCVRHGNFGN |
| | scFv of H1E | SYLSFWAYWGQGTLVTVSSGGGGSGGGGSGGGGSQTVVTQEPSLTVSPGGTVT |
| | | LTCGSSTGAVTSGYYPNWVQQKPGQAPRGLIGGTKFLAPGTPARFSGSLLGGK |
| | | AALTLSGVQPEDEAEYYCALWYSNRWVFGGGTKLTVL |
| 42 | CD3-binding | GSSTGAVTSGYYPN |
| | domain | |
| | CDR-L1 of G4H | |
| 43 | CD3-binding | GTKFLAP |
| | domain | |
| | CDR-L2 of G4H | |
| 44 | CD3-binding | ALWYSNRWV |
| | domain | |
| | CDR-L3 of G4H | |
| 45 | CD3-binding | RYAMN |
| | domain | |
| | CDR-H1 of G4H | |
| 46 | CD3-binding | RIRSKYNNYATYYADSVKG |
| | domain | |
| | CDR-H2 of G4H | |
| 47 | CD3-binding | HGNFGNSYLSYFAY |
| | domain | |
| | CDR-H3 of G4H | |
| 48 | CD3-binding | EVQLVESGGGLVQPGGSLKLSCAASGFTFNRYAMNWVRQAPGKGLEWVARIRS |
| | domain | KYNNYATYYADSVKGRFTISRDDSKNTAYLQMNNLKTEDTAVYYCVRHGNFGN |
| | VH of G4H | SYLSYFAYWGQGTLVTVSS |
| 49 | CD3-binding | QTVVTQEPSLTVSPGGTVTLTCGSSTGAVTSGYYPNWVQQKPGQAPRGLIGGT |
| | domain | KFLAPGTPARFSGSLLGGKAALTLSGVQPEDEAEYYCALWYSNRWVFGGGTKL |
| | VL of G4H | TVL |
| 50 | CD3-binding | EVQLVESGGGLVQPGGSLKLSCAASGFTFNRYAMNWVRQAPGKGLEWVARIRS |
| | domain | KYNNYATYYADSVKGRFTISRDDSKNTAYLQMNNLKTEDTAVYYCVRHGNFGN |
| | scFv of G4H | SYLSYFAYWGQGTLVTVSSGGGGSGGGGSGGGGSQTVVTQEPSLTVSPGGTVT |
| | | LTCGSSTGAVTSGYYPNWVQQKPGQAPRGLIGGTKFLAPGTPARFSGSLLGGK |
| | | AALTLSGVQPEDEAEYYCALWYSNRWVFGGGTKLTVL |
| 51 | CD3-binding | RSSTGAVTSGYYPN |
| | domain | |
| | CDR-L1 of A2J | |
| 52 | CD3-binding | ATDMRPS |
| | domain | |
| | CDR-L2 of A2J | |
| 53 | CD3-binding | ALWYSNRWV |
| | domain | |
| | CDR-L3 of A2J | |
| 54 | CD3-binding | VYAMN |
| | domain | |
| | CDR-H1 of A2J | |
| 55 | CD3-binding | RIRSKYNNYATYYADSVKK |
| | domain | |
| | CDR-H2 of A2J | |
| 56 | CD3-binding | HGNFGNSYLSWWAY |
| | domain | |
| | CDR-H3 of A2J | |
| 57 | CD3-binding | EVQLVESGGGLVQPGGSLKLSCAASGFTFNVYAMNWVRQAPGKGLEWVARIRS |
| | domain | KYNNYATYYADSVKKRFTISRDDSKNTAYLQMNNLKTEDTAVYYCVRHGNFGN |
| | VH of A2J | SYLSWWAYWGQGTLVTVSS |
| 58 | CD3-binding | QTVVTQEPSLTVSPGGTVTLTCRSSTGAVTSGYYPNWVQQKPGQAPRGLIGAT |
| | domain | DMRPSGTPARFSGSLLGGKAALTLSGVQPEDEAEYYCALWYSNRWVFGGGTKL |
| | VL of A2J | TVL |
| 59 | CD3-binding | EVQLVESGGGLVQPGGSLKLSCAASGFTFNVYAMNWVRQAPGKGLEWVARIRS |
| | domain | KYNNYATYYADSVKKRFTISRDDSKNTAYLQMNNLKTEDTAVYYCVRHGNFGN |
| | scFv of A2J | SYLSWWAYWGQGTLVTVSSGGGGSGGGGSGGGGSQTVVTQEPSLTVSPGGTVT |
| | | LTCRSSTGAVTSGYYPNWVQQKPGQAPRGLIGATDMRPSGTPARFSGSLLGGK |
| | | AALTLSGVQPEDEAEYYCALWYSNRWVFGGGTKLTVL |
| 60 | CD3-binding | GSSTGAVTSGYYPN |
| | domain | |
| | CDR-L1 of E1L | |
| 61 | CD3-binding | GTKFLAP |
| | domain | |
| | CDR-L2 of E1L | |
| 62 | CD3-binding | ALWYSNRWV |
| | domain | |
| | CDR-L3 of E1L | |
| 63 | CD3-binding | KYAMN |
| | domain | |
| | CDR-H1 of E1L | |
| 64 | CD3-binding | RIRSKYNNYATYYADSVKS |
| | domain | |
| | CDR-H2 of E1L | |
| 65 | CD3-binding | HGNFGNSYTSYYAY |
| | domain | |
| | CDR-H3 of E1L | |
| 66 | CD3-binding | EVQLVESGGGLVQPGGSLKLSCAASGFTFNKYAMNWVRQAPGKGLEWVARIRS |
| | domain | KYNNYATYYADSVKSRFTISRDDSKNTAYLQMNNLKTEDTAVYYCVRHGNFGN |
| | VH of E1L | SYTSYYAYWGQGTLVTVSS |
| 67 | CD3-binding | QTVVTQEPSLTVSPGGTVTLTCGSSTGAVTSGYYPNWVQQKPGQAPRGLIGGT |
| | domain | KFLAPGTPARFSGSLLGGKAALTLSGVQPEDEAEYYCALWYSNRWVFGGGTKL |
| | VL of E1L | TVL |
| 68 | CD3-binding | EVQLVESGGGLVQPGGSLKLSCAASGFTFNKYAMNWVRQAPGKGLEWVARIRS |
| | domain | KYNNYATYYADSVKSRFTISRDDSKNTAYLQMNNLKTEDTAVYYCVRHGNFGN |
| | scFv of E1L | SYTSYYAYWGQGTLVTVSSGGGGSGGGGSGGGGSQTVVTQEPSLTVSPGGTVT |
| | | LTCGSSTGAVTSGYYPNWVQQKPGQAPRGLIGGTKFLAPGTPARFSGSLLGGK |
| | | AALTLSGVQPEDEAEYYCALWYSNRWVFGGGTKLTVL |
| 69 | CD3-binding | RSSTGAVTSGYYPN |
| | domain | |
| | CDR-L1 of E2M | |
| 70 | CD3-binding | ATDMRPS |
| | domain | |
| | CDR-L2 of E2M | |
| 71 | CD3-binding | ALWYSNRWV |
| | domain | |
| | CDR-L3 of E2M | |
| 72 | CD3-binding | GYAMN |
| | domain | |
| | CDR-H1 of E2M | |
| 73 | CD3-binding | RIRSKYNNYATYYADSVKE |
| | domain | |
| | CDR-H2 of E2M | |
| 74 | CD3-binding | HRNFGNSYLSWFAY |
| | domain | |
| | CDR-H3 of E2M | |
| 75 | CD3-binding | EVQLVESGGGLVQPGGSLKLSCAASGFTFNGYAMNWVRQAPGKGLEWVARIRS |
| | domain | KYNNYATYYADSVKERFTISRDDSKNTAYLQMNNLKTEDTAVYYCVRHRNFGN |
| | VH of E2M | SYLSWFAYWGQGTLVTVSS |
| 76 | CD3-binding | QTVVTQEPSLTVSPGGTVTLTCRSSTGAVTSGYYPNWVQQKPGQAPRGLIGAT |
| | domain | DMRPSGTPARFSGSLLGGKAALTLSGVQPEDEAEYYCALWYSNRWVFGGGTKL |
| | VL of E2M | TVL |
| 77 | CD3-binding | EVQLVESGGGLVQPGGSLKLSCAASGFTFNGYAMNWVRQAPGKGLEWVARIRS |
| | domain | KYNNYATYYADSVKERFTISRDDSKNTAYLQMNNLKTEDTAVYYCVRHRNFGN |
| | scFv of E2M | SYLSWFAYWGQGTLVTVSSGGGGSGGGGSGGGGSQTVVTQEPSLTVSPGGTVT |
| | | LTCRSSTGAVTSGYYPNWVQQKPGQAPRGLIGATDMRPSGTPARFSGSLLGGK |
| | | AALTLSGVQPEDEAEYYCALWYSNRWVFGGGTKLTVL |
| 78 | CD3-binding | GSSTGAVTSGYYPN |
| | domain | |
| | CDR-L1 of F70 | |
| 79 | CD3-binding | GTKFLAP |
| | domain | |
| | CDR-L2 of F70 | |
| 80 | CD3-binding | ALWYSNRWV |
| | domain | |
| | CDR-L3 of F70 | |
| 81 | CD3-binding | VYAMN |
| | domain | |
| | CDR-H1 of F70 | |
| 82 | CD3-binding | RIRSKYNNYATYYADSVKK |
| | domain | |
| | CDR-H2 of F70 | |
| 83 | CD3-binding | HGNFGNSYISWWAY |
| | domain | |
| | CDR-H3 of F70 | |
| 84 | CD3-binding | EVQLVESGGGLVQPGGSLKLSCAASGFTFNVYAMNWVRQAPGKGLEWVARIRS |
| | domain | KYNNYATYYADSVKKRFTISRDDSKNTAYLQMNNLKTEDTAVYYCVRHGNFGN |
| | VH of F70 | SYISWWAYWGQGTLVTVSS |
| 85 | CD3-binding | QTVVTQEPSLTVSPGGTVTLTCGSSTGAVTSGYYPNWVQQKPGQAPRGLIGGT |
| | domain | KFLAPGTPARFSGSLLGGKAALTLSGVQPEDEAEYYCALWYSNRWVFGGGTKL |
| | VL of F70 | TVL |
| 86 | CD3-binding | EVQLVESGGGLVQPGGSLKLSCAASGFTFNVYAMNWVRQAPGKGLEWVARIRS |
| | domain | KYNNYATYYADSVKKRFTISRDDSKNTAYLQMNNLKTEDTAVYYCVRHGNFGN |
| | scFv of F70 | SYISWWAYWGQGTLVTVSSGGGGSGGGGSGGGGSQTVVTQEPSLTVSPGGTVT |
| | | LTCGSSTGAVTSGYYPNWVQQKPGQAPRGLIGGTKFLAPGTPARFSGSLLGGK |
| | | AALTLSGVQPEDEAEYYCALWYSNRWVFGGGTKLTVL |
| 87 | CD3-binding | GSSTGAVTSGNYPN |
| | domain | |
| | CDR-L1 of F12Q | |
| 88 | CD3-binding | GTKFLAP |
| | domain | |
| | CDR-L2 of F12Q | |
| 89 | CD3-binding | VLWYSNRWV |
| | domain | |
| | CDR-L3 of F12Q | |
| 90 | CD3-binding | SYAMN |
| | domain | |
| | CDR-H1 of F12Q | |
| 91 | CD3-binding | RIRSKYNNYATYYADSVKG |
| | domain | |
| | CDR-H2 of F12Q | |
| 92 | CD3-binding | HGNFGNSYVSWWAY |
| | domain | |
| | CDR-H3 of F12Q | |
| 93 | CD3-binding | EVQLVESGGGLVQPGGSLKLSCAASGFTFNSYAMNWVRQAPGKGLEWVARIRS |
| | domain | KYNNYATYYADSVKGRFTISRDDSKNTAYLQMNNLKTEDTAVYYCVRHGNFGN |
| | VH of F12Q | SYVSWWAYWGQGTLVTVSS |
| 94 | CD3-binding | QTVVTQEPSLTVSPGGTVTLTCGSSTGAVTSGNYPNWVQQKPGQAPRGLIGGT |
| | domain | KFLAPGTPARFSGSLLGGKAALTLSGVQPEDEAEYYCVLWYSNRWVFGGGTKL |
| | VL of F12Q | TVL |
| 95 | CD3-binding | EVQLVESGGGLVQPGGSLKLSCAASGFTFNSYAMNWVRQAPGKGLEWVARIRS |
| | domain | KYNNYATYYADSVKGRFTISRDDSKNTAYLQMNNLKTEDTAVYYCVRHGNFGN |
| | scFv of F12Q | SYVSWWAYWGQGTLVTVSSGGGGSGGGGSGGGGSQTVVTQEPSLTVSPGGTVT |
| | | LTCGSSTGAVTSGNYPNWVQQKPGQAPRGLIGGTKFLAPGTPARFSGSLLGGK |
| | | AALTLSGVQPEDEAEYYCVLWYSNRWVFGGGTKLTVL |
| 96 | CD3-binding | GSSTGAVTSGNYPN |
| | domain | |
| | CDR-L1 of I2C | |
| 97 | CD3-binding | GTKFLAP |
| | domain | |
| | CDR-L2 of I2C | |
| 98 | CD3-binding | VLWYSNRWV |
| | domain | |
| | CDR-L3 of I2C | |
| 99 | CD3-binding | KYAMN |
| | domain | |
| | CDR-H1 of I2C | |
| 100 | CD3-binding | RIRSKYNNYATYYADSVKD |
| | domain | |
| | CDR-H2 of I2C | |
| 101 | CD3-binding | HGNFGNSYISYWAY |
| | domain | |
| | CDR-H3 of I2C | |
| 102 | CD3-binding | EVQLVESGGGLVQPGGSLKLSCAASGFTFNKYAMNWVRQAPGKGLEWVARIRS |
| | domain | KYNNYATYYADSVKDRFTISRDDSKNTAYLQMNNLKTEDTAVYYCVRHGNFGN |
| | VH of I2C | SYISYWAYWGQGTLVTVSS |
| 103 | CD3-binding | QTVVTQEPSLTVSPGGTVTLTCGSSTGAVTSGNYPNWVQQKPGQAPRGLIGGT |
| | domain | KFLAPGTPARFSGSLLGGKAALTLSGVQPEDEAEYYCVLWYSNRWVFGGGTKL |
| | VL of I2C | TVL |
| 104 | CD3-binding | EVQLVESGGGLVQPGGSLKLSCAASGFTFNKYAMNWVRQAPGKGLEWVARIRS |
| | domain | KYNNYATYYADSVKDRFTISRDDSKNTAYLQMNNLKTEDTAVYYCVRHGNFGN |
| | scFv of I2C | SYISYWAYWGQGTLVTVSSGGGGSGGGGSGGGGSQTVVTQEPSLTVSPGGTVT |
| | | LTCGSSTGAVTSGNYPNWVQQKPGQAPRGLIGGTKFLAPGTPARFSGSLLGGK |
| | | AALTLSGVQPEDEAEYYCVLWYSNRWVFGGGTKLTVL |
| 105 | CD3-binding | GSSTGAVTSGNYPN |
| | domain | |
| | CDR-L1 of F12q | |
| 106 | CD3-binding | GTKFLAP |
| | domain | |
| | CDR-L2 of F12q | |
| 107 | CD3-binding | VLWYSNRWV |
| | domain | |
| | CDR-L3 of F12q | |
| 108 | CD3-binding | SYAMN |
| | domain | |
| | CDR-H1 of F12q | |
| 109 | CD3-binding | RIRSKYNNYATYYADSVKG |
| | domain | |
| | CDR-H2 of F12q | |
| 110 | CD3-binding | HGNFGNSYVSWWAY |
| | domain | |
| | CDR-H3 of F12q | |
| 111 | CD3-binding | EVQLVESGGGLVQPGGSLRLSCAASGFTFNSYAMNWVRQAPGKGLEWVARIRS |
| | domain | KYNNYATYYADSVKGRFTISRDDSKNTAYLQMNSLKTEDTAVYYCVRHGNFGN |
| | VH of F12q | SYVSWWAYWGQGTLVTVSS |
| 112 | CD3-binding | QTVVTQEPSLTVSPGGTVTLTCGSSTGAVTSGNYPNWVQQKPGQAPRGLIGGT |
| | domain | KFLAPGTPARFSGSLLGGKAALTLSGVQPEDEAEYYCVLWYSNRWVFGGGTKL |
| | VL of F12q | TVL |
| 113 | CD3-binding | EVQLVESGGGLVQPGGSLRLSCAASGFTFNSYAMNWVRQAPGKGLEWVARIRS |
| | domain | KYNNYATYYADSVKGRFTISRDDSKNTAYLQMNSLKTEDTAVYYCVRHGNFGN |
| | scFv of F12q | SYVSWWAYWGQGTLVTVSSGGGGSGGGGSGGGGSQTVVTQEPSLTVSPGGTVT |
| | | LTCGSSTGAVTSGNYPNWVQQKPGQAPRGLIGGTKFLAPGTPARFSGSLLGGK |
| | | AALTLSGVQPEDEAEYYCVLWYSNRWVFGGGTKLTVL |
| 114 | Peptide linker | GGGG |
| 115 | Peptide linker | GGGGS |
| 116 | Peptide linker | GGGGQ |
| 117 | Peptide linker | PGGGGS |
| 118 | Peptide linker | PGGDGS |
| 119 | Peptide linker | SGGGGS |
| 120 | Peptide linker | GGGGSGGGS |
| 121 | Peptide linker | GGGGSGGGGS |
| 122 | Peptide linker | GGGGSGGGGSGGGGS |
| 123 | Human CD3 | QDGNEEMGGI TQTPYKVSIS GTTVILTCPQ YPGSEILWQH |
| | epsilon | NDKNIGGDED DKNIGSDEDH LSLKEFSELE QSGYYVCYPR |
| | extracellular | GSKPEDANFY LYLRARVCEN CMEMD |
| | domain | |
| 124 | Callithrix | QDGNEEMGDT TQNPYKVSIS GTTVTLTCPR YDGHEIKWLV |
| | jacchus CD3 | NSQNKEGHED HLLLEDFSEM EQSGYYACLS KETPAEEASH |
| | epsilon | YLYLKARVCE NCVEVD |
| | extracellular | |
| | domain | |
| 125 | Saguinus | QDGNEEMGDT TQNPYKVSIS GTTVTLTCPR YDGHEIKWLV |
| | oedipus CD3 | NSQNKEGHED HLLLEDFSEM EQSGYYACLS KETPAEEASH |
| | epsilon | YLYLKARVCE NCVEVD |
| | extracellular | |
| | domain | |
| 126 | Saimiri | QDGNEEIGDT TQNPYKVSIS GTTVTLTCPR YDGQEIKWLV |
| | sciureus CD3 | NDQNKEGHED HLLLEDFSEM EQSGYYACLS KETPTEEASH |
| | epsilon | YLYLKARVCE NCVEVD |
| | extracellular | |
| | domain | |
| 127 | EGFRvIII | SYGMH |
| | antibody 1 CDR- | |
| | H1 | |
| 128 | EGFRvIII | VIWYDGSNKYYVDSVKG |
| | antibody 1 CDR- | |
| | H2 | |
| 129 | EGFRvIII | DGWQQLAPFDY |
| | antibody 1 CDR- | |
| | H3 | |
| 130 | EGFRvIII | RSSQSLVHSDGNTYLS |
| | antibody 1 CDR- | |
| | L1 | |
| 131 | EGFRvIII | KISNRFS |
| | antibody 1 CDR- | |
| | L2 | |
| 132 | EGFRvIII | MQATQLPRT |
| | antibody 1 CDR- | |
| | L3 | |
| 133 | EGFRvIII | QVQLVESGGGVVQPGRSLRLSCAASGFTFSSYGMHWVRQAPGKGLEWVAVIWY |
| | antibody 1 VH | DGSNKYYVDSVKGRFTISRDNSKNTLYLQMNSLRAEDTAVYYCARDGWQQLAP |
| | | FDYWGQGTLVTVSA |
| 134 | EGFRvIII | DIVMTQTPLSSPVTLGQPASISCRSSQSLVHSDGNTYLSWLHQRPGQPPRLLI |
| | antibody 1 VL | YKISNRFSGVPDRFSGSGAGTAFTLKISRVEAEDVGVYYCMQATQLPRTFGQG |
| | | TKVEIK |
| 135 | EGFRvIII | AKTTPPSVYPLAPGSAAQTNSMVTLGCLVKGYFPEPVTVTWNSGSLSSGVHTF |
| | antibody 1 CH | PAVLQSDLYTLSSSVTVPSSTWPSETVTCNVAHPASSTKVDKKIVPRDCGCKP |
| | | CICTVPEVSSVFIFPPKPKDVLTITLTPKVTCVVVDISKDDPEVQFSWFVDDV |
| | | EVHTAQTQPREEQFNSTFRSVSELPIMHQDWLNGKEFKCRVNSAAFPAPIEKT |
| | | ISKTKGRPKAPQVYTIPPPKEQMAKDKVSLTCMITDFFPEDITVEWQWNGQPA |
| | | ENYKNTQPIMDTDGSYFVYSKLNVQKSNWEAGNTFTCSVLHEGLHNHHTEKSL |
| | | SHSPGK |
| 136 | EGFRvIII | RADAAPTVSIFPPSSEQLTSGGASVVCFLNNFYPKDINVKWKIDGSERQNGVL |
| | antibody 1 CL | NSWTDQDSKDSTYSMSSTLTLTKDEYERHNSYTCEATHKTSTSPIVKSFNRNE |
| | | C |
| 137 | EGFRvIII | QVQLVESGGGVVQPGRSLRLSCAASGFTFSSYGMHWVRQAPGKGLEWVAVIWY |
| | antibody 1 | DGSNKYYVDSVKGRFTISRDNSKNTLYLQMNSLRAEDTAVYYCARDGWQQLAP |
| | heavy chain | FDYWGQGTLVTVSAAKTTPPSVYPLAPGSAAQTNSMVTLGCLVKGYFPEPVTV |
| | | TWNSGSLSSGVHTFPAVLQSDLYTLSSSVTVPSSTWPSETVTCNVAHPASSTK |
| | | VDKKIVPRDCGCKPCICTVPEVSSVFIFPPKPKDVLTITLTPKVTCVVVDISK |
| | | DDPEVQFSWFVDDVEVHTAQTQPREEQFNSTFRSVSELPIMHQDWLNGKEFKC |
| | | RVNSAAFPAPIEKTISKTKGRPKAPQVYTIPPPKEQMAKDKVSLTCMITDFFP |
| | | EDITVEWQWNGQPAENYKNTQPIMDTDGSYFVYSKLNVQKSNWEAGNTFTCSV |
| | | LHEGLHNHHTEKSLSHSPGK |
| 138 | EGFRvIII | DIVMTQTPLSSPVTLGQPASISCRSSQSLVHSDGNTYLSWLHQRPGQPPRLLI |
| | antibody 1 | YKISNRFSGVPDRFSGSGAGTAFTLKISRVEAEDVGVYYCMQATQLPRTFGQG |
| | light chain | TKVEIKRADAAPTVSIFPPSSEQLTSGGASVVCFLNNFYPKDINVKWKIDGSE |
| | | RQNGVLNSWTDQDSKDSTYSMSSTLTLTKDEYERHNSYTCEATHKTSTSPIVK |
| | | SFNRNEC |
| 139 | EGFRvIII | QVQLVESGGGVVQSGRSLRLSCAASGFTFRNYGMHWVRQAPGKGLEWVAVIWY |
| | antibody 2 VH | DGSDKYYADSVRGRFTISRDNSKNTLYLQMNSLRAEDTAVYYCARDGYDILTG |
| | | NPRDFDYWGQGTLVTVSA |
| 140 | EGFRvIII | DTVMTQTPLSSHVTLGQPASISCRSSQSLVHSDGNTYLSWLQQRPGQPPRLLI |
| | antibody 2 VL | YRISRRFSGVPDRFSGSGAGTDFTLEISRVEAEDVGVYYCMQSTHVPRTFGQG |
| | | TKVEIK |
| 141 | EGFRvIII | QVQLVESGGGVVQSGRSLRLSCAASGFTFRNYGMHWVRQAPGKGLEWVAVIWY |
| | antibody 2 | DGSDKYYADSVRGRFTISRDNSKNTLYLQMNSLRAEDTAVYYCARDGYDILTG |
| | heavy chain | NPRDFDYWGQGTLVTVSAAKTTPPSVYPLAPGSAAQTNSMVTLGCLVKGYFPE |
| | | PVTVTWNSGSLSSGVHTFPAVLQSDLYTLSSSVTVPSSTWPSETVTCNVAHPA |
| | | SSTKVDKKIVPRDCGCKPCICTVPEVSSVFIFPPKPKDVLTITLTPKVTCVVV |
| | | DISKDDPEVQFSWFVDDVEVHTAQTQPREEQFNSTFRSVSELPIMHQDWLNGK |
| | | EFKCRVNSAAFPAPIEKTISKTKGRPKAPQVYTIPPPKEQMAKDKVSLTCMIT |
| | | DFFPEDITVEWQWNGQPAENYKNTQPIMDTDGSYFVYSKLNVQKSNWEAGNTF |
| | | TCSVLHEGLHNHHTEKSLSHSPGK |
| 142 | EGFRvIII | DTVMTQTPLSSHVTLGQPASISCRSSQSLVHSDGNTYLSWLQQRPGQPPRLLI |
| | antibody 2 | YRISRRFSGVPDRFSGSGAGTDFTLEISRVEAEDVGVYYCMQSTHVPRTFGQG |
| | light chain | TKVEIKRADAAPTVSIFPPSSEQLTSGGASVVCFLNNFYPKDINVKWKIDGSE |
| | | RQNGVLNSWTDQDSKDSTYSMSSTLTLTKDEYERHNSYTCEATHKTSTSPIVK |
| | | SFNRNEC |

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. A method of treating EGFRvIII-positive cancer, comprising administering to a subject in need thereof an anti-EGFRvIII agent, at an initial dose of from 15 μg/day to 12000 μg/day, for at least 14 days, wherein said anti-EGFRvIII agent comprises the amino acid sequence of SEQ ID Nos. 9, 10, 102, and 103.

2. The method of claim 1, wherein said EGFRvIII-positive cancer is glioblastoma.

3. The method of claim 1, wherein said anti-EGFRvIII agent comprises the amino acid sequence of SEQ ID Nos. 11 and 104.

4. The method of claim 1, wherein said anti-EGFRvIII agent comprises the amino acid sequence of SEQ ID NO. 12 or SEQ ID NO: 13.

5. The method of claim 1, wherein said anti-EGFRvIII agent is administered at an initial dose of from 1000 μg/day to 6000 μg/day.

6. The method of claim 1, wherein said anti-EGFRvIII agent is administered at an initial dose of from 1500 μg/day to 6000 μg/day.

7. The method of claim 1, wherein said anti-EGFRvIII agent is administered at an initial dose of from 3000 μg/day to 6000 μg/day.

8. The method of claim 1, wherein said anti-EGFRvIII agent is administered by continuous intravenous (cIV) infusion.

9. The method of claim 1, wherein said anti-EGFRvIII agent is administered for at least 28 days at the dose of from 15 μg/day to 12000 μg/day.

10. The method of claim 1, wherein said anti-EGFRvIII agent is administered for at least 28 days at the dose of from 1500 μg/day to 6000 μg/day.

11. The method of claim 1, wherein said anti-EGFRvIII agent is administered for at least 28 days at the dose of from 3000 μg/day to 6000 μg/day.

12. The method of claim 1, further comprising administering to the subject one or more subsequent doses of the anti-EGFRvIII agent, at a dose of from 15 μg/day to 12000 μg/day, for at least 14 days.

13. The method of claim 1, further comprising administering to the subject one or more subsequent doses of the anti-EGFRvIII agent, at a dose of from 3000 μg/day to 6000 μg/day, for at least 14 days.

14. The method of claim 1, further comprising administering to the subject one or more subsequent doses of the anti-EGFRvIII agent, at a dose of from 15 μg/day to 12000 μg/day, for at least 28 days.

15. The method of claim 1, further comprising administering to the subject one or more subsequent doses of the anti-EGFRvIII agent, at a dose of from 3000 μg/day to 6000 μg/day, for at least 28 days.

16. The method of claim 12, wherein said one or more subsequent doses are dosed at least one week after the previous dose.

17. The method of claim 1, wherein said anti-EGFRvIII agent is administered at a 14-day on/14-day off cycle, or a 28-day on/14-day off cycle.

18. The method of claim 1, further comprising administered an anti-inflammatory agent to said subject.

19. The method of claim 18, said anti-inflammatory agent is a corticosteroid.

20. The method of claim 1, further comprising (a) obtaining a biological sample from said subject; and (b) detecting the presence of EGFRvIII or measuring the expression level of EGFRvIII in said sample.

21. The method of claim 1, wherein said subject has been assessed for EGFRvIII-positivity accordingly to H-score.

22. The method of claim 21, wherein said H-score of a biological sample of the subject in need of treatment is from 8 to 280.


SEQUENCE LISTING (144 sequences)

SEQ ID NO: 1 - protein, 919 aa, Homo sapiens
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 2 - protein, 919 aa, Macaca fascicularis
LEEKKGNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPSIATGMLGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 3 - protein, 5 aa, Artificial Sequence
  source: /note=“Description of Artificial Sequence Synthetic peptide”
NYGMH

SEQ ID NO: 4 - protein, 17 aa, Artificial Sequence
  source: /note=“Description of Artificial Sequence Synthetic peptide”
VIWYDGSDKYYADSVRG

SEQ ID NO: 5 - protein, 15 aa, Artificial Sequence
  source: /note=“Description of Artificial Sequence Synthetic peptide”
DGYDILTGNPRDFDY

SEQ ID NO: 6 - protein, 16 aa, Artificial Sequence
  source: /note=“Description of Artificial Sequence Synthetic peptide”
RSSQSLVHSDGNTYLS

SEQ ID NO: 7 - protein, 7 aa, Artificial Sequence
  source: /note=“Description of Artificial Sequence Synthetic peptide”
RISRRFS

SEQ ID NO: 8 - protein, 9 aa, Artificial Sequence
  source: /note=“Description of Artificial Sequence Synthetic peptide”
MQSTHVPRT

SEQ ID NO: 9 - protein, 124 aa, Artificial Sequence
  source: /note=“Description of Artificial Sequence Synthetic polypeptide”
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 10 - protein, 112 aa, Artificial Sequence
  source: /note=“Description of Artificial Sequence Synthetic polypeptide”
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 11 - protein, 251 aa, Artificial Sequence
  source: /note=“Description of Artificial Sequence Synthetic polypeptide”
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 12 - protein, 506 aa, Artificial Sequence
  source: /note=“Description of Artificial Sequence Synthetic polypeptide”
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 13 - protein, 512 aa, Artificial Sequence
  source: /note=“Description of Artificial Sequence Synthetic polypeptide”
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 14 - protein, 6 aa, Artificial Sequence
  source: /note=“Description of Artificial Sequence Synthetic 6xHis tag”
HHHHHH

SEQ ID NO: 15 - protein, 14 aa, Artificial Sequence
  source: /note=“Description of Artificial Sequence Synthetic peptide”
GSSTGAVTSGYYPN

SEQ ID NO: 16 - protein, 7 aa, Artificial Sequence
  source: /note=“Description of Artificial Sequence Synthetic peptide”
GTKFLAP

SEQ ID NO: 17 - protein, 9 aa, Artificial Sequence
  source: /note=“Description of Artificial Sequence Synthetic peptide”
ALWYSNRWV

SEQ ID NO: 18 - protein, 5 aa, Artificial Sequence
  source: /note=“Description of Artificial Sequence Synthetic peptide”
IYAMN

SEQ ID NO: 19 - protein, 19 aa, Artificial Sequence
  source: /note=“Description of Artificial Sequence Synthetic peptide”
RIRSKYNNYATYYADSVKS

SEQ ID NO: 20 - protein, 14 aa, Artificial Sequence
  source: /note=“Description of Artificial Sequence Synthetic peptide”
HGNFGNSYVSFFAY

SEQ ID NO: 21 - protein, 125 aa, Artificial Sequence
  source: /note=“Description of Artificial Sequence Synthetic polypeptide”
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 22 - protein, 109 aa, Artificial Sequence
  source: /note=“Description of Artificial Sequence Synthetic polypeptide”
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 23 - protein, 249 aa, Artificial Sequence
  source: /note=“Description of Artificial Sequence Synthetic polypeptide”
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 24 - protein, 14 aa, Artificial Sequence
  source: /note=“Description of Artificial Sequence Synthetic peptide”
GSSTGAVTSGYYPN

SEQ ID NO: 25 - protein, 7 aa, Artificial Sequence
  source: /note=“Description of Artificial Sequence Synthetic peptide”
GTKFLAP

SEQ ID NO: 26 - protein, 9 aa, Artificial Sequence
  source: /note=“Description of Artificial Sequence Synthetic peptide”
ALWYSNRWV

SEQ ID NO: 27 - protein, 5 aa, Artificial Sequence
  source: /note=“Description of Artificial Sequence Synthetic peptide”
KYAMN

SEQ ID NO: 28 - protein, 19 aa, Artificial Sequence
  source: /note=“Description of Artificial Sequence Synthetic peptide”
RIRSKYNNYATYYADSVKD

SEQ ID NO: 29 - protein, 14 aa, Artificial Sequence
  source: /note=“Description of Artificial Sequence Synthetic peptide”
HGNFGNSYISYWAY

SEQ ID NO: 30 - protein, 125 aa, Artificial Sequence
  source: /note=“Description of Artificial Sequence Synthetic polypeptide”
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 31 - protein, 109 aa, Artificial Sequence; identical to SEQ ID NO: 22
  source: /note=“Description of Artificial Sequence Synthetic polypeptide”

SEQ ID NO: 32 - protein, 249 aa, Artificial Sequence
  source: /note=“Description of Artificial Sequence Synthetic polypeptide”
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 33 - protein, 14 aa, Artificial Sequence
  source: /note=“Description of Artificial Sequence Synthetic peptide”
GSSTGAVTSGYYPN

SEQ ID NO: 34 - protein, 7 aa, Artificial Sequence
  source: /note=“Description of Artificial Sequence Synthetic peptide”
GTKFLAP

SEQ ID NO: 35 - protein, 9 aa, Artificial Sequence
  source: /note=“Description of Artificial Sequence Synthetic peptide”
ALWYSNRWV

SEQ ID NO: 36 - protein, 5 aa, Artificial Sequence
  source: /note=“Description of Artificial Sequence Synthetic peptide”
SYAMN

SEQ ID NO: 37 - protein, 19 aa, Artificial Sequence
  source: /note=“Description of Artificial Sequence Synthetic peptide”
RIRSKYNNYATYYADSVKG

SEQ ID NO: 38 - protein, 14 aa, Artificial Sequence
  source: /note=“Description of Artificial Sequence Synthetic peptide”
HGNFGNSYLSFWAY

SEQ ID NO: 39 - protein, 125 aa, Artificial Sequence
  source: /note=“Description of Artificial Sequence Synthetic polypeptide”
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 40 - protein, 109 aa, Artificial Sequence; identical to SEQ ID NO: 22
  source: /note=“Description of Artificial Sequence Synthetic polypeptide”

SEQ ID NO: 41 - protein, 249 aa, Artificial Sequence
  source: /note=“Description of Artificial Sequence Synthetic polypeptide”
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 42 - protein, 14 aa, Artificial Sequence
  source: /note=“Description of Artificial Sequence Synthetic peptide”
GSSTGAVTSGYYPN

SEQ ID NO: 43 - protein, 7 aa, Artificial Sequence
  source: /note=“Description of Artificial Sequence Synthetic peptide”
GTKFLAP

SEQ ID NO: 44 - protein, 9 aa, Artificial Sequence
  source: /note=“Description of Artificial Sequence Synthetic peptide”
ALWYSNRWV

SEQ ID NO: 45 - protein, 5 aa, Artificial Sequence
  source: /note=“Description of Artificial Sequence Synthetic peptide”
RYAMN

SEQ ID NO: 46 - protein, 19 aa, Artificial Sequence
  source: /note=“Description of Artificial Sequence Synthetic peptide”
RIRSKYNNYATYYADSVKG

SEQ ID NO: 47 - protein, 14 aa, Artificial Sequence
  source: /note=“Description of Artificial Sequence Synthetic peptide”
HGNFGNSYLSYFAY

SEQ ID NO: 48 - protein, 125 aa, Artificial Sequence
  source: /note=“Description of Artificial Sequence Synthetic polypeptide”
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 49 - protein, 109 aa, Artificial Sequence; identical to SEQ ID NO: 22
  source: /note=“Description of Artificial Sequence Synthetic polypeptide”

SEQ ID NO: 50 - protein, 249 aa, Artificial Sequence
  source: /note=“Description of Artificial Sequence Synthetic polypeptide”
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 51 - protein, 14 aa, Artificial Sequence
  source: /note=“Description of Artificial Sequence Synthetic peptide”
RSSTGAVTSGYYPN

SEQ ID NO: 52 - protein, 7 aa, Artificial Sequence
  source: /note=“Description of Artificial Sequence Synthetic peptide”
ATDMRPS

SEQ ID NO: 53 - protein, 9 aa, Artificial Sequence
  source: /note=“Description of Artificial Sequence Synthetic peptide”
ALWYSNRWV

SEQ ID NO: 54 - protein, 5 aa, Artificial Sequence
  source: /note=“Description of Artificial Sequence Synthetic peptide”
VYAMN

SEQ ID NO: 55 - protein, 19 aa, Artificial Sequence
  source: /note=“Description of Artificial Sequence Synthetic peptide”
RIRSKYNNYATYYADSVKK

SEQ ID NO: 56 - protein, 14 aa, Artificial Sequence
  source: /note=“Description of Artificial Sequence Synthetic peptide”
HGNFGNSYLSWWAY

SEQ ID NO: 57 - protein, 125 aa, Artificial Sequence
  source: /note=“Description of Artificial Sequence Synthetic polypeptide”
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 58 - protein, 109 aa, Artificial Sequence
  source: /note=“Description of Artificial Sequence Synthetic polypeptide”
QTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 59 - protein, 249 aa, Artificial Sequence
  source: /note=“Description of Artificial Sequence Synthetic polypeptide”
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 60 - protein, 14 aa, Artificial Sequence
  source: /note=“Description of Artificial Sequence Synthetic peptide”
GSSTGAVTSGYYPN

SEQ ID NO: 61 - protein, 7 aa, Artificial Sequence
  source: /note=“Description of Artificial Sequence Synthetic peptide”
GTKFLAP

SEQ ID NO: 62 - protein, 9 aa, Artificial Sequence
  source: /note=“Description of Artificial Sequence Synthetic peptide”
ALWYSNRWV

SEQ ID NO: 63 - protein, 5 aa, Artificial Sequence
  source: /note=“Description of Artificial Sequence Synthetic peptide”
KYAMN

SEQ ID NO: 64 - protein, 19 aa, Artificial Sequence
  source: /note=“Description of Artificial Sequence Synthetic peptide”
RIRSKYNNYATYYADSVKS

SEQ ID NO: 65 - protein, 14 aa, Artificial Sequence
  source: /note=“Description of Artificial Sequence Synthetic peptide”
HGNFGNSYTSYYAY

SEQ ID NO: 66 - protein, 125 aa, Artificial Sequence
  source: /note=“Description of Artificial Sequence Synthetic polypeptide”
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 67 - protein, 109 aa, Artificial Sequence; identical to SEQ ID NO: 22
  source: /note=“Description of Artificial Sequence Synthetic polypeptide”

SEQ ID NO: 68 - protein, 249 aa, Artificial Sequence
  source: /note=“Description of Artificial Sequence Synthetic polypeptide”
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 69 - protein, 14 aa, Artificial Sequence
  source: /note=“Description of Artificial Sequence Synthetic peptide”
RSSTGAVTSGYYPN

SEQ ID NO: 70 - protein, 7 aa, Artificial Sequence
  source: /note=“Description of Artificial Sequence Synthetic peptide”
ATDMRPS

SEQ ID NO: 71 - protein, 9 aa, Artificial Sequence
  source: /note=“Description of Artificial Sequence Synthetic peptide”
ALWYSNRWV

SEQ ID NO: 72 - protein, 5 aa, Artificial Sequence
  source: /note=“Description of Artificial Sequence Synthetic peptide”
GYAMN

SEQ ID NO: 73 - protein, 19 aa, Artificial Sequence
  source: /note=“Description of Artificial Sequence Synthetic peptide”
RIRSKYNNYATYYADSVKE

SEQ ID NO: 74 - protein, 14 aa, Artificial Sequence
  source: /note=“Description of Artificial Sequence Synthetic peptide”
HRNFGNSYLSWFAY

SEQ ID NO: 75 - protein, 125 aa, Artificial Sequence
  source: /note=“Description of Artificial Sequence Synthetic polypeptide”
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 76 - protein, 109 aa, Artificial Sequence; identical to SEQ ID NO: 58
  source: /note=“Description of Artificial Sequence Synthetic polypeptide”

SEQ ID NO: 77 - protein, 249 aa, Artificial Sequence
  source: /note=“Description of Artificial Sequence Synthetic polypeptide”
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 78 - protein, 14 aa, Artificial Sequence
  source: /note=“Description of Artificial Sequence Synthetic peptide”
GSSTGAVTSGYYPN

SEQ ID NO: 79 - protein, 7 aa, Artificial Sequence
  source: /note=“Description of Artificial Sequence Synthetic peptide”
GTKFLAP

SEQ ID NO: 80 - protein, 9 aa, Artificial Sequence
  source: /note=“Description of Artificial Sequence Synthetic peptide”
ALWYSNRWV

SEQ ID NO: 81 - protein, 5 aa, Artificial Sequence
  source: /note=“Description of Artificial Sequence Synthetic peptide”
VYAMN

SEQ ID NO: 82 - protein, 19 aa, Artificial Sequence
  source: /note=“Description of Artificial Sequence Synthetic peptide”
RIRSKYNNYATYYADSVKK

SEQ ID NO: 83 - protein, 14 aa, Artificial Sequence
  source: /note=“Description of Artificial Sequence Synthetic peptide”
HGNFGNSYISWWAY

SEQ ID NO: 84 - protein, 125 aa, Artificial Sequence
  source: /note=“Description of Artificial Sequence Synthetic polypeptide”
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 85 - protein, 109 aa, Artificial Sequence; identical to SEQ ID NO: 22
  source: /note=“Description of Artificial Sequence Synthetic polypeptide”

SEQ ID NO: 86 - protein, 249 aa, Artificial Sequence
  source: /note=“Description of Artificial Sequence Synthetic polypeptide”
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 87 - protein, 14 aa, Artificial Sequence
  source: /note=“Description of Artificial Sequence Synthetic peptide”
GSSTGAVTSGNYPN

SEQ ID NO: 88 - protein, 7 aa, Artificial Sequence
  source: /note=“Description of Artificial Sequence Synthetic peptide”
GTKFLAP

SEQ ID NO: 89 - protein, 9 aa, Artificial Sequence
  source: /note=“Description of Artificial Sequence Synthetic peptide”
VLWYSNRWV

SEQ ID NO: 90 - protein, 5 aa, Artificial Sequence
  source: /note=“Description of Artificial Sequence Synthetic peptide”
SYAMN

SEQ ID NO: 91 - protein, 19 aa, Artificial Sequence
  source: /note=“Description of Artificial Sequence Synthetic peptide”
RIRSKYNNYATYYADSVKG

SEQ ID NO: 92 - protein, 14 aa, Artificial Sequence
  source: /note=“Description of Artificial Sequence Synthetic peptide”
HGNFGNSYVSWWAY

SEQ ID NO: 93 - protein, 125 aa, Artificial Sequence
  source: /note=“Description of Artificial Sequence Synthetic polypeptide”
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 94 - protein, 109 aa, Artificial Sequence
  source: /note=“Description of Artificial Sequence Synthetic polypeptide”
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 95 - protein, 249 aa, Artificial Sequence
  source: /note=“Description of Artificial Sequence Synthetic polypeptide”
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 96 - protein, 14 aa, Artificial Sequence
  source: /note=“Description of Artificial Sequence Synthetic peptide”
GSSTGAVTSGNYPN

SEQ ID NO: 97 - protein, 7 aa, Artificial Sequence
  source: /note=“Description of Artificial Sequence Synthetic peptide”
GTKFLAP

SEQ ID NO: 98 - protein, 9 aa, Artificial Sequence
  source: /note=“Description of Artificial Sequence Synthetic peptide”
VLWYSNRWV

SEQ ID NO: 99 - protein, 5 aa, Artificial Sequence
  source: /note=“Description of Artificial Sequence Synthetic peptide”
KYAMN

SEQ ID NO: 100 - protein, 19 aa, Artificial Sequence
  source: /note=“Description of Artificial Sequence Synthetic peptide”
RIRSKYNNYATYYADSVKD

SEQ ID NO: 101 - protein, 14 aa, Artificial Sequence
  source: /note=“Description of Artificial Sequence Synthetic peptide”
HGNFGNSYISYWAY

SEQ ID NO: 102 - protein, 125 aa, Artificial Sequence; identical to SEQ ID NO: 30
  source: /note=“Description of Artificial Sequence Synthetic polypeptide”

SEQ ID NO: 103 - protein, 109 aa, Artificial Sequence; identical to SEQ ID NO: 94
  source: /note=“Description of Artificial Sequence Synthetic polypeptide”

SEQ ID NO: 104 - protein, 249 aa, Artificial Sequence
  source: /note=“Description of Artificial Sequence Synthetic polypeptide”
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 105 - protein, 14 aa, Artificial Sequence
  source: /note=“Description of Artificial Sequence Synthetic peptide”
GSSTGAVTSGNYPN

SEQ ID NO: 106 - protein, 7 aa, Artificial Sequence
  source: /note=“Description of Artificial Sequence Synthetic peptide”
GTKFLAP

SEQ ID NO: 107 - protein, 9 aa, Artificial Sequence
  source: /note=“Description of Artificial Sequence Synthetic peptide”
VLWYSNRWV

SEQ ID NO: 108 - protein, 5 aa, Artificial Sequence
  source: /note=“Description of Artificial Sequence Synthetic peptide”
SYAMN

SEQ ID NO: 109 - protein, 19 aa, Artificial Sequence
  source: /note=“Description of Artificial Sequence Synthetic peptide”
RIRSKYNNYATYYADSVKG

SEQ ID NO: 110 - protein, 14 aa, Artificial Sequence
  source: /note=“Description of Artificial Sequence Synthetic peptide”
HGNFGNSYVSWWAY

SEQ ID NO: 111 - protein, 125 aa, Artificial Sequence
  source: /note=“Description of Artificial Sequence Synthetic polypeptide”
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSS

SEQ ID NO: 112 - protein, 109 aa, Artificial Sequence; identical to SEQ ID NO: 94
  source: /note=“Description of Artificial Sequence Synthetic polypeptide”

SEQ ID NO: 113 - protein, 249 aa, Artificial Sequence
  source: /note=“Description of Artificial Sequence Synthetic polypeptide”
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 114 - protein, 4 aa, Artificial Sequence
  source: /note=“Description of Artificial Sequence Synthetic peptide”
GGGG

SEQ ID NO: 115 - protein, 5 aa, Artificial Sequence
  source: /note=“Description of Artificial Sequence Synthetic peptide”
GGGGS

SEQ ID NO: 116 - protein, 5 aa, Artificial Sequence
  source: /note=“Description of Artificial Sequence Synthetic peptide”
GGGGQ

SEQ ID NO: 117 - protein, 6 aa, Artificial Sequence
  source: /note=“Description of Artificial Sequence Synthetic peptide”
PGGGGS

SEQ ID NO: 118 - protein, 6 aa, Artificial Sequence
  source: /note=“Description of Artificial Sequence Synthetic peptide”
PGGDGS

SEQ ID NO: 119 - protein, 6 aa, Artificial Sequence
  source: /note=“Description of Artificial Sequence Synthetic peptide”
SGGGGS

SEQ ID NO: 120 - protein, 9 aa, Artificial Sequence
  source: /note=“Description of Artificial Sequence Synthetic peptide”
GGGGSGGGS

SEQ ID NO: 121 - protein, 10 aa, Artificial Sequence
  source: /note=“Description of Artificial Sequence Synthetic peptide”
GGGGSGGGGS

SEQ ID NO: 122 - protein, 15 aa, Artificial Sequence
  source: /note=“Description of Artificial Sequence Synthetic peptide”
GGGGSGGGGSGGGGS

SEQ ID NO: 123 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 124 - protein, 96 aa, Callithrix jacchus
QDGNEEMGDTTQNPYKVSISGTTVTLTCPRYDGHEIKWLVNSQNKEGHEDHLLLEDFSEMEQSGYYACLSKETPAEEASHYLYLKARVCENCVEVD

SEQ ID NO: 125 - protein, 96 aa, Saguinus oedipus; identical to SEQ ID NO: 124

SEQ ID NO: 126 - protein, 96 aa, Saimiri sciureus
QDGNEEIGDTTQNPYKVSISGTTVTLTCPRYDGQEIKWLVNDQNKEGHEDHLLLEDFSEMEQSGYYACLSKETPTEEASHYLYLKARVCENCVEVD

SEQ ID NO: 127 - protein, 5 aa, Artificial Sequence
  source: /note=“Description of Artificial Sequence Synthetic peptide”
SYGMH

SEQ ID NO: 128 - protein, 17 aa, Artificial Sequence
  source: /note=“Description of Artificial Sequence Synthetic peptide”
VIWYDGSNKYYVDSVKG

SEQ ID NO: 129 - protein, 11 aa, Artificial Sequence
  source: /note=“Description of Artificial Sequence Synthetic peptide”
DGWQQLAPFDY

SEQ ID NO: 130 - protein, 16 aa, Artificial Sequence
  source: /note=“Description of Artificial Sequence Synthetic peptide”
RSSQSLVHSDGNTYLS

SEQ ID NO: 131 - protein, 7 aa, Artificial Sequence
  source: /note=“Description of Artificial Sequence Synthetic peptide”
KISNRFS

SEQ ID NO: 132 - protein, 9 aa, Artificial Sequence
  source: /note=“Description of Artificial Sequence Synthetic peptide”
MQATQLPRT

SEQ ID NO: 133 - protein, 120 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSA

SEQ ID NO: 134 - protein, 112 aa, Artificial Sequence
  source: /note=“Description of Artificial Sequence Synthetic polypeptide”
DIVMTQTPLSSPVTLGQPASISCRSSQSLVHSDGNTYLSWLHQRPGQPPRLLIYKISNRFSGVPDRFSGSGAGTAFTLKISRVEAEDVGVYYCMQATQLPRTFGQGTKVEIK

SEQ ID NO: 135 - protein, 324 aa, Artificial Sequence
  source: /note=“Description of Artificial Sequence Synthetic polypeptide”
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 136 - protein, 107 aa, Artificial Sequence
  source: /note=“Description of Artificial Sequence Synthetic polypeptide”
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 137 - protein, 444 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 138 - protein, 219 aa, Artificial Sequence
  source: /note=“Description of Artificial Sequence Synthetic polypeptide”
DIVMTQTPLSSPVTLGQPASISCRSSQSLVHSDGNTYLSWLHQRPGQPPRLLIYKISNRFSGVPDRFSGSGAGTAFTLKISRVEAEDVGVYYCMQATQLPRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 139 - protein, 124 aa, Artificial Sequence
  source: /note=“Description of Artificial Sequence Synthetic polypeptide”
QVQLVESGGGVVQSGRSLRLSCAASGFTFRNYGMHWVRQAPGKGLEWVAVIWYDGSDKYYADSVRGRFTISRDNSKNTLYLQMNSLRAEDTAVYYCARDGYDILTGNPRDFDYWGQGTLVTVSA

SEQ ID NO: 140 - protein, 112 aa, Artificial Sequence
  source: /note=“Description of Artificial Sequence Synthetic polypeptide”
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGQGTKVEIK

SEQ ID NO: 141 - protein, 448 aa, Artificial Sequence
  source: /note=“Description of Artificial Sequence Synthetic polypeptide”
QVQLVESGGGVVQSGRSLRLSCAASGFTFRNYGMHWVRQAPGKGLEWVAVIWYDGSDKYYADSVRGRFTISRDNSKNTLYLQMNSLRAEDTAVYYCARDGYDILTGNPRDFDYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 142 - protein, 219 aa, Artificial Sequence
  source: /note=“Description of Artificial Sequence Synthetic polypeptide”
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 143 - protein, 20 aa, Artificial Sequence
  source: /note=“Description of Artificial Sequence Synthetic peptide”
  SITE (1)..(20): /note=“This sequence may encompass 1-4 ‘Gly Gly Gly Gly Ser’ repeating units”
GGGGSGGGGSGGGGSGGGGS

SEQ ID NO: 144 - protein, 5 aa, Artificial Sequence
  source: /note=“Description of Artificial Sequence Synthetic peptide”
GGGGS